(19) United States
(12) Patent Application Publication
(10) Pub. No.: US 2024/0382474 A1
(43) Pub. Date: Nov. 21, 2024

(54) ENPP1 MODULATORS AND USES THEREOF

(71) Applicant: Vir Biotechnology, Inc., San Francisco, CA (US)

(72) Inventors: Roland D. SAITO, San Mateo, CA (US); Winston C. TSE, Redwood City, CA (US)

(21) Appl. No.: 18/662,794

(22) Filed: May 13, 2024

Related U.S. Application Data
(63) Continuation of application No. PCT/US2022/049844, filed on Nov. 14, 2022.
(60) Provisional application No. 63/279,526, filed on Nov. 15, 2021.

(51) Int. Cl. A61K 31/4709; A61K 31/47; A61K 31/517; A61P 35/00; C07D 215/233; C07D 215/46; C07D 239/94; C07D 401/12

(52) U.S. Cl. CPC A61K 31/4709; A61K 31/47; A61K 31/517; A61P 35/00; C07D 215/233; C07D 215/46; C07D 239/94; C07D 401/12

(57) ABSTRACT

The disclosure provides enantiomeric quinoline and aza-quinazolines based on the ENPP1 modulators of Formula (I) or Formula (II) and salts thereof, and their use for the modulation of ENPP1 activity.

### CROSS-REFERENCE

This application is a continuation of International Patent Application No. PCT/US2022/049844, filed on Nov. 14, 2022, which claims the benefit of U.S. Provisional Application No. 63/279,526 filed on Nov. 15, 2021, the entire contents of which are herein incorporated by reference.

### BACKGROUND OF THE INVENTION

Ectonucleotide pyrophosphate/phosphodiesterase 1 (ENPP1) catalyzes the breakdown of extracellular adenosine triphosphate (ATP) into adenosine monophosphate (AMP) and pyrophosphate (PP)—an important inhibitor of tissue calcification. Additionally, ENPP1 degrades cyclic guanosine monophosphate-adenosine monophosphate (cGAMP), a secondary messenger molecule that mediates the upregulation of type I interferons and other inflammatory cytokines and chemokines by activating stimulator of interferon genes (STING). Therapeutically, the use of ENPP1 modulators may have particular advantage in applications such as anti-viral therapy, anti-bacterial therapy, immuno-therapy, immunological adjuvants, pyrophosphate inhibitors, anti-cancer therapy, and anti-inflammatory therapy. Therefore, there exists a need for the development of ENPP1 modulators for the treatment of disease.

### SUMMARY OF THE INVENTION

In certain aspects the present disclosure provides a compound represented by the structure of Formula (I):

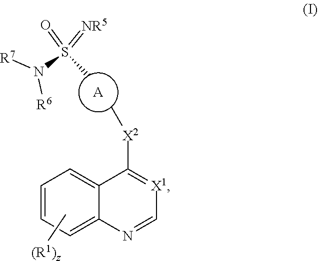

or a pharmaceutically acceptable salt thereof, wherein:
X¹ is selected from N and C(R²);
X² is selected from O, S, C(R³)₂, and N(R⁴);
R¹ is selected from:
   halogen, —OR¹¹, —SR¹¹, —N(R¹¹)₂, —C(O)R¹¹, —C(O)N(R¹¹)₂, —
N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, —CN; and
   C₁₋₆ alkyl, C₂₋₆ alkenyl, C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle;
R² is selected from:
   hydrogen, halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, —CN; and
   C₁₋₆ alkyl, C₂₋₆ alkenyl, C₂₋₆ alkynyl, each of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle;
each R³ is independently selected from:
   hydrogen, halogen, —OR¹³, —SR¹³, —N(R¹³)₂, —NO₂, and —CN; and
   C₁₋₆ alkyl optionally substituted with one or more substituents independently selected from halogen, —OR¹³, —SR¹³, —N(R¹³)₂, —NO₂, and —CN;
R⁴ is selected from hydrogen, C₁₋₆ alkyl, and C₃₋₆ carbocycle wherein C₁₋₆ alkyl and C₃₋₆ carbocycle are each optionally substituted with one or more substituents independently selected from:
   halogen, —OR¹⁴, —SR¹⁴, —N(R¹⁴)₂, —NO₂, and —CN;
Ring A is selected from an optionally substituted C₃₋₁₀ carbocycle and optionally substituted 3- to 10-membered heterocycle wherein substituents on Ring A are independently selected at each occurrence from:
   halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
      C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN;
R⁵, R⁶, and R⁷ are each independently selected from hydrogen, optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from:
   halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN;
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle; and
   C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle;
each R¹¹, R¹², R¹³, R¹⁴, R¹⁵, and R¹⁶ is independently selected from:
   hydrogen; and
   C₁₋₆ alkyl, C₂₋₆ alkenyl, C₂₋₆ alkynyl, C₃₋₈ carbocycle, and 3- to 8-membered heterocycle, each of which is optionally substituted with one or more substituents independently selected from halogen, —OH, —CN, —NO₂, —NH₂, ═O, ═S, —C₁₋₆ haloalkyl, and —O—C₁₋₆ alkyl; and
z is selected from 0-3.

In certain aspects, the present disclosure provides a compound represented by the structure of Formula (II):

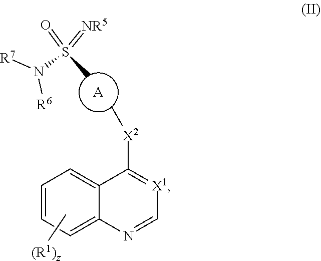

X¹ is selected from N and C(R²);
X² is selected from O, S, C(R³)₂, and N(R⁴);
R¹ is selected from:
   halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, —CN; and
   C₁₋₆ alkyl, C₂₋₆ alkenyl, C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle;
R² is selected from:
   hydrogen, halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, —CN; and
   C₁₋₆ alkyl, C₂₋₆ alkenyl, C₂₋₆ alkynyl, each of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle;
each R³ is independently selected from:
   hydrogen, halogen, —OR¹³, —SR¹³, —N(R¹³)₂, —NO₂, and —CN; and
   C₁₋₆ alkyl optionally substituted with one or more substituents independently selected from halogen, —OR¹³, —SR¹³, —N(R¹³)₂, —NO₂, and —CN;
R⁴ is selected from hydrogen, C₁₋₆ alkyl, and C₃₋₆ carbocycle, wherein C₁₋₆ alkyl and C₃₋₆ carbocycle are each optionally substituted with one or more substituents independently selected from: halogen, —OR¹⁴, —SR¹⁴, —N(R¹⁴)₂, —NO₂, and —CN;
Ring A is selected from an optionally substituted C₃₋₁₀ carbocycle and optionally substituted 3- to 10-membered heterocycle wherein substituents on Ring A are independently selected at each occurrence from:
   halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
      C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN;
R⁵, R⁶, and R⁷ are each independently selected from hydrogen, optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from:
   halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN;
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle; and
   C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O) OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle;
each R¹¹, R¹², R¹³, R¹⁴, R¹⁵, and R¹⁶ is independently selected from:
   hydrogen; and
   C₁₋₆ alkyl, C₂₋₆ alkenyl, C₂₋₆ alkynyl, C₃₋₈ carbocycle, and 3- to 8-membered heterocycle, each of which is optionally substituted with one or more substituents independently selected from halogen, —OH, —CN, —NO₂, —NH₂, ═O, ═S, —C₁₋₆ haloalkyl, and —O—C₁₋₆ alkyl; and
z is selected from 0-3.

In certain embodiments, the present disclosure provides a pharmaceutical composition comprising a compound or salt of Formula (I) or (II), and a pharmaceutically acceptable excipient.

In certain embodiments, the present disclosure provides a method of inhibiting ENPP1 in a subject in need thereof, comprising administering to the subject a compound or salt of Formula (I) or (II).

In another aspect, the present disclosure provides a method treating cancer in a subject in need thereof, comprising administrating to the subject a compound of Formula (I) or (II), or a pharmaceutically acceptable salt thereof, or a pharmaceutical composition comprising a compound or salt of Formula (I) or (II) and a pharmaceutically acceptable excipient.

In another aspect, the present disclosure provides a compound of Formula (I) or (II), or a pharmaceutically acceptable salt thereof, use in treating cancer in a subject in need thereof.

In another aspect, the present disclosure provides the use of a compound of Formula (I) or (II), or a pharmaceutically acceptable salt thereof, for the manufacture of a medicament for the treatment of cancer.

In some embodiments, the cancer comprises a solid tumor. In some embodiments, the cancer is selected from: a sarcoma, a carcinoma, and a lymphoma. In some embodiments, the cancer is selected from breast cancer, colon cancer, bladder cancer, prostate cancer, ovarian cancer, glioblastoma, and lung cancer.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides a graph of tumor volume in mice bearing MC38 tumor xenografts, including mice dosed with Compound 26.

FIG. 2A provides a graph of tumor volume in mice bearing MC38 tumor xenografts, including mice dosed with Compounds 26 and 56.

FIG. 2B provides a graph of tumor volume in mice bearing MC38 tumor xenografts, including mice dosed with Compounds 26 and 56.

FIG. 3A provides a Kaplan-Meier survival curve for mice bearing MC38 tumor xenografts, including mice dosed with Compounds 26 and 56.

FIG. 3B provides a graph of mean body weights for mice bearing MC38 tumor xenografts, including mice dosed with Compounds 26 and 56.

FIG. 4A provides a graph of tumor volume at Day 10 in mice bearing MC38 tumor xenografts, including mice dosed with Compounds 26 and 56.

FIG. 4B provides a graph of interferon beta (IFNbeta) levels (pg/g) in MC38 tumors at Day 10 in mice bearing MC38 tumor xenografts, including mice dosed with Compounds 26 and 56.

FIG. 4C provides a graph of interferon γ-induced protein 10 kDa (IP-10) levels (pg/g) in MC38 tumors at Day 10 in mice bearing MC38 tumor xenografts, including mice dosed with Compounds 26 and 56.

FIG. 4D provides a graph of macrophage inflammatory protein-1-beta (MIP-1beta) levels (pg/g) in MC38 tumors at Day 10 in mice bearing MC38 tumor xenografts, including mice dosed with Compounds 26 and 56.

FIG. 4E provides a graph of monocyte-chemotactic protein 3 (MCP-3) levels (pg/g) in MC38 tumors at Day 10 in mice bearing MC38 tumor xenografts, including mice dosed with Compounds 26 and 56.

FIG. 4F provides a graph of eotaxin levels (pg/g) in MC38 tumors at Day 10 in mice bearing MC38 tumor xenografts, including mice dosed with Compounds 26 and 56.

FIG. 5A provides a graph of tumor volume in mice bearing MC38 tumor xenografts, including mice dosed with Compounds 26 and 56.

FIG. 5B provides a Kaplan-Meier survival curve for mice bearing MC38 tumor xenografts, including mice dosed with Compounds 26 and 56.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”. “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e., C₁-C₁₂ alkyl). The alkyl is attached to the remainder of the molecule through a single bond. In certain embodiments, an alkyl comprises one to twelve carbon atoms (i.e., C₁-C₁₂ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈alkyl). In other embodiments, an alkyl comprises two to five carbon atoms (i.e., C₂-C₅ alkyl). In other embodiments, an alkyl comprises three to five carbon atoms (i.e., C₃-C₅ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₅ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein. In certain embodiments, an alkylene comprises one to ten carbon atoms (i.e., C₁-C₁₀ alkylene). In certain embodiments, an alkylene comprises one to eight carbon atoms (i.e., C₁-C₅ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term-C_{x-y} alkylene-refers to a substituted or unsubstituted alkylene chain with from x to y carbons in the alkylene chain. For example, —C₁₋₆ alkylene- may be selected from methylene, ethylene, propylene, butylene, pentylene, and hexylene, any one of which is optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term-C_{x-y} alkenylene-refers to a substituted or unsubstituted alkenylene chain with from x to y carbons in the alkenylene chain. For example, —C₂₋₆ alkenylene may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkynylene chain. For example, —C₂₋₆ alkynylene- may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo| 2.2.1|heptanyl), norbornenyl, decalinyl, 7,7-dimethyl-bicyclo| 2.2.1|heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2)π-electron system in accordance with the Hückel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms—any of which is optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridavinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

“Heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperavinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Heterocycloalkyl may be optionally substituted by one or more substituents such as those substituents described herein.

The term “heteroaryl” refers to a radical derived from a 3- to 12-membered aromatic ring radical that comprises one to eleven carbon atoms and at least one heteroatom wherein each heteroatom may be selected from N, O, and S. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl may be optionally substituted by one or more substituents such as those substituents described herein.

An “X-membered heterocycle” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula-O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radicals, each halogen may be independently selected for example, 1-chloro,2-fluoroethane.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂. —R^{b}—N(R^{a})C(O)OR^{a}, —R^{b}—N(R^{a})C(O)R^{a}, —R^{b}—N(R^{a})S(O)ₜR^{a} (where tis 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each Re is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject.” “individual.” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described herein, that suffers from, or is at risk for, a pathology to be prophylactically or therapeutically treated with a compound or salt described herein.

The terms “administer”. “administered”. “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”. “administered”. “administers” and “administering” a compound should be understood to mean providing a compound of the invention or a salt of a compound of the invention to the individual in need.

As used herein. “treatment” or “treating” refers to an approach for obtaining beneficial or desired results with respect to a disease, disorder, or medical condition including, but not limited to. a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “inhibit”, “selective inhibition” or “selectively inhibit” refers to an agent's (chemical or biological) ability to preferentially reduce the target signaling activity as compared to off-target signaling activity, via direct or indirect interaction with the target.

A “therapeutic effect,” as used herein, encompasses a therapeutic benefit and/or a prophylactic benefit as described above. A prophylactic effect includes delaying or eliminating the appearance of a disease or condition, onset of symptoms of a disease or condition, slowing, halting, or reversing the progression of a disease or condition, or any combination thereof.

### Compounds

In some aspects, the present disclosure provides a compound represented by the structure of Formula (I):

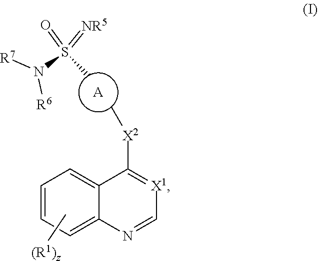

or a pharmaceutically acceptable salt thereof, wherein:
X¹ is selected from N and C(R²);
X² is selected from O, S, C(R³)₂, and N(R⁴);
R¹ is selected from:
   halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹), —S(O)₂R¹¹, —S(O)(NR¹¹)R¹¹, —S(NR¹¹)₂R¹¹, —NO₂, —CN; and
   C₁₋₆ alkyl, C₂₋₆ alkenyl, C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle;
R² is selected from:
   hydrogen, halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, —CN; and
   C₁₋₆ alkyl, C₂₋₆ alkenyl, C₂₋₆ alkynyl, each of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle;
each R³ is independently selected from:
   hydrogen, halogen, —OR¹³, —SR¹³, —N(R¹³)₂, —NO₂, and —CN; and
   C₁₋₆ alkyl optionally substituted with one or more substituents independently selected from halogen, —OR¹³, —SR¹³, —N(R¹³)₂, —NO₂, and —CN;
R⁴ is selected from hydrogen, C₁₋₆ alkyl, and C₃₋₆ carbocycle wherein C₁₋₆ alkyl and C₃₋₆ carbocycle are each optionally substituted with one or more substituents independently selected from:
   halogen, —OR¹⁴, —SR¹⁴, —N(R¹⁴)₂, —NO₂, and —CN;
Ring A is selected from an optionally substituted C₃₋₁₀ carbocycle and optionally substituted 3- to 10-membered heterocycle wherein substituents on Ring A are independently selected at each occurrence from:
   halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
      C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN;
R⁵, R⁶, and R⁷ are each independently selected from hydrogen, optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from:
   halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN;
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle; and
   C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle;
each R¹¹, R¹², R¹³, R¹⁴, R¹⁵, and R¹⁶ is independently selected from:
   hydrogen; and
   C₁₋₆ alkyl, C₂₋₆ alkenyl, C₂₋₆ alkynyl, C₃₋₈ carbocycle, and 3- to 8-membered heterocycle, each of which is optionally substituted with one or more substituents independently selected from halogen, —OH, —CN, —NO₂, —NH₂, ═O, ═S, —C₁₋₆ haloalkyl, and —O—C₁₋₆ alkyl; and
z is selected from 0-3

In some embodiments, for a compound or salt of Formula (I), z is selected from 0-2. In some embodiments, z is selected from 0 and 1. In some embodiments, z is selected from 1-3. In some embodiments, z is selected from 2 and 3. In some embodiments, z is selected from 1 and 2. In some embodiments, z is 0). In some embodiments, z is 1. In some embodiments, z is 2. In some embodiments, z is 3.

In some embodiments, for a compound or salt of Formula (I). X¹ is selected from N and C(R²), wherein R² is selected from hydrogen, halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²) S(O)₂R¹², —S(O)(NR¹²)R¹², —S(NR¹²)₂R¹², —NO₂, and —CN. In some embodiments, X¹ is selected from N and C(R²), wherein R² is selected from hydrogen, halogen, —OR¹², —N(R¹²)₂, —NO₂, and —CN. In some embodiments, X¹ is selected from N and C(R²), wherein R² is selected from hydrogen and —CN. In some embodiments, X¹ is selected from N, C(H), and C(CN).

In some embodiments, for a compound or salt of Formula (I). X¹ is selected from N and C(R²), wherein R² is selected from C₁₋₆ alkyl, C₂₋₆ alkenyl, C₂₋₆ alkynyl, each of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle. In some embodiments, X¹ is selected from N and C(R²), wherein R² is selected from C₁₋₆ alkyl optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle. In some embodiments, X¹ is selected from N and C(R²), wherein R² is selected from C₁₋₆ alkyl optionally substituted with one or more substituents independently selected from halogen, —OR¹², —N(R¹²)₂, —C(O)R¹², —NO₂, ═O, —CN, C₃₋₆ carbocycle, and 3- to 6-membered heterocycle. In some embodiments, X¹ is selected from N and C(R²), wherein R² is selected from C₂₋₆ alkenyl and C₂₋₆ alkynyl, each of which is optionally substituted with one or more substituents independently selected from halogen, —OR¹², —SR¹², —N(R¹²)₂, —C(O)R¹², —C(O)N(R¹²)₂. —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle.

In some embodiments, for a compound or salt of Formula (I), X² is selected from O and S. In some embodiments, X² is O. In some embodiments, X² is selected from C(R³), wherein R³ is selected from hydrogen, halogen, —OR¹³, —SR¹³, —N(R¹³)₂, —NO₂, and —CN. In some embodiments, X² is selected from C(R³), wherein R³ is C₁₋₆ alkyl optionally substituted with one or more substituents independently selected from halogen, —OR¹³, —SR¹³, —N(R¹³)₂, —NO₂, and —CN. In some embodiments. X² is selected from N(R⁴), wherein R⁴ is hydrogen. In some embodiments, X² is selected from N(R⁴), wherein R⁴ is selected from C₁₋₆ alkyl and C₃₋₆ carbocycle each of which is optionally substituted with one or more substituents independently selected from: halogen, —OR¹⁴, —SR¹⁴, —N(R¹⁴)₂, —NO₂, and —CN. In some embodiments, X² is N(H). In some embodiments, X² is selected from O and N(H).

In some embodiments, for a compound or salt of Formula (I). R¹ is selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN. In some embodiments. R¹ is selected from halogen, —OR¹¹, —N(R¹¹)₂, —C(O)R¹¹, —C(O)N(R¹¹)₂, —NO₂, and —CN.

In some embodiments, for a compound or salt of Formula (I), R¹ is selected from C₁₋₆ alkyl. C₂₋₆ alkenyl, C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle. In some embodiments, R¹ is selected from C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle. In some embodiments, R¹ is selected from C₂₋₆ alkenyl and C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle.

In some embodiments, for a compound or salt of Formula (I). R¹ is selected from: halogen, —OR¹¹, —N(R¹¹)₂, —C(O)R¹¹, —NO₂, —CN, and C₁₋₆ alkyl optionally substituted with one or more substituents independently selected from halogen, —OR¹¹, —N(R¹¹)₂, —C(O)R¹¹, —NO₂, and —CN. In some embodiments, R¹ is selected from —OR¹¹ and R¹¹ is selected from C₁₋₆ alkyl and C₁₋₆ haloalkyl. In some embodiments, R¹ is selected from —OR¹¹ and R¹¹ is selected from C₁₋₆ alkyl. In some embodiments. R¹ is selected from —OR¹¹ and R¹¹ is selected from C₁₋₆ haloalkyl. In some embodiments. R¹ is selected from methoxy and —OC(H) F₂. In some embodiments, R¹ is methoxy. In some embodiments. R¹ is —OC(H)F₂.

In some embodiments, for a compound or salt of Formula (I), Ring A is C₃₋₁₀ carbocycle optionally substituted with one or more substituents independently selected from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (I), Ring A is C₃₋₁₀ carbocycle optionally substituted with one or more substituents independently selected from: halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl. In some embodiments, Ring A is C₃₋₁₀ carbocycle optionally substituted with one or more substituents independently selected from C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (I), Ring A is selected from optionally substituted C₃₋₉ carbocycle, optionally substituted C₃₋₈ carbocycle, optionally substituted C₃₋₇ carbocycle, optionally substituted Ca-6 carbocycle, optionally substituted C₃₋₅ carbocycle, and optionally substituted C₃₋₄ carbocycle. In some embodiments, Ring A is selected from optionally substituted C₄₋₁₀ carbocycle, optionally substituted C₅₋₁₀ carbocycle, optionally substituted C₆₋₁₀ carbocycle, optionally substituted C₇₋₁₀ carbocycle, optionally substituted C₈₋₁₀ carbocycle, and optionally substituted C₉₋₁₀ carbocycle. In some embodiments, Ring A is selected from optionally substituted C₃ carbocycle, optionally substituted C₄ carbocycle, optionally substituted C₅ carbocycle, optionally substituted C₆ carbocycle, optionally substituted C₇ carbocycle, optionally substituted C₈ carbocycle, optionally substituted C₉ carbocycle, and optionally substituted C₁₀ carbocycle. In some embodiments, Ring A is selected from an optionally substituted saturated C₃₋₁₀ carbocycle and an optionally substituted unsaturated C₃₋₁₀ carbocycle.

In some embodiments, for a compound or salt of Formula (I), Ring A is C₃₋₆ carbocycle optionally substituted with one or more substituents independently selected from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (I), Ring A is C₃₋₆ carbocycle optionally substituted with one or more substituents independently selected from: halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl. In some embodiments, Ring A is C₃₋₁₀ carbocycle optionally substituted with one or more substituents independently selected from C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (I), Ring A is selected from C₃₋₆ carbocycle optionally substituted with one or more substituents independently selected from: halogen, —OR¹⁵, —N(R¹⁵)₂, —C(O)R¹⁵, —NO₂, ═O, —CN, and C₁₋₆ haloalkyl. In some embodiments, Ring A is selected from cyclopropyl, cyclobutyl, cyclopentyl, cyclohexyl, and phenyl, each of which is optionally substituted with one or more substituents independently selected from: halogen, —OR¹⁵, —N(R¹⁵)₂, —C(O)R¹⁵, —NO₂, ═O, —CN, and C₁₋₆ haloalkyl. In some embodiments, Ring A is phenyl optionally substituted with one or more substituents independently selected from: halogen, —OR¹⁵, —N(R¹⁵)₂, —C(O)R¹⁵, —NO₂, ═O, —CN, and C₁₋₆ haloalkyl. In some embodiments, Ring A is phenyl optionally substituted with one or more substituents independently selected from: fluorine, chlorine, CN, and C₁₋₆ haloalkyl. In some embodiments, Ring A is phenyl optionally substituted with fluorine.

In some embodiments, for a compound or salt of Formula (I), Ring A is 3- to 10-membered heterocycle optionally substituted with one or more substituents independently selected from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (I), Ring A is 3- to 10-membered heterocycle optionally substituted with one or more substituents independently selected from: halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵. —S(O)R¹⁵, —S(O)₂R¹⁵, —NO₂, ═O, —CN, and C₁₋₆ haloalkyl. In some embodiments, Ring A is 3- to 10-membered heterocycle optionally substituted with one or more substituents independently selected from: C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂. —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (I), Ring A is an optionally substituted 3- to 10-membered heterocycle comprising at least one heteroatom selected from nitrogen, oxygen, and sulfur. In some embodiments, Ring A is selected from an optionally substituted 3- to 9-membered heterocycle, an optionally substituted 3- to 8-membered heterocycle, an optionally substituted 3- to 7-membered heterocycle, an optionally substituted 3- to 6-membered heterocycle, an optionally substituted 3- to 5-membered heterocycle, and an optionally substituted 3- to 4-membered heterocycle. In some embodiments, Ring A is selected from an optionally substituted 4- to 10-membered heterocycle, an optionally substituted 5- to 10-membered heterocycle, an optionally substituted 6- to 10-membered heterocycle, an optionally substituted 7- to 10-membered heterocycle, an optionally substituted 8- to 10-membered heterocycle, and an optionally substituted 9- to 10-membered heterocycle. In some embodiments, Ring A selected from is an optionally substituted 4- to 10-membered saturated heterocycle and optionally substituted 4- to 10-membered unsaturated heterocycle.

In some embodiments, for a compound or salt of Formula (I), wherein two of R⁵, R⁶, and R⁷ are selected from hydrogen and the other of R⁵, R⁶, and R⁷ is selected from C₁₋₆ alkyl optionally substituted with one or more substituents independently selected from:
—OR¹⁶, —N(R¹⁶)₂, —C(O)R¹⁶, —NO₂, ═O, —CN; and
C₃₋₆ carbocycle and 5- to 6-membered heterocycle optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

In some embodiments, for a compound or salt of Formula (I), at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from:
halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN;
C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶—NO₂, ═O, ═S, ═N(R¹⁶), —CN, C₃₋₁₂ carbocycle, and 3- to 12-membered heterocycle; and
C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O) OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle.

In some embodiments, for a compound or salt of Formula (I), at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), and —CN. In some embodiments, at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: halogen, —OR¹⁶, —N(R¹⁶)₂, —C(O)R¹⁶, —C(O)N(R¹⁶)₂, —NO₂, ═O, ═S, ═N(R¹⁶), and —CN.

In some embodiments, for a compound or salt of Formula (I), at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle. In some embodiments, at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₁₋₆ alkyl, C₂₋₆ alkenyl, C₂₋₆ alkynyl, each of which is optionally substituted with one or more substituents independently selected from halogen, —OR¹⁶, —N(R¹⁶)₂, —C(O)R¹⁶, —C(O)N(R¹⁶)₂, —NO₂, ═O, ═S, ═N(R¹⁶), —CN, C₃₋₁₂ carbocycle, and 3- to 12-membered heterocycle.

In some embodiments, for a compound or salt of Formula (I), at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle. In some embodiments, at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₃₋₁₂ carbocycle and 3- to 12-membered heterocycle each of which is optionally substituted with one or more substituents independently selected from halogen, —OR¹⁶, —N(R¹⁶)₂, —C(O)R¹⁶, —C(O)N(R¹⁶)₂, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle.

In some embodiments, for a compound or salt of Formula (I), two of R⁵, R⁶, and R⁷ are hydrogen, and the other of R⁵, R⁶, and R⁷ is selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from:
halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN;
C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle; and
C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle.

In some embodiments, for a compound or salt of Formula (I), two of R⁵, R⁶, and R⁷ are hydrogen, and the other of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂ R¹⁶, —S(O)(NR¹⁶)R¹⁶, —S(NR¹⁶)₂R¹⁶, —NO₂, ═O, ═S, ═N(R¹⁶), and —CN. In some embodiments, two of R⁵, R⁶, and R⁷ are hydrogen, and the other R⁵, R⁶, and R⁷ is selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: halogen, —OR¹⁶, —N(R¹⁶)₂, —C(O)R¹⁶, —C(O)N(R¹⁶)₂, —NO₂, ═O, ═S, ═N(R¹⁶), and —CN.

In some embodiments, for a compound or salt of Formula (I), two of R⁵, R⁶, and R⁷ are hydrogen, and the other of R⁵, R⁶, and R⁷ is selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂. —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle. In some embodiments, two of R⁵, R⁶, and R⁷ are hydrogen, and the other of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₁₋₆ alkyl, C₂₋₆ alkenyl, C₂₋₆ alkynyl, each of which is optionally substituted with one or more substituents independently selected from halogen, —OR¹⁶, —N(R¹⁶)₂, —C(O)R¹⁶, —C(O)N(R¹⁶)₂, —NO₂, ═O, ═S, ═N(R¹⁶), —CN, C₃₋₁₂ carbocycle, and 3- to 12-membered heterocycle.

In some embodiments, for a compound or salt of Formula (I), two of R⁵, R⁶, and R⁷ are hydrogen, and the other of R⁵, R⁶, and R⁷ is selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle. In some embodiments, two of R⁵, R⁶, and R⁷ are hydrogen, and the other of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₃₋₁₂ carbocycle and 3- to 12-membered heterocycle each of which is optionally substituted with one or more substituents independently selected from halogen, —OR¹⁶, —N(R¹⁶)₂, —C(O)R¹⁶, —C(O)N(R¹⁶)₂, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle.

In some embodiments, for a compound or salt of Formula (I), R⁵ and R⁶ are each hydrogen and R⁷ is selected from C₁₋₆ alkyl optionally substituted with one or more substituents independently selected from:
—OR¹⁶, —N(R¹⁶)₂, —C(O)R¹⁶, —NO₂, ═O, —CN; and
C₃₋₆ carbocycle and 5- to 6-membered heterocycle optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

In some embodiments, for a compound or salt of Formula (I), R⁵ and R⁶ are each hydrogen and R⁷ is selected from C₁₋₆ alkyl optionally substituted with one or more substituents independently selected from: —OR¹⁶, —N(R¹⁶)₂, —C(O)R¹⁶, —NO₂, ═O, and —CN. In some embodiments. R⁵ and R⁶ are each hydrogen and R⁷ is selected from C₁₋₆ alkyl optionally substituted with one or more substituents independently selected from: —OR¹⁶, and R¹⁶ is selected from hydrogen and —O—C₁₋₆ alkyl.

In some embodiments, for a compound or salt of Formula (I), R⁵ and R⁶ are each hydrogen and R⁷ is selected from: methyl,

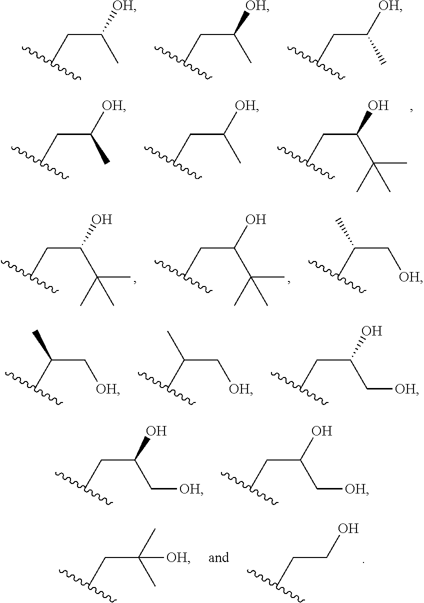

In some embodiments, R⁵ and R⁶ are each hydrogen and R⁷ is selected from: methyl,

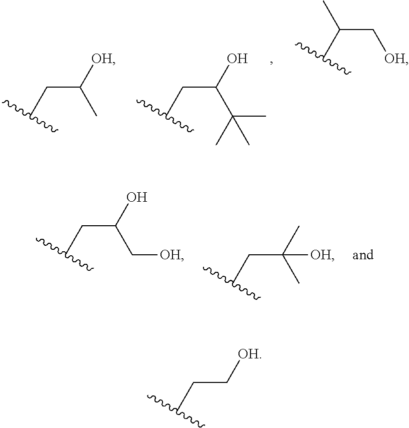

In some embodiments, for a compound or salt of Formula (I), R⁷ is selected from: methyl,

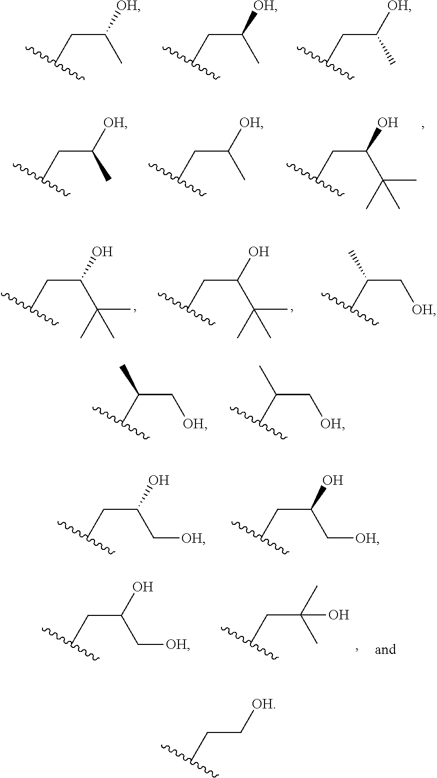

In some embodiments, R⁷ is selected from: methyl,

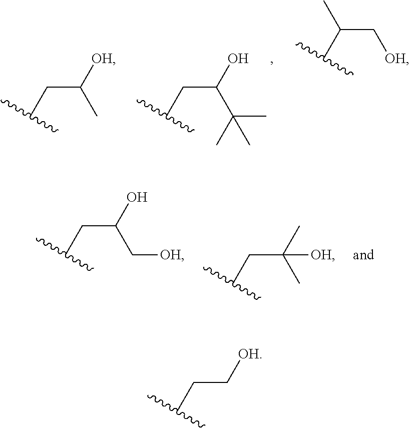

In some embodiments, for a compound or salt of Formula (I), R⁷ is C₁₋₆ alkyl optionally substituted with one or more substituents independently selected from saturated C₃₋₆ carbocycle and 5-membered heteroaryl, each of which is optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN. In some embodiments, R⁷ is C₁₋₆ alkyl optionally substituted with one or more substituents independently selected from saturated C₃₋₆ carbocycle optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN. In some embodiments, R⁷ is C₁₋₆ alkyl optionally substituted with one or more substituents independently selected from 5- to 6-membered heteroaryl optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

In some embodiments, for a compound or salt of Formula (I), R⁷ is C₁₋₆ alkyl optionally substituted with one or more substituents independently selected from cyclopropyl, cyclobutyl, cyclopentyl, cyclohexyl, pyrrole, pyrazole, imidazole, triazole, tetrazole, pyridine, pyrimidine, pyrazine, and triazine, each of which is optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN. In some embodiments, R⁷ is C₁₋₆ alkyl optionally substituted with one or more substituents independently selected from cyclopropyl, pyrazole and triazole, each of which is optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

In some embodiments, for a compound or salt of Formula (I), R⁵ and R⁶ are each hydrogen and R⁷ is selected from:

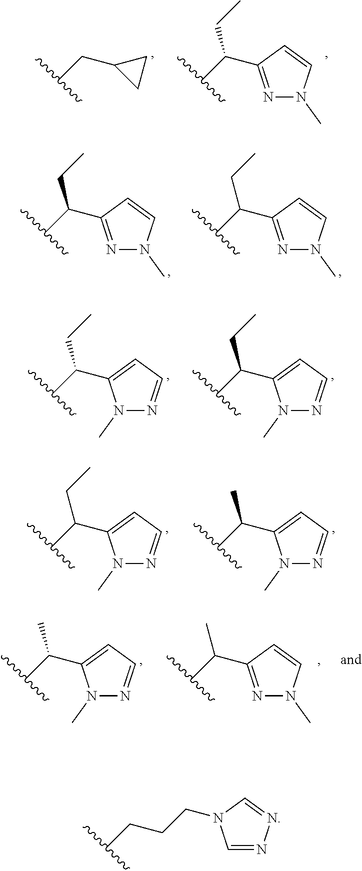

In some embodiments, R⁵ and R⁶ are each hydrogen and R⁷ is selected from:

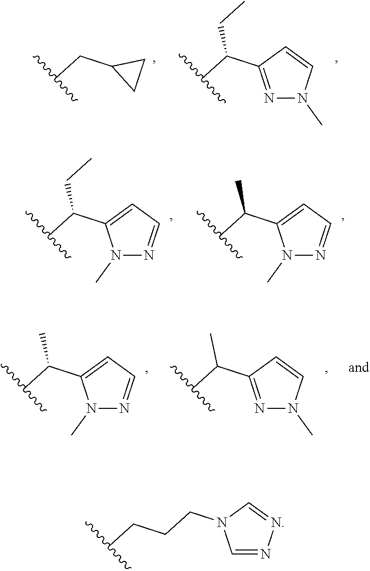

In some embodiments, R⁵ and R⁶ are each hydrogen and R⁷ is selected from:

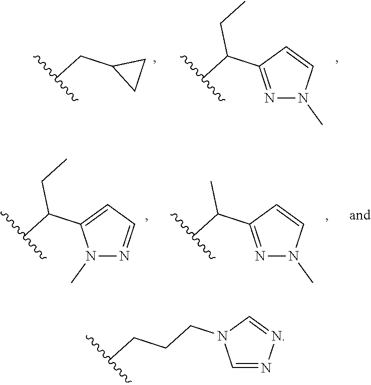

In some embodiments, for a compound or salt of Formula (I). R⁷ is selected from:

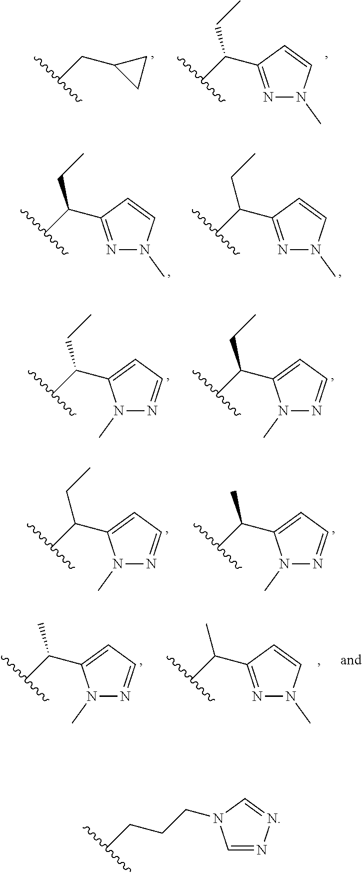

In some embodiments, R⁷ is selected from:

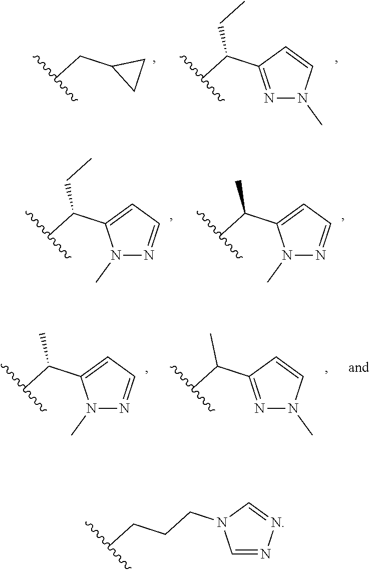

In some embodiments, R⁷ is selected from:

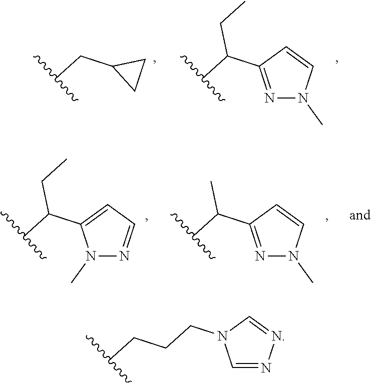

In some embodiments, the compound of Formula (I) is selected from:

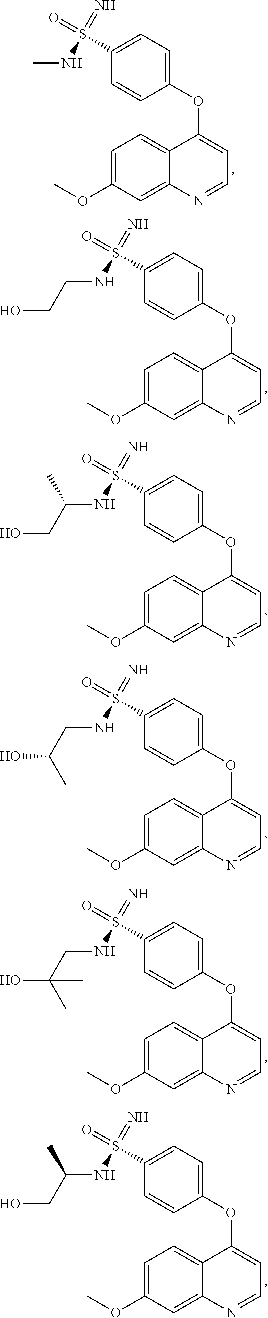

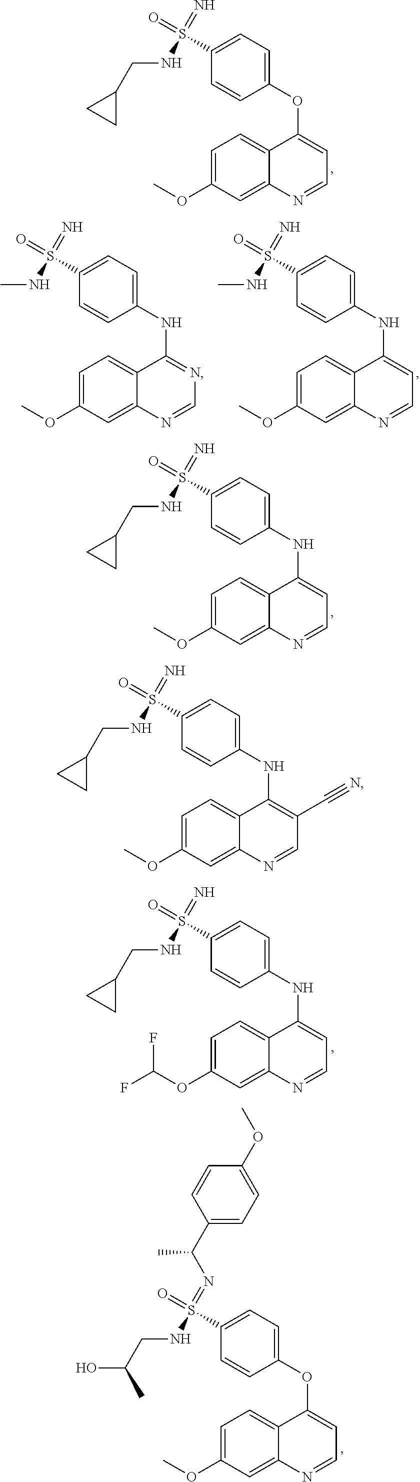

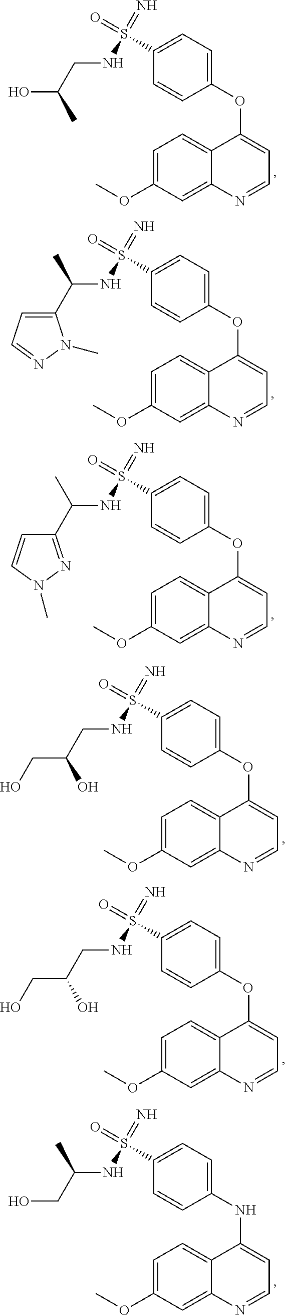

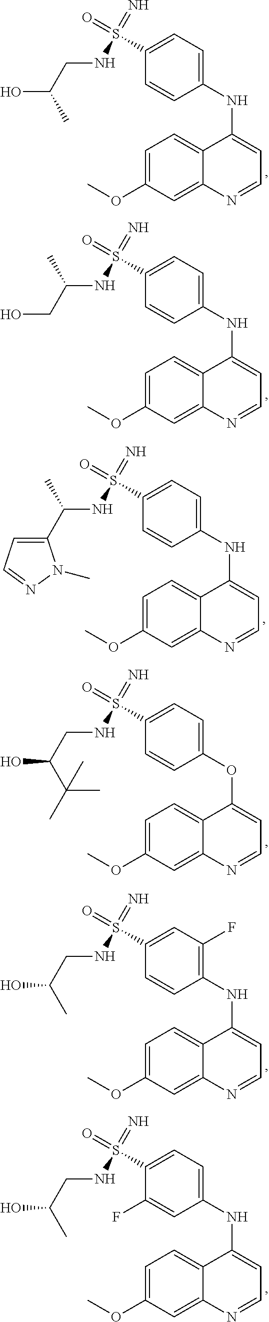

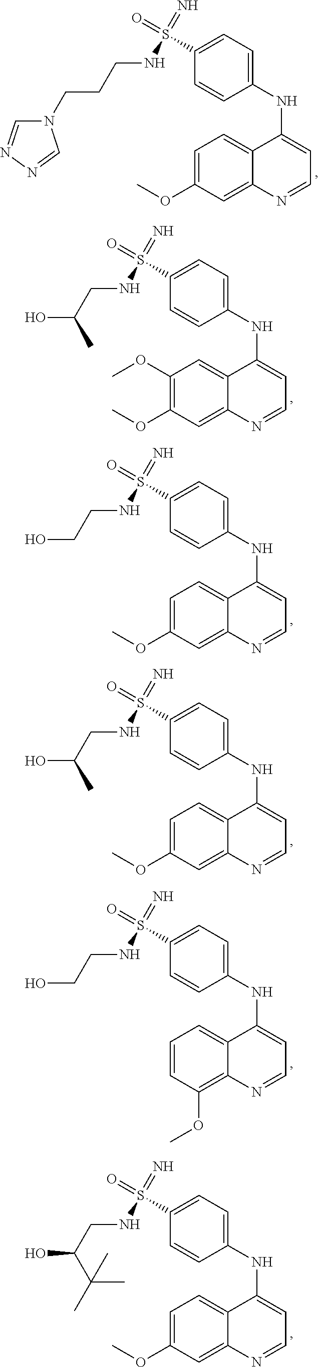

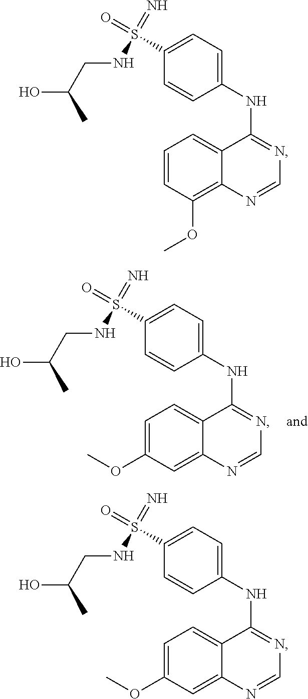

or a pharmaceutically acceptable salt of any one thereof.

In some embodiments, the compound of Formula (I) is selected from:

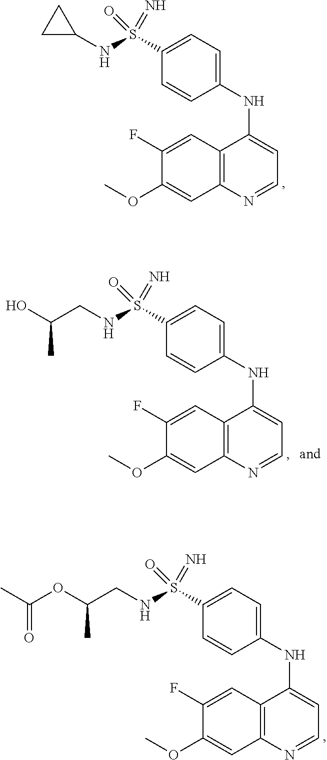 [SMILES: COc1cc2nccc(Nc3ccc([S@@](=N)(=O)NC4CC4)cc3)c2cc1F ; COc1cc2nccc(Nc3ccc([S@@](=N)(=O)NC[C@@H](C)O)cc3)c2cc1F ; COc1cc2nccc(Nc3ccc([S@@](=N)(=O)NC[C@@H](C)OC(C)=O)cc3)c2cc1F]

or a pharmaceutically acceptable salt thereof.

In some aspects the present disclosure provides a compound represented by the structure of Formula (II):

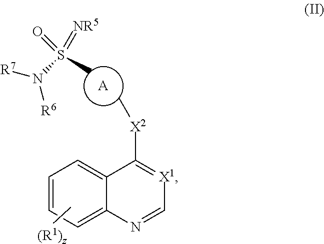

X¹ is selected from N and C(R²);
X² is selected from O, S, C(R³)₂, and N(R⁴);
R¹ is selected from:
   halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, —CN; and
   C₁₋₆ alkyl, C₂₋₆ alkenyl, C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle;
R² is selected from:
   hydrogen, halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, —CN; and
   C₁₋₆ alkyl, C₂₋₆ alkenyl, C₂₋₆ alkynyl, each of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle;
each R³ is independently selected from:
   hydrogen, halogen, —OR¹³, —SR¹³, —N(R¹³)₂, —NO₂, and —CN; and
   C₁₋₆ alkyl optionally substituted with one or more substituents independently selected from halogen, —OR¹³, —SR¹³, —N(R¹³)₂, —NO₂, and —CN;
R⁴ is selected from hydrogen, C₁₋₆ alkyl, and C₃₋₆ carbocycle, wherein C₁₋₆ alkyl and C₃₋₆ carbocycle are each optionally substituted with one or more substituents independently selected from:
   halogen, —OR¹⁴, —SR¹⁴, —N(R¹⁴)₂, —NO₂, and —CN;

Ring A is selected from an optionally substituted C₃₋₁₀ carbocycle and optionally substituted 3- to 10-membered heterocycle wherein substituents on Ring A are independently selected at each occurrence from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
   C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN;
R⁵, R⁶, and R⁷ are each independently selected from hydrogen, optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from:
   halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN;
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle; and
   C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle;
each R¹¹, R¹², R¹³, R¹⁴, R¹⁵, and R¹⁶ is independently selected from:
   hydrogen; and
   C₁₋₆ alkyl, C₂₋₆ alkenyl, C₂₋₆ alkynyl, C₃₋₈ carbocycle, and 3- to 8-membered heterocycle, each of which is optionally substituted with one or more substituents independently selected from halogen, —OH, —CN, —NO₂, —NH₂, ═O, ═S, —C₁₋₆ haloalkyl, and —O—C₁₋₆ alkyl; and
z is selected from 0-3.

In some embodiments, for a compound or salt of Formula (II), z is selected from 0-2. In some embodiments, z is selected from 0 and 1. In some embodiments, z is selected from 1-3. In some embodiments, z is selected from 2 and 3. In some embodiments, z is selected from 1 and 2. In some embodiments, z is 0. In some embodiments, z is 1. In some embodiments, z is 2. In some embodiments, z is 3.

In some embodiments, for a compound or salt of Formula (II), X¹ is selected from N and C(R²), wherein R² is selected from hydrogen, halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, and —CN. In some embodiments, X¹ is selected from N and C(R²), wherein R² is selected from hydrogen, halogen, —OR¹², —N(R¹²)₂, —NO₂, and —CN. In some embodiments, X¹ is selected from N and C(R²), wherein R² is selected from hydrogen and —CN. In some embodiments, X¹ is selected from N, C(H), and C(CN).

In some embodiments, for a compound or salt of Formula (II), X¹ is selected from N and C(R²), wherein R² is selected from C₁₋₆ alkyl, C₂₋₆ alkenyl, C₂₋₆ alkynyl, each of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle. In some embodiments, X¹ is selected from N and C(R²), wherein R² is selected from C₁₋₆ alkyl optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle. In some embodiments, X¹ is selected from N and C(R²), wherein R² is selected from C₁₋₆ alkyl optionally substituted with one or more substituents independently selected from halogen, —OR¹², —N(R¹²)₂, —C(O)R¹², —NO₂, ═O, —CN, C₃₋₆ carbocycle, and 3- to 6-membered heterocycle. In some embodiments, X¹ is selected from N and C(R²), wherein R² is selected from C₂₋₆ alkenyl and C₂₋₆ alkynyl, each of which is optionally substituted with one or more substituents independently selected from halogen, —OR¹², —SR¹², —N(R¹²)₂, —C(O)R¹², —C(O)N(R¹²)₂,
N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle.

In some embodiments, for a compound or salt of Formula (II). X² is selected from O and S. In some embodiments, X² is O. In some embodiments, X² is selected from C(R³), wherein R³ is selected from hydrogen, halogen, —OR¹³, —SR¹³, —N(R¹³)₂, —NO₂, and —CN. In some embodiments, X² is selected from C(R³), wherein R³ is C₁₋₆ alkyl optionally substituted with one or more substituents independently selected from halogen, —OR¹³, —SR¹³, —N(R¹³)₂, —NO₂, and —CN. In some embodiments. X² is selected from N(R⁴), wherein R⁴ is hydrogen. In some embodiments, X² is selected from N(R⁴), wherein R⁴ is selected from C₁₋₆ alkyl and C₃₋₆ carbocycle each of which is optionally substituted with one or more substituents independently selected from: halogen, —OR¹⁴, —SR¹⁴, —N(R¹⁴)₂, —NO₂, and —CN. In some embodiments, X² is N(H). In some embodiments, X² is selected from O and N(H).

In some embodiments, for a compound or salt of Formula (II). R¹ is selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹. —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, and —CN. In some embodiments. R¹ is selected from halogen, —OR¹¹, —N(R¹¹)₂, —C(O)R¹¹, —C(O)N(R¹¹)₂, —NO₂, and —CN.

In some embodiments, for a compound or salt of Formula (II), R¹ is selected from C₁₋₆ alkyl, C₂₋₆ alkenyl, C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle. In some embodiments, R¹ is selected from C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle. In some embodiments, R¹ is selected from C₂₋₆ alkenyl and C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle.

In some embodiments, for a compound or salt of Formula (II). R¹ is selected from: halogen, —OR¹¹, —N(R¹¹)₂, —C(O)R¹¹, —NO₂, —CN, and C₁₋₆ alkyl optionally substituted with one or more substituents independently selected from halogen, —OR¹¹, —N(R¹¹)₂, —C(O)R¹¹, —NO₂, and —CN. In some embodiments, R¹ is selected from —OR¹¹ and R¹¹ is selected from C₁₋₆ alkyl and C₁₋₆ haloalkyl. In some embodiments, R¹ is selected from —OR¹¹ and R¹¹ is selected from C₁₋₆ alkyl. In some embodiments. R¹ is selected from —OR¹¹ and R¹¹ is selected from C₁₋₆ haloalkyl. In some embodiments. R¹ is selected from methoxy and —OC(H) F₂. In some embodiments, R¹ is methoxy. In some embodiments. R¹ is —OC(H) F₂.

In some embodiments, for a compound or salt of Formula (II). Ring A is C₃₋₁₀ carbocycle optionally substituted with one or more substituents independently selected from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵. —OC(O)R¹⁵, —S(O)R¹⁵, —S(O)₂R¹⁵, —NO₂, ═O, —CN, and C₁₋₆ haloalkyl; and
C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵—C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (II). Ring A is C₃₋₁₀ carbocycle optionally substituted with one or more substituents independently selected from: halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl. In some embodiments, Ring A is C₃₋₁₀ carbocycle optionally substituted with one or more substituents independently selected from C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (II). Ring A is selected from optionally substituted C₃₋₉ carbocycle, optionally substituted C₃₋₈ carbocycle, optionally substituted C₃₋₇ carbocycle, optionally substituted C₃₋₆ carbocycle, optionally substituted C₃₋₅ carbocycle, and optionally substituted C₃₋₄ carbocycle. In some embodiments, Ring A is selected from optionally substituted C₄₋₁₀ carbocycle, optionally substituted C₈₋₁₀ carbocycle, optionally substituted C₆₋₁₀ carbocycle, optionally substituted C₇₋₁₀ carbocycle, optionally substituted C₈₋₁₀ carbocycle, and optionally substituted C₉₋₁₀ carbocycle. In some embodiments, Ring A is selected from optionally substituted Ca carbocycle, optionally substituted C₄ carbocycle, optionally substituted C₅ carbocycle, optionally substituted C₆ carbocycle, optionally substituted C₇ carbocycle, optionally substituted C₈ carbocycle, optionally substituted C₉ carbocycle, and optionally substituted C₁₀ carbocycle. In some embodiments, Ring A is selected from an optionally substituted saturated C₃₋₁₀ carbocycle and optionally substituted unsaturated C₃₋₁₀ carbocycle.

In some embodiments, for a compound or salt of Formula (II). Ring A is C₃₋₆ carbocycle optionally substituted with one or more substituents independently selected from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵—C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (II). Ring A is C₃₋₆ carbocycle optionally substituted with one or more substituents independently selected from: halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl. In some embodiments, Ring A is C₃₋₁₀ carbocycle optionally substituted with one or more substituents independently selected from C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (II). Ring A is selected from C₃₋₆ carbocycle optionally substituted with one or more substituents independently selected from: halogen, —OR¹⁵, —N(R¹⁵)₂, —C(O)R¹⁵, —NO₂, ═O, —CN, and C₁₋₆ haloalkyl. In some embodiments, Ring A is selected from cyclopropyl, cyclobutyl, cyclopentyl, cyclohexyl, and phenyl, each of which is optionally substituted with one or more substituents independently selected from: halogen, —OR¹⁵, —N(R¹⁵)₂, —C(O)R¹⁵, —NO₂, ═O, —CN, and C₁₋₆ haloalkyl. In some embodiments, Ring A is phenyl optionally substituted with one or more substituents independently selected from: halogen, —OR¹⁵, —N(R¹⁵)₂, —C(O)R¹⁵, —NO₂, ═O, —CN, and C₁₋₆ haloalkyl. In some embodiments, Ring A is phenyl optionally substituted with one or more substituents independently selected from: fluorine, chlorine. CN, and C₁₋₆ haloalkyl. In some embodiments, Ring A is phenyl optionally substituted with fluorine.

In some embodiments, for a compound or salt of Formula (II), Ring A is 3- to 10-membered heterocycle optionally substituted with one or more substituents independently selected from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵—C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (II). Ring A is 3- to 10-membered heterocycle optionally substituted with one or more substituents independently selected from: halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵. —S(O)R¹⁵, —S(O)₂R¹⁵, —NO₂, ═O, —CN, and C₁₋₆ haloalkyl. In some embodiments, Ring A is 3- to 10-membered heterocycle optionally substituted with one or more substituents independently selected from: C₁₋₆ alkyl, C₂₋₆ alkenyl, C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN.

In some embodiments, for a compound or salt of Formula (II). Ring A is an optionally substituted 3- to 10-membered heterocycle comprising at least one heteroatom selected from nitrogen, oxygen, and sulfur. In some embodiments, Ring A is selected from an optionally substituted 3- to 9-membered heterocycle, an optionally substituted 3- to 8-membered heterocycle, an optionally substituted 3- to 7-membered heterocycle, an optionally substituted 3- to 6-membered heterocycle, an optionally substituted 3- to 5-membered heterocycle, and an optionally substituted 3- to 4-membered heterocycle. In some embodiments, Ring A is selected from an optionally substituted 4- to 10-membered heterocycle, an optionally substituted 5- to 10-membered heterocycle, an optionally substituted 6- to 10-membered heterocycle, an optionally substituted 7- to 10-membered heterocycle, an optionally substituted 8- to 10-membered heterocycle, and an optionally substituted 9- to 10-membered heterocycle. In some embodiments, Ring A selected from is an optionally substituted 4- to 10-membered saturated heterocycle and optionally substituted 4- to 10-membered unsaturated heterocycle.

In some embodiments, for a compound or salt of Formula (II), wherein two of R⁵, R⁶, and R⁷ are selected from hydrogen and the other of R⁵, R⁶, and R⁷ is selected from C₁₋₆ alkyl optionally substituted with one or more substituents independently selected from:
—OR¹⁶, —N(R¹⁶)₂, —C(O)R¹⁶, —NO₂, ═O, —CN; and
C₃₋₆ carbocycle and 5- to 6-membered heterocycle optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

In some embodiments, for a compound or salt of Formula (II), at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from:
halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN;
C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle; and
C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶—NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle.

In some embodiments, for a compound or salt of Formula (II), at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), and —CN. In some embodiments, at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: halogen, —OR¹⁶, —N(R¹⁶)₂, —C(O)R¹⁶, —C(O)N(R¹⁶)₂, —NO₂, ═O, ═S, ═N(R¹⁶), and —CN.

In some embodiments, for a compound or salt of Formula (II), at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶—S(NR¹⁶)₂R¹⁶—NO₂, ═O, ═S, ═N(R¹⁶), —CN, C₃₋₁₂ carbocycle, and 3- to 12-membered heterocycle. In some embodiments, at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₁₋₆ alkyl, C₂₋₆ alkenyl, C₂₋₆ alkynyl, each of which is optionally substituted with one or more substituents independently selected from halogen, —OR¹⁶, —N(R¹⁶)₂, —C(O)R¹⁶, —C(O)N(R¹⁶)₂, —NO₂, ═O, ═S, ═N(R¹⁶), —CN, C₃₋₁₂ carbocycle, and 3- to 12-membered heterocycle.

In some embodiments, for a compound or salt of Formula (II), at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle. In some embodiments, at least one of R⁵, R⁶, and R⁷ is hydrogen, and the other two of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R³. R⁶, and R⁷ are independently selected at each occurrence from: C₃₋₁₂ carbocycle and 3- to 12-membered heterocycle each of which is optionally substituted with one or more substituents independently selected from halogen, —OR¹⁶, —N(R¹⁶)₂, —C(O)R¹⁶, —C(O)N(R¹⁶)₂, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle.

In some embodiments, for a compound or salt of Formula (II), two of R⁵, R⁶, and R⁷ are hydrogen, and the other of R⁵, R⁶, and R⁷ is selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from:
halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN;
C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle; and
C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle.

In some embodiments, for a compound or salt of Formula (II), two of R⁵, R⁶, and R⁷ are hydrogen, and the other of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), and —CN. In some embodiments, two of R⁵, R⁶, and R⁷ are hydrogen, and the other R⁵, R⁶, and R⁷ is selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: halogen, —OR¹⁶, —N(R¹⁶)₂, —C(O)R¹⁶, —C(O)N(R¹⁶)₂, —NO₂, ═O, ═S, ═N(R¹⁶), and —CN.

In some embodiments, for a compound or salt of Formula (II), two of R⁵, R⁶, and R⁷ are hydrogen, and the other of R⁵, R⁶, and R⁷ is selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂. —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶—S(NR¹⁶)₂R¹⁶—NO₂, ═O, ═S, ═N(R¹⁶), —CN, C₃₋₁₂ carbocycle, and 3- to 12-membered heterocycle. In some embodiments, two of R⁵, R⁶, and R⁷ are hydrogen, and the other of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₁₋₆ alkyl, C₂₋₆ alkenyl, C₂₋₆ alkynyl, each of which is optionally substituted with one or more substituents independently selected from halogen, —OR¹⁶, —N(R¹⁶)₂, —C(O)R¹⁶, —C(O)N(R¹⁶)₂, —NO₂, ═O, ═S, ═N(R¹⁶), —CN, C₃₋₁₂ carbocycle, and 3- to 12-membered heterocycle.

In some embodiments, for a compound or salt of Formula (II), two of R⁵, R⁶, and R⁷ are hydrogen, and the other of R⁵, R⁶, and R⁷ is selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle. In some embodiments, two of R⁵, R⁶, and R⁷ are hydrogen, and the other of R⁵, R⁶, and R⁷ are selected from optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from: C₃₋₁₂ carbocycle and 3- to 12-membered heterocycle each of which is optionally substituted with one or more substituents independently selected from halogen, —OR¹⁶, —N(R¹⁶)₂, —C(O)R¹⁶, —C(O)N(R¹⁶)₂, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle.

In some embodiments, for a compound or salt of Formula (II), R⁵ and R⁶ are each hydrogen and R⁷ is selected from C₁₋₆ alkyl optionally substituted with one or more substituents independently selected from:
—OR¹⁶, —N(R¹⁶)₂, —C(O)R¹⁶, —NO₂, ═O, —CN; and
C₃₋₆ carbocycle and 5- to 6-membered heterocycle optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

In some embodiments, for a compound or salt of Formula (II), R⁵ and R⁶ are each hydrogen and R⁷ is selected from C₁₋₆ alkyl optionally substituted with one or more substituents independently selected from: —OR¹⁶, —N(R¹⁶)₂, —C(O)R¹⁶, —NO₂, ═O, and —CN. In some embodiments. R⁵ and R⁶ are each hydrogen and R⁷ is selected from C₁₋₆ alkyl optionally substituted with one or more substituents independently selected from: —OR¹⁶, and R¹⁶ is selected from hydrogen and —O—C₁₋₆ alkyl.

In some embodiments, for a compound or salt of Formula (II), R⁵ and R⁶ are each hydrogen and R⁷ is selected from: methyl,

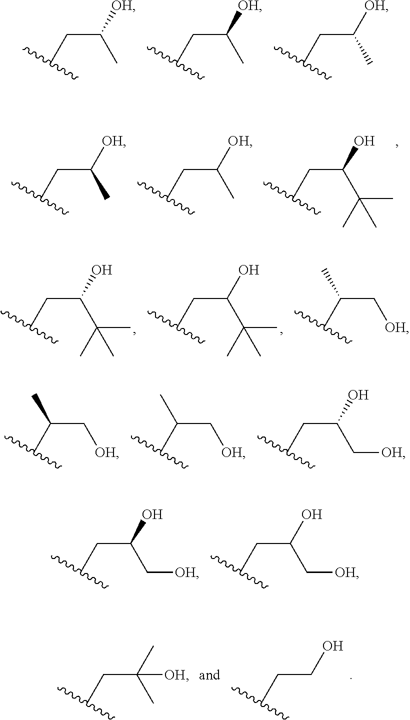

In some embodiments, R⁵ and R⁶ are each hydrogen and R⁷ is selected from: methyl,

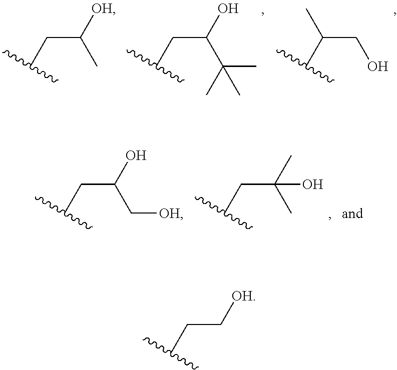

In some embodiments, for a compound or salt of Formula (II), R⁷ is selected from: methyl,

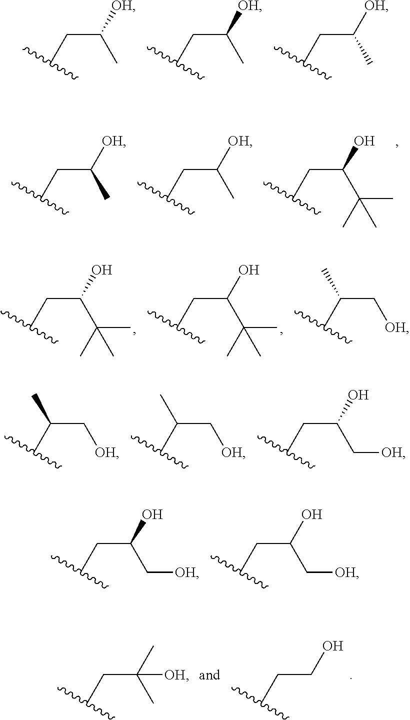

In some embodiments, R⁷ is selected from: methyl,

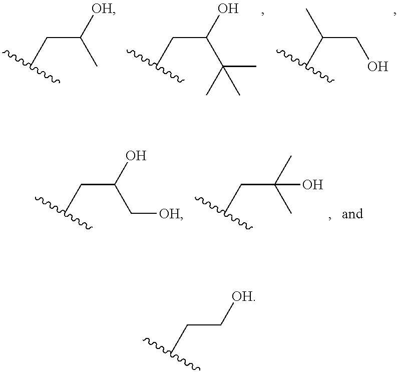

In some embodiments, for a compound or salt of Formula (II), R⁷ is C₁₋₆ alkyl optionally substituted with one or more substituents independently selected from saturated C₃₋₆ carbocycle and 5-membered heteroaryl, each of which is optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN. In some embodiments, R⁷ is C₁₋₆ alkyl optionally substituted with one or more substituents independently selected from saturated C₃₋₆ carbocycle optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN. In some embodiments, R⁷ is C₁₋₆ alkyl optionally substituted with one or more substituents independently selected from 5- to 6-membered heteroaryl optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

In some embodiments, for a compound or salt of Formula (II), R⁷ is C₁₋₆ alkyl optionally substituted with one or more substituents independently selected from cyclopropyl, cyclobutyl, cyclopentyl, cyclohexyl, pyrrole, pyrazole, imidazole, triazole, tetrazole, pyridine, pyrimidine, pyrazine, and triazine, each of which is optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN. In some embodiments, R⁷ is C₁₋₆ alkyl optionally substituted with one or more substituents independently selected from cyclopropyl, pyrazole and triazole, each of which is optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

In some embodiments, for a compound or salt of Formula (II), R⁵ and R⁶ are each hydrogen and R⁷ is selected from:

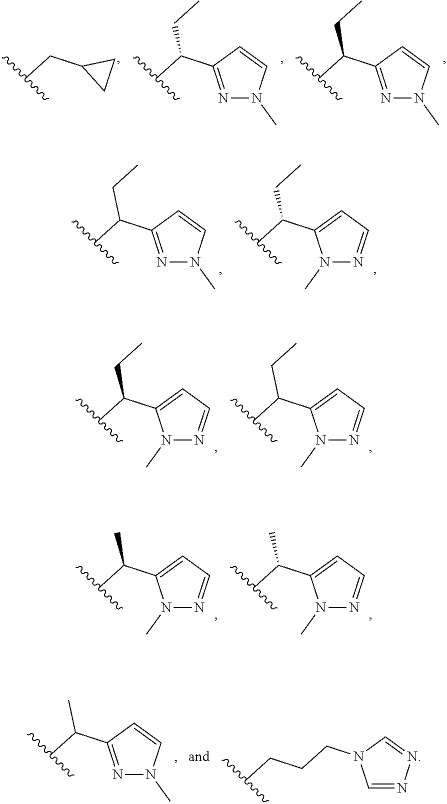

In some embodiments, R⁵ and R⁶ are each hydrogen and R⁷ is selected from:

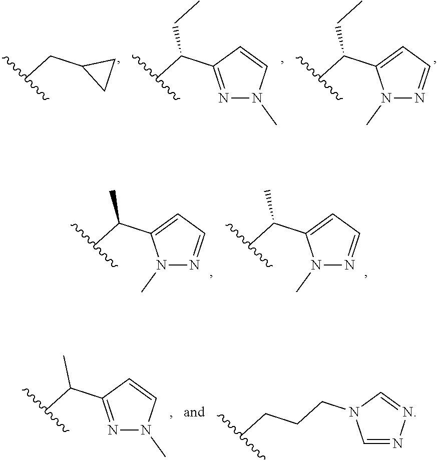

In some embodiments, R⁵ and R⁶ are each hydrogen and R⁷ is selected from:

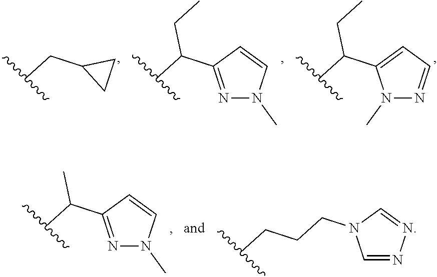

In some embodiments, for a compound or salt of Formula (II), R⁷ is selected from:

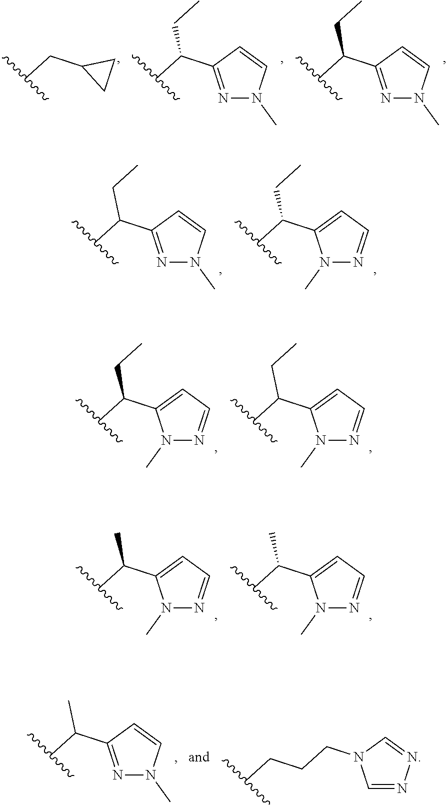

In some embodiments, R⁷ is selected from:

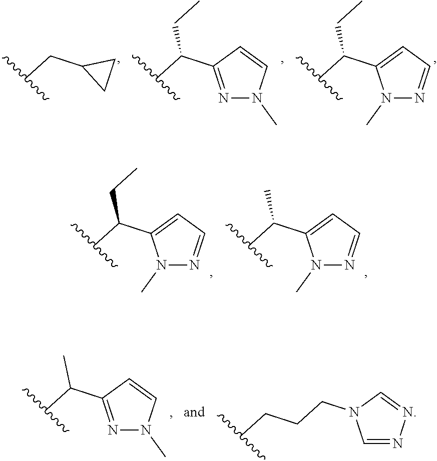

In some embodiments, R⁷ is selected from:

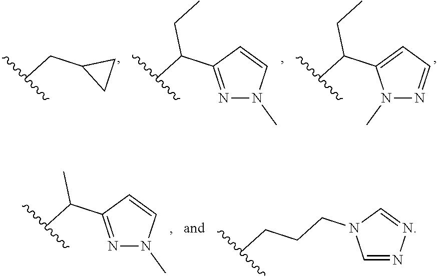

In some embodiments, the compound of Formula (II) is selected from:

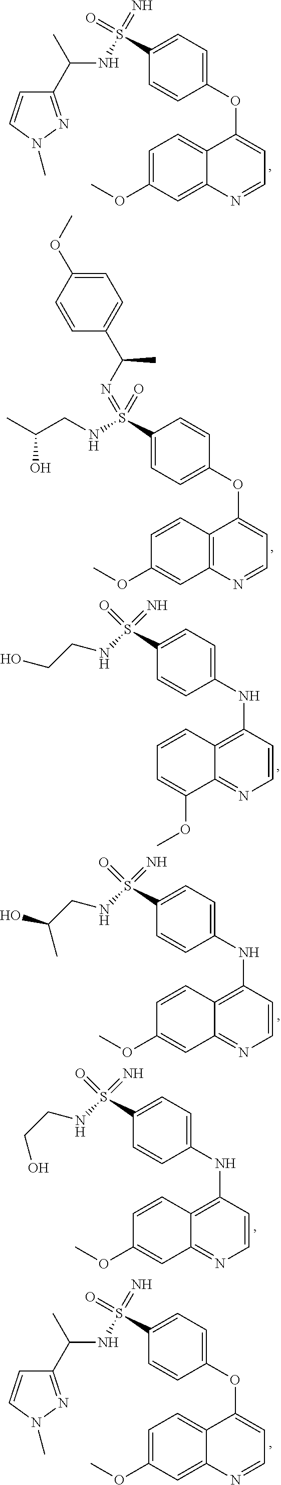

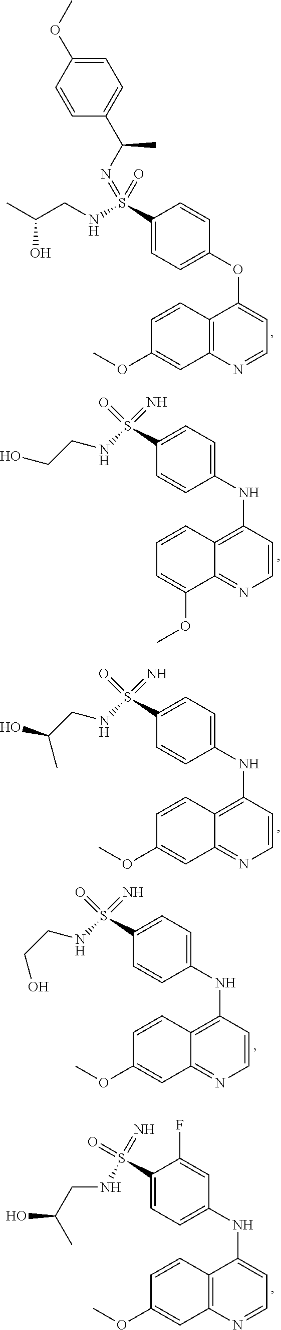

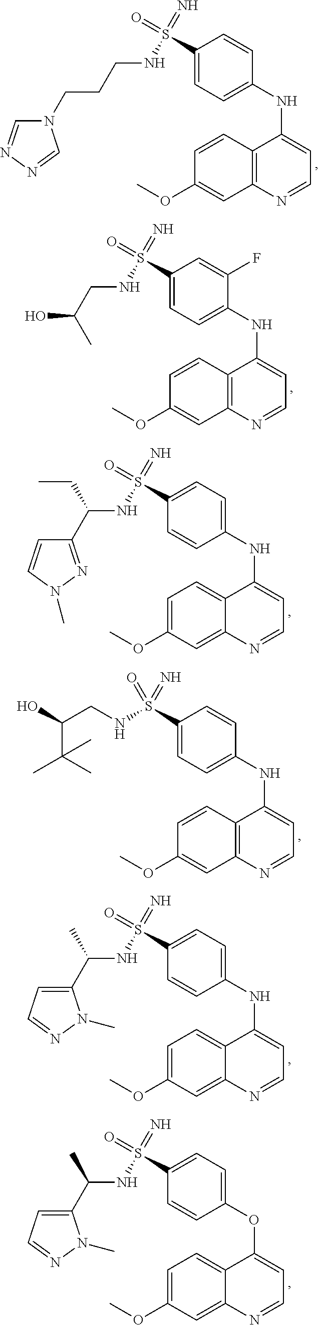

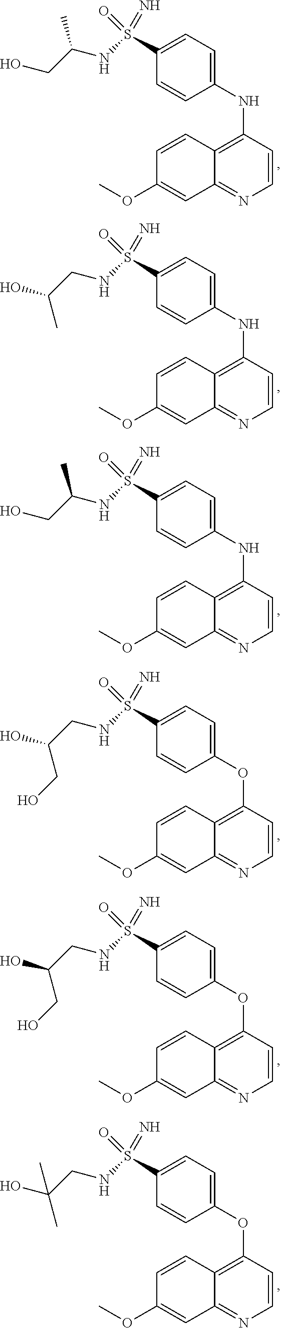

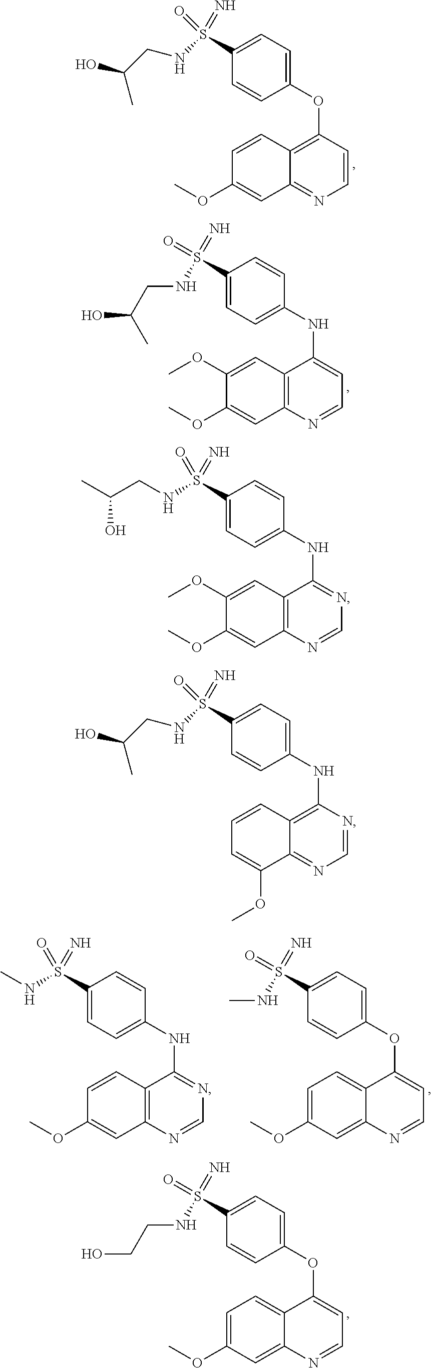

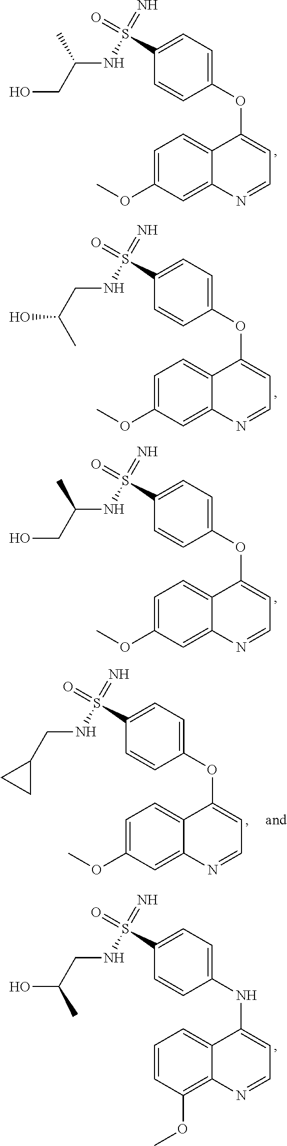

or a pharmaceutically acceptable salt thereof.

In some embodiments, the disclosure provides a compound selected from:

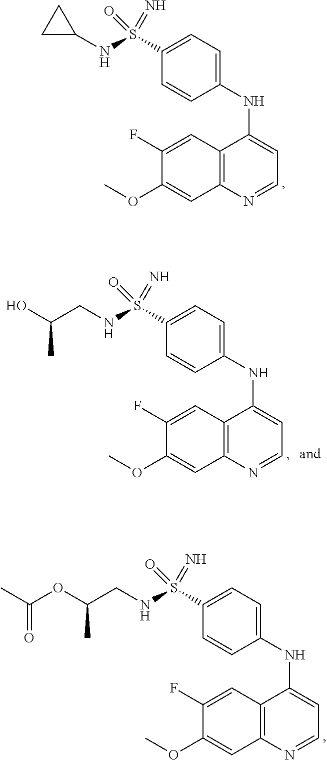 [SMILES: COc1cc2nccc(Nc3ccc([S@@](=N)(=O)NC4CC4)cc3)c2cc1F ; COc1cc2nccc(Nc3ccc([S@@](=N)(=O)NC[C@@H](C)O)cc3)c2cc1F ; COc1cc2nccc(Nc3ccc([S@@](=N)(=O)NC[C@@H](C)OC(C)=O)cc3)c2cc1F]

or a pharmaceutically acceptable salt thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formula (I) or (II), are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (±) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or(S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (I) or (II), herein may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof. (Jean Jacques. Andre Collet. Samuel H. Wilen. “Enantiomers. Racemates and Resolutions”. John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formula (I) or (II), may comprise two or more enantiomers or diatereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds. (1962) by E. L. Eliel. McGraw Hill; Lochmuller (1975)J. Chromatogr., 113 (3); 283-302). Racemic mixtures of chiral compounds can be separated and isolated by any suitable method, including, but not limited to: (1) formation of ionic, diastereomeric salts with chiral compounds and separation by fractional crystallization or other methods. (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (I) or (II), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

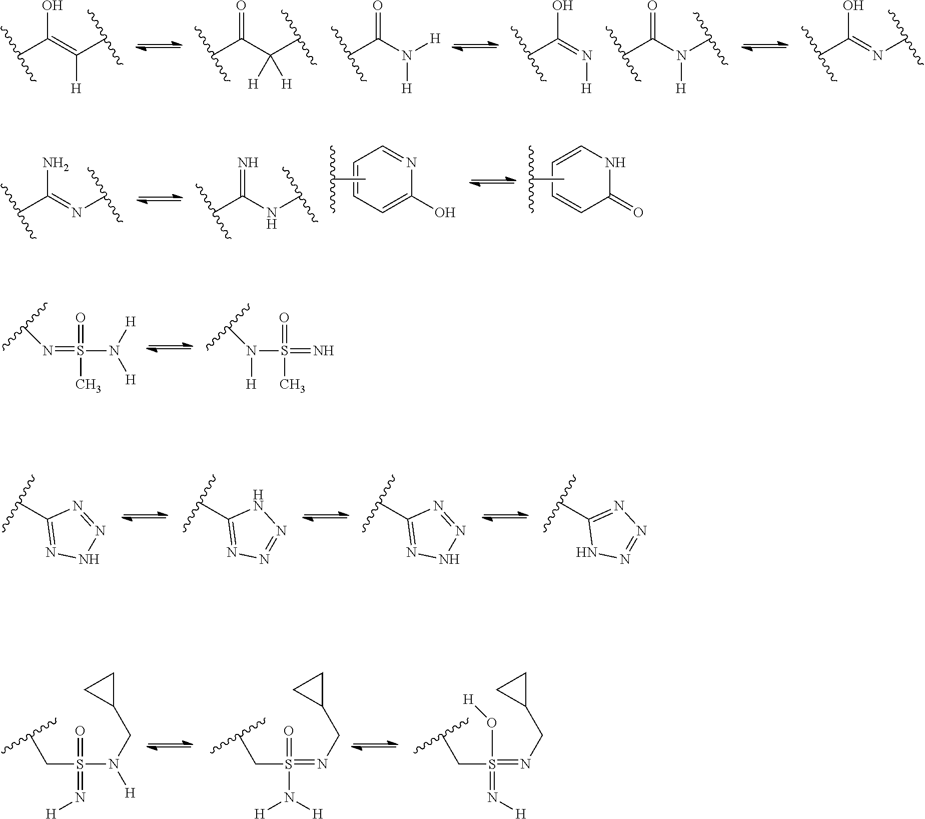

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In; Curr., Pharm. Des., 2000; 6 (10)] 2000, 110 pp; George W; Varma, Rajender S. The Synthesis of Radiolabeled Compounds via Organometallic Intermediates, Tetrahedron, 1989, 45 (21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (I) or (II). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide, trifluoracetic acid, or formic acid.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formula (I) or (II), and at least one pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

A compound or salt of any one of Formula (I) or (II), may be formulated in any suitable pharmaceutical formulation. A pharmaceutical formulation of the present disclosure typically contains an active ingredient (e.g., compound or salt of any one of Formula (I) or (II)), and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

Pharmaceutical compositions may also be prepared from a compound or salt of any one of Formula (I) or (II), and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson. Philip O.; Knoben, James E.; Troutman, William G, eds., Handbook of Clinical Drug Data, Tenth Edition, McGraw-Hill, 2002: Pratt and Taylor, eds., Principles of Drug Action, Third Edition, Churchill Livingston, New York, 1990; Katzung, ed., Basic and Clinical Pharmacology, Ninth Edition, McGraw Hill, 2003: Goodman and Gilman, eds., The Pharmacological Basis of Therapeutics, Tenth Edition, McGraw Hill, 2001: Remingtons Pharmaceutical Sciences, 20th Ed., Lippincott Williams & Wilkins., 2000; Martindale,The Extra Pharmacopoeia, Thirty-Second Edition (The Pharmaceutical Press, London, 1999).

### Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In some aspects, the present disclosure provides a method for treatment, comprising administering to a subject in need thereof an effective amount of a compound or salt of Formula (I) or (II).

In certain aspects, the present disclosure provides a method for immunotherapeutic treatment to a subject in need thereof. In some embodiments, immunotherapy may be used to treat disorders resulting from a virus, bacteria, cancer, or tumor. In some embodiments, the present disclosure can be used as a method for immunotherapeutic treatment in a subject in need thereof, comprising administering to the subject a pharmaceutical composition comprising a compound or salt of Formula (I) or (II), and a pharmaceutically acceptable excipient. In certain embodiments, a pharmaceutical composition comprising a compound or salt of Formula (I) or (II), and a pharmaceutically acceptable excipient may be used as an immunological adjuvant. In some embodiments, the immunological adjuvant of the present disclosure may be used in combination with a vaccine for the treatment or prevention a disease, state or condition in a patient in need thereof. In some cases, immunological adjuvant may be as described Gutjahr. A., et al. Triggering Intracellular Receptors for Vaccine Adjuvantation. Trends in Immunology, 37 (9), 573-587 (2016).

In certain embodiments, the present disclosure can be used as a method of activating an immune response to a pathogen in a subject in need thereof, comprising administering to the subject a pharmaceutical composition comprising a compound or salt of Formula (I) or (II), and a pharmaceutically acceptable excipient.

In certain embodiments, the present disclosure can be used as a method of inhibiting ENPP1 in a subject in need thereof, comprising administering to the subject a pharmaceutical composition comprising a compound or salt of Formula (I) or (II), and a pharmaceutically acceptable excipient.

In certain embodiments, the present disclosure can be used as a method of activating STING activity in a subject in need thereof, comprising administering to the subject a pharmaceutical composition comprising a compound or salt of Formula (I) or (II), and a pharmaceutically acceptable excipient.

ENPP1 is known in the art to be implicated in tumor progression and immunosuppression in the tumor microenvironment. See Onyedibe, K. I, et al., ENPP1, an Old Enzyme with New Functions, and Small Molecule Inhibitors—A STING in the Tale of ENPP1. Molecules, (24), 4192-4210 (2019). As such, inhibition of ENPP1 in solid tumors is a promising treatment modality. Gangar, M., et al. Design, synthesis and biological evaluation studies of novel small molecule ENPP1 inhibitors for cancer immunotherapy. Bioorganic Chemistry (119), 105549.

Accordingly, in some aspects, the present disclosure provides a method of treating cancer in a subject in need thereof, comprising administering a compound the present disclosure. In some embodiments, the present disclosure provides a method of treating cancer in a subject in need thereof, comprising administering a therapeutically effective amount of a compound the present disclosure.

The compounds described herein can be used in the preparation of medicaments for the treatment of cancer. In addition, the present disclosure provides a method of treating cancer in a subject in need thereof comprising administration of a compound or salt of Formula (I) or (II). In some embodiments, the method comprises administration of a pharmaceutical compositions containing at least one compound described herein, or a pharmaceutically acceptable salt, pharmaceutically acceptable prodrug, or pharmaceutically acceptable solvate thereof, in therapeutically effective amounts to said subject. In another aspect, the present disclosure provides the use of a compound or salt of Formula (I) or (II) for the treatment of cancer, or the use of a compound or salt of Formula (I) or (II) for the manufacture of a medicament for the treatment of cancer.

In some embodiments, the cancer comprises a solid tumor. In some embodiments, the cancer is selected from carcinoma, sarcoma, and lymphoma. In some embodiments, the cancer is selected from: adrenal cancer, liver cancer, kidney cancer, bladder cancer, breast cancer, colon cancer, stomach cancer, ovarian cancer, cervical cancer, uterine cancer, esophageal cancer, colorectal cancer, prostate cancer, pancreatic cancer, lung cancer, thyroid cancer, glioblastoma, melanoma, and head and neck cancer. In some embodiments, the cancer is breast cancer. In some embodiments, the cancer is lymphoma.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-9 show general and exemplary procedures for the preparation of the claimed ENPP1 modulators. Example 10 demonstrates bioassay procedures for the enzymatic inhibition of ENPP1 of selected ENPP1 modulators as described herein.

### Example 1. General Scheme—Synthesis of Compounds (1 and 2)

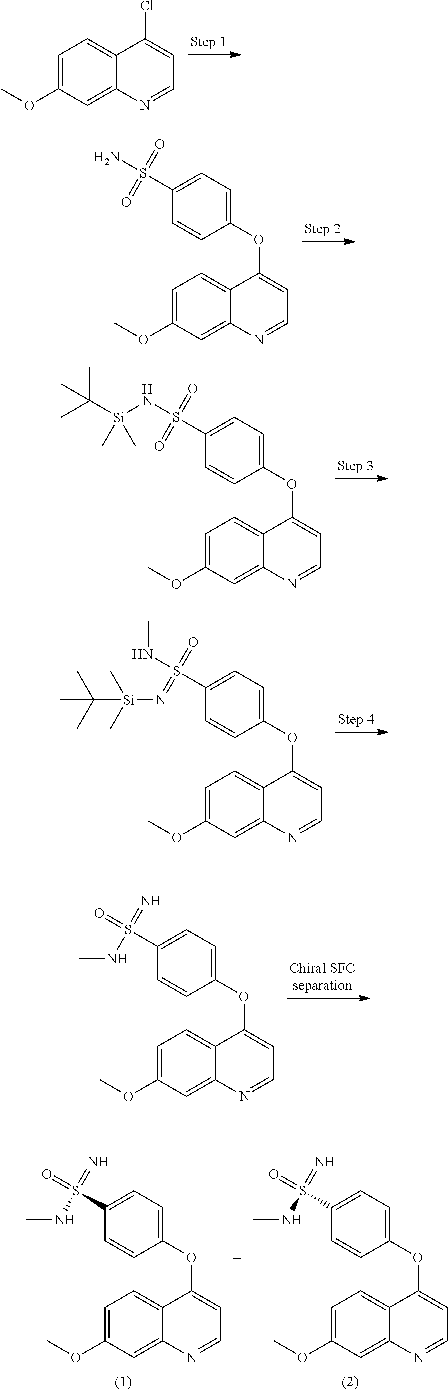

### Example 2. Exemplary Scheme—Synthesis of Compounds (1 and 2)

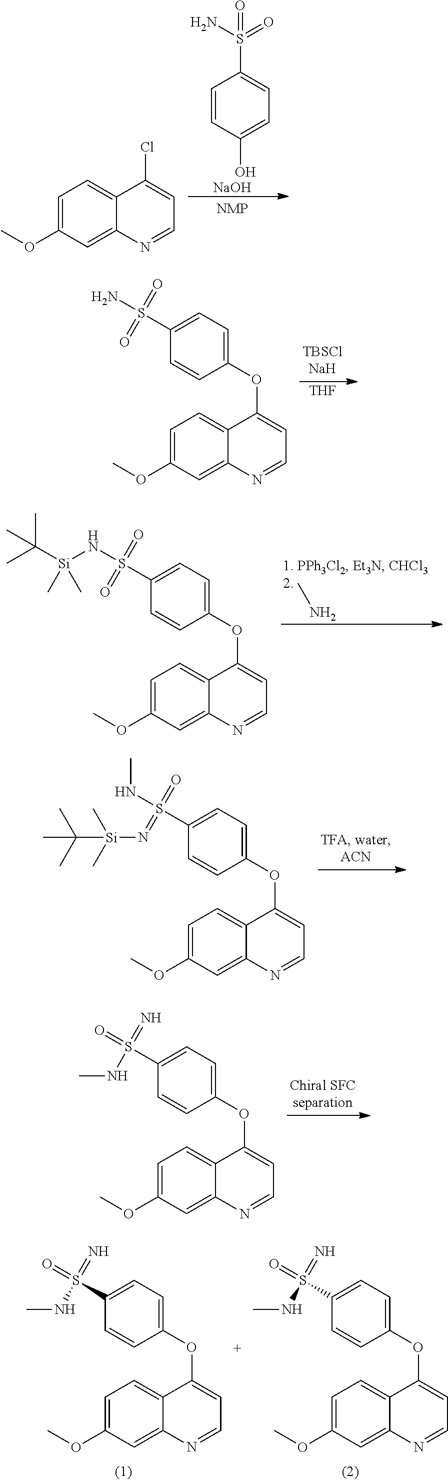

### Step 1: Preparation of 4-((7-methoxyquinolin-4-yl)oxy)benzenesulfonamide

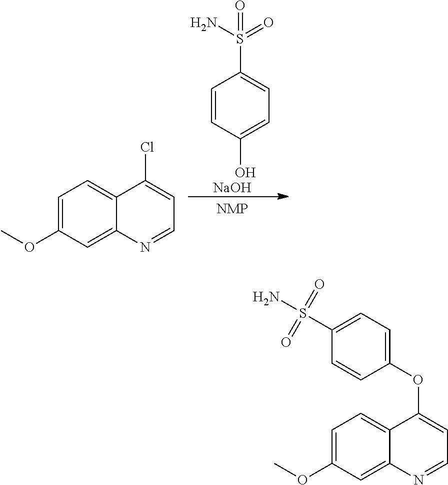

To a stirring solution of 4-hydroxybenzenesulfonamide (5.59 g, 32.3 mmol) in methanol (20 mL) was added 10M aqueous NaOH (2.6 mL, 26 mmol). The mixture was sonicated for 10 minutes then, concentrated and dried under vacuum. To the resulting solid was added 4-chloro-7-methoxyquinoline (5 g, 25.8 mmol) followed by N-methyl-2-pyrrolidone (20 mL). The mixture was heated at 150° C. for 10 h. The mixture was cooled to room temperature and diluted with water (50 ml). The resulting solution was basified to pH˜8 with aqueous 1M K₂HPO₄. The solids were filtered, washed with water, and dried by air filtration. The dried precipitate was suspended in acetonitrile (40 ml), heated to 50° C., and stirred for 15 minutes. The mixture was cooled to room temperature, filtered, and dried under vacuum. 6.4 g, LC-MS: m/z [M+H]⁺ 331.20.

### Step 2: Preparation of N-(tert-butyldimethylsilyl)-4-((7-methoxyquinolin-4-yl)oxy)benzenesulfonamide

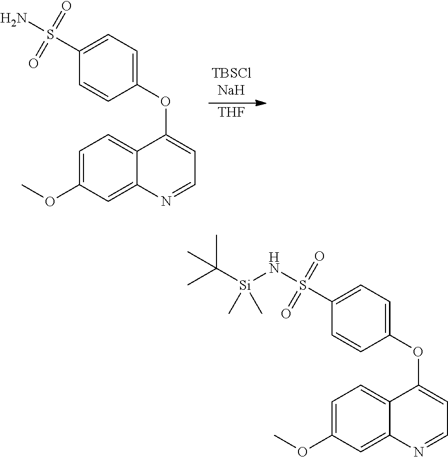 [SMILES: COc1ccc2c(Oc3ccc(S(N)(=O)=O)cc3)ccnc2c1 ; COc1ccc2c(Oc3ccc(S(=O)(=O)N[Si](C)(C)C(C)(C)C)cc3)ccnc2c1 ; "THF"]

To a suspension of 4-((7-methoxyquinolin-4-yl)oxy)benzenesulfonamide (1.8 g, 5.45 mmol) in THF (30 mL) was added portionwise sodium hydride (0.288 g, 11.99 mmol) over 15 minutes at 0° C. After stirring for 15 minutes, a solution of t-butyldimethylchlorosilane (0.985 g, 6.54 mmol) in THF (10 ml) was added dropwise over 10 mins. After stirring for 2 hours, the mixture was carefully quenched with a 2:1 mixture of 1M K₂HPO₄ and 1M KH₂PO₄ (aq, 50 mL). The layers were separated and the organic layer was partially concentrated. The crude mixture was diluted with EtOAc (40 mL) and washed with water (40 mL). The organic layer was dried with anhydrous sodium sulfate, filtered, and concentrated in vacuo. The resulting crude product was slurried in EtOAc:Et₂O (1:1, 30 mL), filtered, and washed. The solids were dried under vacuum to give the title compound. 1.3 g, LC-MS: m/z [M+H]⁺ 445.20

### Step 3: Preparation of 4-((7-methoxyquinolin-4-yl)oxy)-N-methylbenzenesulfonimidamide

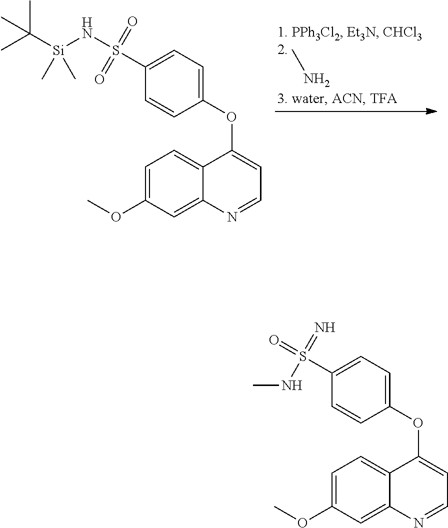

To a solution of N-(tert-butyldimethylsilyl)-4-((7-methoxyquinolin-4-yl)oxy)benzenesulfonamide (6 g, 13.0 mmol) in CHCl₃ (50 mL) was added triethylamine (4.5 mL, 32.5 mmol) followed by dichlorotriphenylphosphorane (4.76 g, 14.3 mmol) at 0° C. The reaction mixture was stirred at 0° C. for 20 minutes. To the reaction was added methylamine in THE solution (800 mg, 25.9 mmol) at 0° C. The reaction mixture was stirred at 0° C. for 2 hr. The reaction mixture was diluted with water (150 mL) and extracted with ethyl acetate (3×80 mL). The combined organic layers were washed with brine and dried over Na₂SO₄. The mixture was filtered and the filtrate was concentrated under reduced pressure. The tert-butyldimethylsilyl was removed under the presence of TFA during HPLC purification. The product was purified by reverse phase HPLC purification (eluent:water/acetonitrile with 0.1% TFA) to afford 4-((7-methoxyquinolin-4-yl)oxy)-N-methylbenzenesulfonimidamide. 1.0 g LC-MS: m/z [M+H]⁺ 344.1.

### Step 4: Preparation of (R)-4-((7-methoxyquinolin-4-yl)oxy)-N-methylbenzenesulfonimidamide and (S)-4-((7-methoxyquinolin-4-yl)oxy)-N-methylbenzenesulfonimidamide

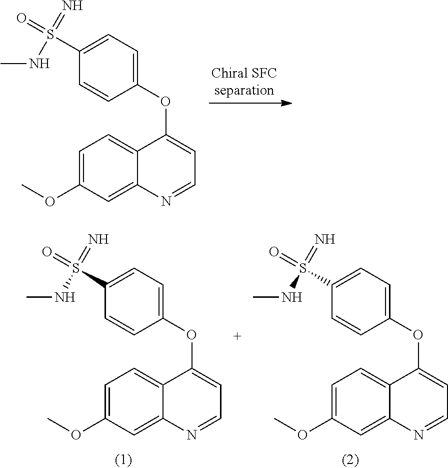

4-((7-methoxyquinolin-4-yl)oxy)-N-methylbenzenesulfonimidamide (1 g) was separated by chiral SFC chromatography (Chiralcel OD, Mobile phase: CO₂ and 45% methanol w/0.1% NH₃H₂O) to afford (R)-4-((7-methoxyquinolin-4-yl)oxy)-N-methylbenzenesulfonimidamide (400 mg) & (S)-4-((7-methoxyquinolin-4-yl)oxy)-N-methylbenzenesulfonimidamide (316 mg).

Peak 1: LC-MS: m/z [M+H]⁺ 344.1; ¹H NMR: (CD₃OD, 400 MHz) δ=8.62 (d, J=5.29 Hz, 1H), 8.23 (d, J=9.26 Hz, 1H), 8.07 (d, J=8.82 Hz, 2H), 7.46-7.37 (m, 3H), 7.31 (dd, J=9.15, 2.32 Hz, 1H), 6.68 (d, J=5.51 Hz, 1H), 3.99 (s, 3H), 2.58 (s, 3H).

Peak 2: LC-MS: m/z [M+H]⁺ 344.1: ¹H NMR: (CD₃OD, 400 MHz) δ=8.62 (d, J=5.29 Hz, 1H), 8.23 (d, J=9.26 Hz, 1H), 8.08-8.05 (m, 2H), 7.42-7.39 (m, 3H), 7.31 (dd, J=9.26, 2.43 Hz, 1H), 6.68 (d, J=5.29 Hz, 1H), 3.99 (s, 3H), 2.58 (s, 3H).

### Example 3. General Scheme—Synthesis of Compounds (15 and 16)

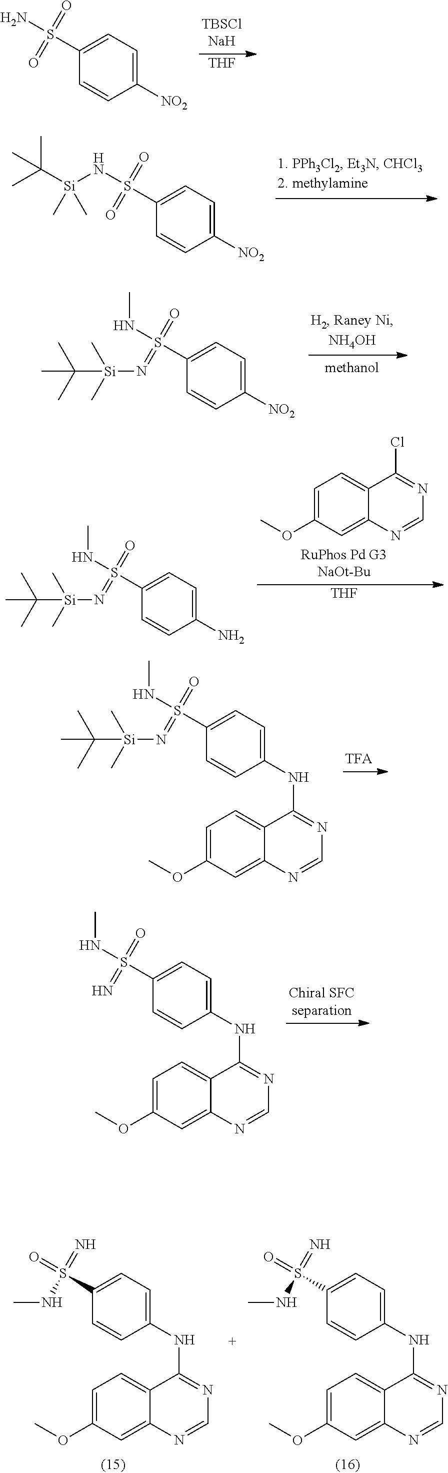

### Example 4: Exemplary Scheme—Synthesis of Compound (15 and 16)

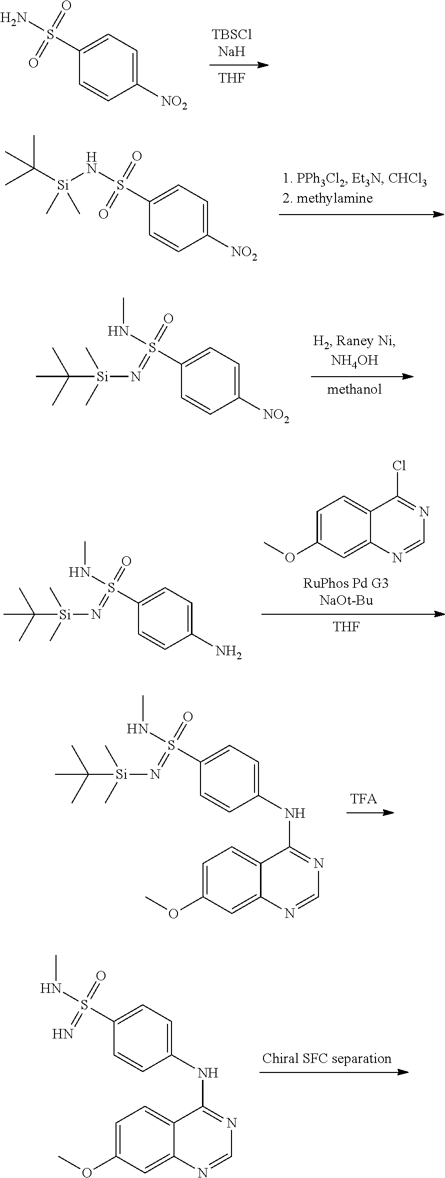

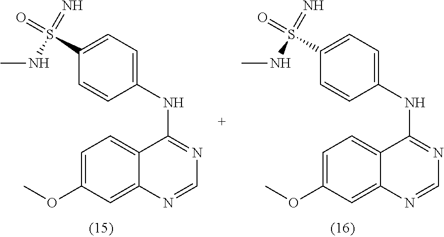 [SMILES: CN[S@@](=N)(=O)c1ccc(Nc2ncnc3cc(OC)ccc23)cc1 ; CN[S@](=N)(=O)c1ccc(Nc2ncnc3cc(OC)ccc23)cc1]

### Step 1: Preparation of N-(tert-butyldimethylsilyl)-4-nitrobenzenesulfonamide

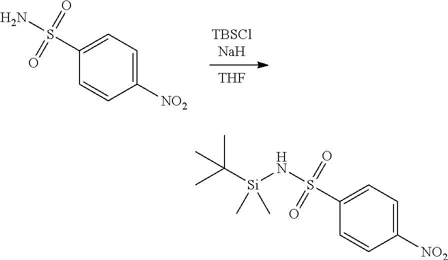 [SMILES: NS(=O)(=O)c1ccc([N+](=O)[O-])cc1 ; CC(C)(C)[Si](C)(C)NS(=O)(=O)c1ccc([N+](=O)[O-])cc1 ; "THF"]

To a solution of sodium hydride (1.48 g, 37.1 mmol, 60% dispersion in oil) in tetrahydrofuran (20 mL) was added dropwise the solution of 4-nitrobenzenesulfonamide (3.0 g, 14.84 mmol) in tetrahydrofuran (50 mL) at 5° C. over 2 minutes. After stirring for 30 min, t-butyldimethylchlorosilane (2.68 g, 17.8 mmol) in tetrahydrofuran (10 mL) was added dropwise at 5° C. The resulting mixture was stirred at 20° C. for 12 h. The reaction mixture was concentrated under reduced pressure and the resulting crude mixture was diluted with water (80 mL) and extracted with ethyl acetate (2×80 mL). The combined organic layers was washed with water (2×100 mL), dried over Na₂SO₄, filtered, and concentrated under reduced pressure. The crude product was stirred with petroleum ether (20 mL) for 10 minutes, filtered, and dried under vacuum to give the title compound. 4.6 g, LC-MS: m/z [M+H]⁺ 317.1

### Step 2: Preparation of N′-(tert-butyldimethylsilyl)-N-methyl-4-nitrobenzenesulfonimidamide

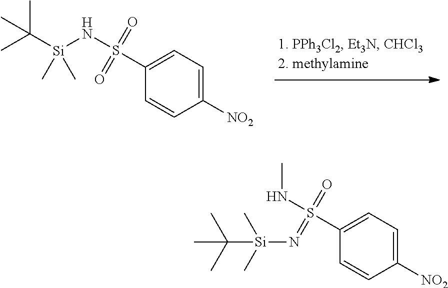 [SMILES: CC(C)(C)[Si](C)(C)NS(=O)(=O)c1ccc([N+](=O)[O-])cc1 ; CNS(=O)(=N[Si](C)(C)C(C)(C)C)c1ccc([N+](=O)[O-])cc1]

To a solution of N-(tert-butyldimethylsilyl)-4-nitrobenzenesulfonamide (429 mg, 1.36 mmol) in chloroform (15 mL) was added triethylamine (412 mg, 4.06 mmol). After stirring the mixture at 20° C. for 10 minutes, the mixture was cooled to 0° C., and dichlorotriphenylphosphorane (496 mg, 1.49 mmol) was added. After stirring at 0° C. for 30 minutes, 30% methylamine (185 mg, 3.58 mmol) was added and the mixture was stirred at 0° C. for 30 minutes. The reaction was repeated 3 times and combined. The resulting mixture was washed with water, organic layer separated, dried with Na₂SO₄, filtered, and concentrated. The product was purified by silica chromatography using ethyl acetate in petroleum ether to give the title compound. 600 mg, LC-MS: m/z [M+H]⁺ 330.1.

### Step 3: Preparation of 4-amino-N′-(tert-butyldimethylsilyl)-N-methylbenzenesulfonimidamide

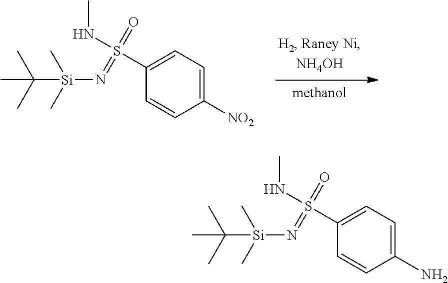 [SMILES: CNS(=O)(=N[Si](C)(C)C(C)(C)C)c1ccc([N+](=O)[O-])cc1 ; CNS(=O)(=N[Si](C)(C)C(C)(C)C)c1ccc(N)cc1]

To a solution of N′-(tert-butyldimethylsilyl)-N-methyl-4-nitrobenzenesulfonimidamide (200 mg, 0.607 mmol) in methanol (2 mL) was added concentrated ammonium hydroxide (4.2 uL) and Raney-Ni (133 mg, 1.56 mmol) under nitrogen gas. The reaction vessel was purged with hydrogen gas and stirred for 30 minutes under hydrogen at ambient temperature. The reaction mixture was filtered and concentrated under reduced pressure to give the title compound which was taken to the next step without further purification. 250 mg, LC-MS: m/z [M+H]⁺ 300.1.

### Step 4: Preparation of N′-(tert-butyldimethylsilyl)-4-((7-methoxyquinazolin-4-yl)amino)-N-methylbenzenesulfonimidamide

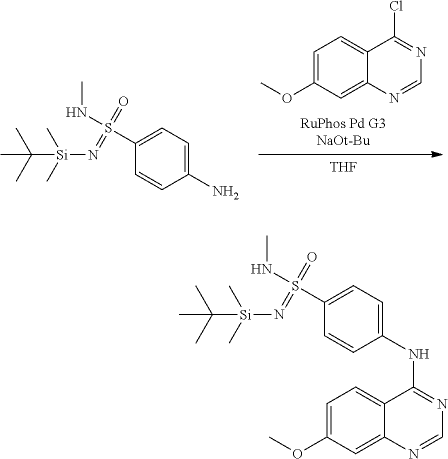 [SMILES: CNS(=O)(=N[Si](C)(C)C(C)(C)C)c1ccc(N)cc1 ; COc1ccc2c(Cl)ncnc2c1 ; CNS(=O)(=N[Si](C)(C)C(C)(C)C)c1ccc(Nc2ncnc3cc(OC)ccc23)cc1 ; "THF"]

A mixture of 4-amino-N′-(tert-butyldimethylsilyl)-N-methylbenzenesulfonimidamide (100 mg, 334 umol), 4-chloro-7-methoxyquinazoline (71 mg, 367 umol, 1.1 eq), sodium tert-butoxide (64 mg, 668 umol), and RuPhos Pd G3 (27 mg, 33.4 umol) in THF (3 mL) was degassed and purged with nitrogen. The mixture was stirred at 72° C. for 12 hours under nitrogen gas. The reaction mixture was filtered and concentrated under vacuum and title compound was taken to the next step without further purification.

### Step 5: Preparation of N′-(tert-butyldimethylsilyl)-4-((7-methoxyquinazolin-4-yl)amino)-N-methylbenzenesulfonimidamide

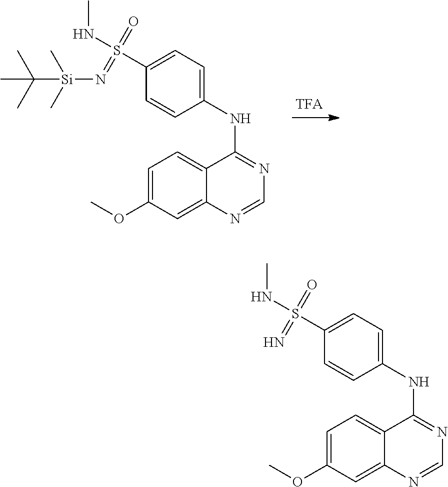 [SMILES: CNS(=O)(=N[Si](C)(C)C(C)(C)C)c1ccc(Nc2ncnc3cc(OC)ccc23)cc1 ; CNS(=N)(=O)c1ccc(Nc2ncnc3cc(OC)ccc23)cc1]

The crude mixture containing N′-(tert-butyldimethylsilyl)-4-((7-methoxyquinazolin-4-yl)amino)-N-methylbenzenesulfonimidamide in DCM (10 mL) was added TFA (373 mg, 3.3 mmol). The mixture was stirred at 20° C. for 30 minutes. The reaction mixture was filtered and concentrated under reduced pressure. The product was purified by reverse phase HPLC purification (eluent:water/acetonitrile) to give the title compound.

### Step 6: Preparation of (R)-4-((7-methoxyquinazolin-4-yl)amino)-N-methylbenzenesulfonimidamide and (S)-4-((7-methoxyquinazolin-4-yl)amino)-N-methylbenzenesulfonimidamide

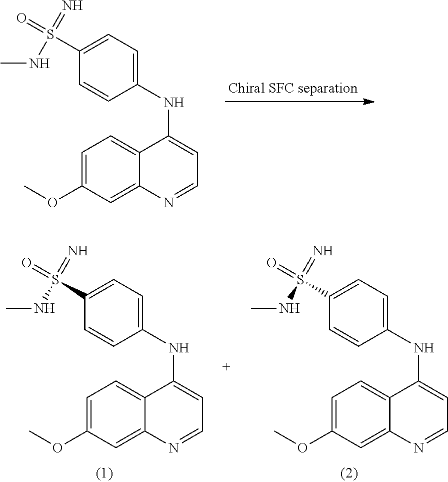 [SMILES: CNS(=N)(=O)c1ccc(Nc2ccnc3cc(OC)ccc23)cc1 ; CN[S@@](=N)(=O)c1ccc(Nc2ccnc3cc(OC)ccc23)cc1 ; CN[S@](=N)(=O)c1ccc(Nc2ccnc3cc(OC)ccc23)cc1]

N′-(tert-butyldimethylsilyl)-4-((7-methoxyquinazolin-4-yl)amino)-N-methylbenzenesulfonimidamide was separated by chiral SFC chromatography (REGIS (S, S) WHELK-01, Mobile phase: CO₂ and 55% isopropanol w/0.1% NH₃H₂O) to afford (R)-4-((7-methoxyquinazolin-4-yl)amino)-N-methylbenzenesulfonimidamide (19 mg) and (S)-4-((7-methoxyquinazolin-4-yl)amino)-N-methylbenzenesulfonimidamide (19 mg).

Peak 1: LC-MS: m/z [M+H]⁺ 344.1: ¹H NMR: (400 MHz, DMSO-d6) δ=8.63 (s, 1H), 8.49 (d, J=9.3 Hz, 1H), 8.04 (d, J=8.8 Hz, 2H), 7.85 (d, J=8.8 Hz, 2H), 7.30 (dd, J=2.6, 9.3 Hz, 1H), 7.21 (d, J=2.4 Hz, 1H), 3.92 (s, 3H), 2.83 (s, 3H).

Peak 2: LC-MS: m/z [M+H]⁺ 344.1: ¹H NMR: (400 MHz, DMSO-d6) δ=8.59 (s, 1H), 8.48 (d, J=9.0 Hz, 1H), 8.05 (d, J=8.8 Hz, 2H), 7.83 (d, J=8.8 Hz, 2H), 7.29 (dd, J=2.5, 9.2 Hz, 1H), 7.21 (d, J=2.4 Hz, 1H), 3.92 (s, 3H), 2.36 (s, 3H).

### Example 5. Exemplary Scheme—Synthesis of Compound (22)

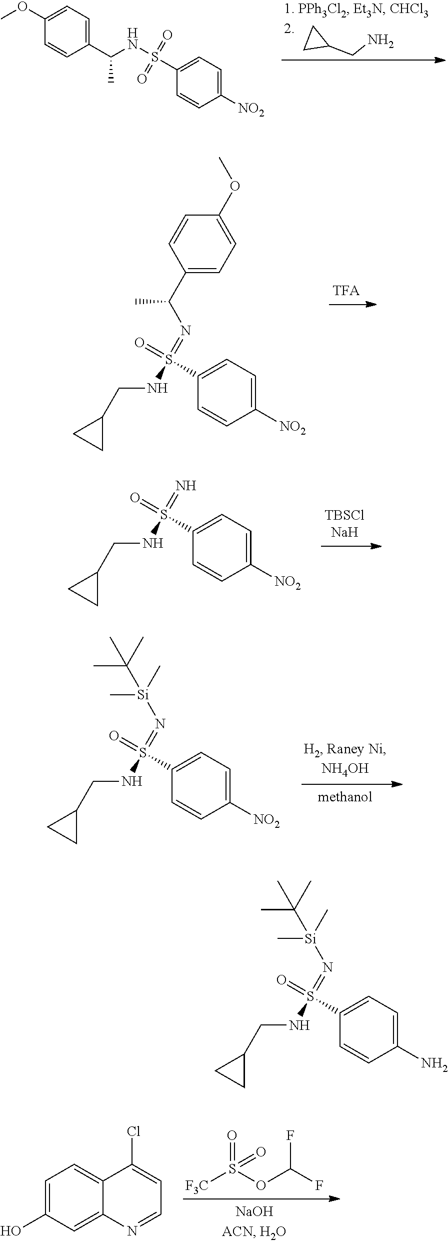

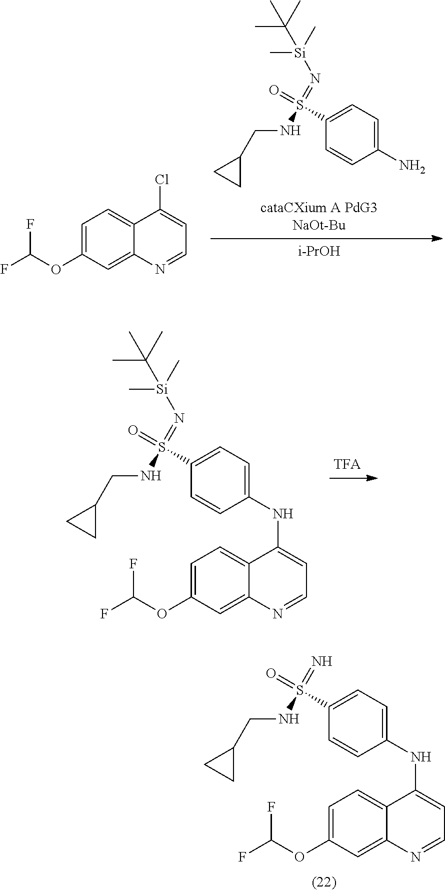 [SMILES: "i-PrOH" ; FC(F)Oc1ccc2c(Cl)ccnc2c1 ; CC(C)(C)[Si](C)(C)N=[S@](=O)(NCC1CC1)c1ccc(N)cc1 ; CC(C)(C)[Si](C)(C)N=[S@](=O)(NCC1CC1)c1ccc(Nc2ccnc3cc(OC(F)F)ccc23)cc1 ; N=[S@](=O)(NCC1CC1)c1ccc(Nc2ccnc3cc(OC(F)F)ccc23)cc1]

### Step 1: Preparation of (S)—N-(cyclopropylmethyl)-N′—((R)-1-(4-methoxyphenyl)ethyl)-4-nitrobenzenesulfonimidamide

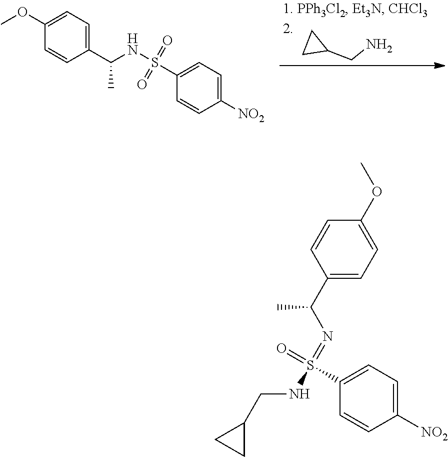 [SMILES: COc1ccc([C@@H](C)NS(=O)(=O)c2ccc([N+](=O)[O-])cc2)cc1 ; NCC1CC1 ; COc1ccc([C@@H](C)N=[S@](=O)(NCC2CC2)c2ccc([N+](=O)[O-])cc2)cc1]

The title compound was synthesized using a similar procedure described in Example 4, step 2 using cyclopropylmethanamine in place of methylamine. The diastereomers generated from the reaction were separated by silica gel chromatography using ethyl acetate in hexanes. LC-MS: m/z [M+H]⁺ 390.4

### Step 2: Preparation of (S)—N-(cyclopropylmethyl)-4-nitrobenzenesulfonimidamide

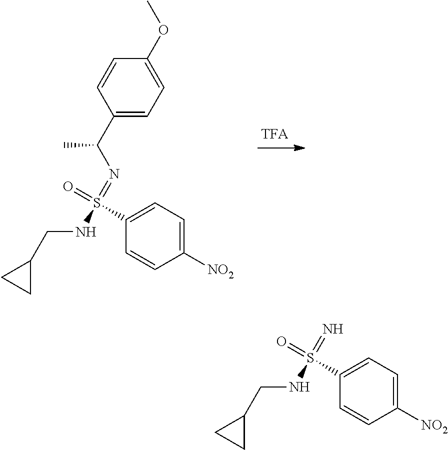 [SMILES: COc1ccc([C@@H](C)N=[S@](=O)(NCC2CC2)c2ccc([N+](=O)[O-])cc2)cc1 ; N=[S@](=O)(NCC1CC1)c1ccc([N+](=O)[O-])cc1]

The title compound was synthesized using a similar procedure described in Example 7, step 4. The product was purified by silica gel chromatography using ethyl acetate in hexanes. LC-MS: m/z [M+H]⁺ 256.1

### Step 3: Preparation of (R)—N′-(tert-butyldimethylsilyl)-N-(cyclopropylmethyl)-4-nitrobenzenesulfonimidamide

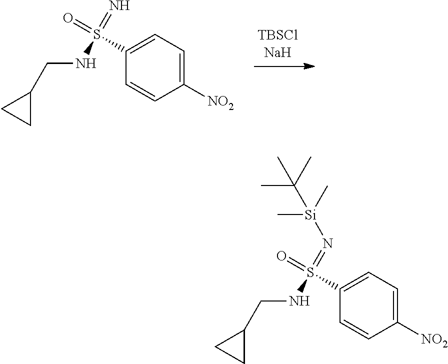 [SMILES: N=[S@](=O)(NCC1CC1)c1ccc([N+](=O)[O-])cc1 ; CC(C)(C)[Si](C)(C)N=[S@](=O)(NCC1CC1)c1ccc([N+](=O)[O-])cc1]

The title compound was synthesized using a similar procedure described in Example 4, step 1 using(S)—N-(cyclopropylmethyl)-4-nitrobenzenesulfonimidamide in place of 4-nitrobenzenesulfonamide. The product was taken to the next step without further purification. LC-MS: m/z [M−H]⁻ 368.1

### Step 4: Preparation of (R)-4-amino-N′-(tert-butyldimethylsilyl)-N-(cyclopropylmethyl)benzenesulfonimidamide

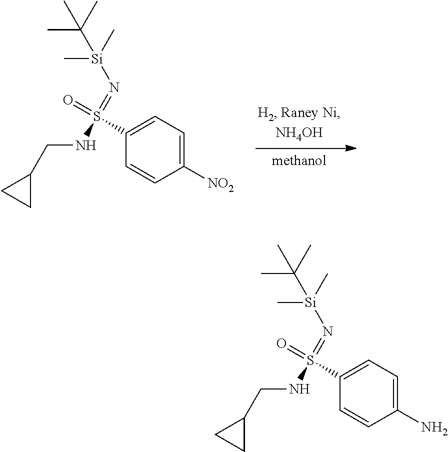 [SMILES: CC(C)(C)[Si](C)(C)N=[S@](=O)(NCC1CC1)c1ccc([N+](=O)[O-])cc1 ; CC(C)(C)[Si](C)(C)N=[S@](=O)(NCC1CC1)c1ccc(N)cc1]

The title compound was synthesized using a similar procedure described in Example 4, step 3 using (R)—N′-(tert-butyldimethylsilyl)-N-(cyclopropylmethyl)-4-nitrobenzenesulfonimidamide in place of N′-(tert-butyldimethylsilyl)-N-methyl-4-nitrobenzenesulfonimidamide. The product was purified by silica gel chromatography using ethyl acetate in hexanes. LC-MS: m/z [M−H]⁻ 338.2

### Step 5: Preparation of 4-chloro-7-(difluoromethoxy)quinoline

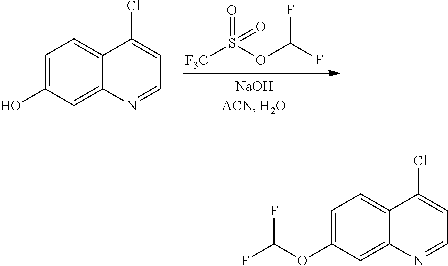

To a solution of 4-chloroquinolin-7-ol (200 mg, 1.1 mmol) in acetonitrile (5 mL) was added difluoromethyl trifluoromethanesulfonate (446 mg, 2.2 mmol) followed by a solution of 6M NaOH (220 mg, 5.5 mmol). The mixture was stirred at 20° C. for 1 hr. The reaction mixture was poured into water (100 mL) and ethyl acetate (100 mL) and the organic layer was concentrated under vacuum. The product was purified by silica chromatography (petroleum ether:ethyl acetate 2:1) to give the title compound (100 mg). LC-MS: m/z [M+H]⁺ 229.9

### Step 6: Preparation of (R)—N′-(tert-butyldimethylsilyl)-N-(cyclopropylmethyl)-4-((7-(difluoromethoxy)quinolin-4-yl)amino)benzenesulfonimidamide

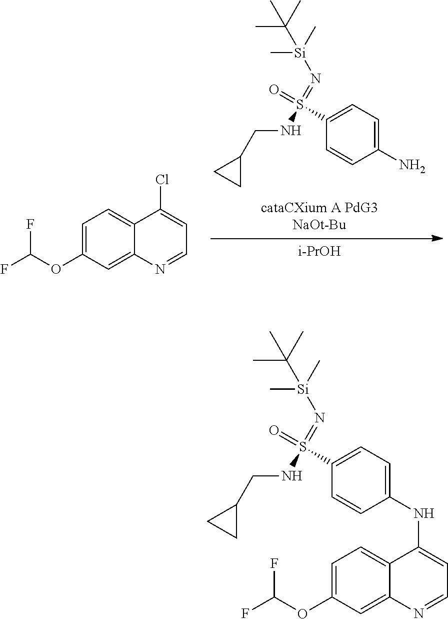 [SMILES: "i-PrOH" ; FC(F)Oc1ccc2c(Cl)ccnc2c1 ; CC(C)(C)[Si](C)(C)N=[S@](=O)(NCC1CC1)c1ccc(N)cc1 ; CC(C)(C)[Si](C)(C)N=[S@](=O)(NCC1CC1)c1ccc(Nc2ccnc3cc(OC(F)F)ccc23)cc1]

To a solution of 4-chloro-7-(difluoromethoxy)quinoline (60 mg, 261 umol) and 4-[S-[[tert-butyl (dimethyl) silyl]amino]-N-(cyclopropylmethyl) sulfonimidoyl]aniline (89 mg, 261 umol) in i-PrOH (5 mL) was added t-BuONa (38 mg, 391 umol) and cataCXium A Pd-G3 (22 mg, 26 umol). The reaction mixture was purged with inert gas and stirred at 100° C. for 12 hours. The reaction mixture was filtered and concentrated under reduced pressure to give a crude product. The crude product was taken to the next step without further purification.

### Step 7: Preparation of (S)—N-(cyclopropylmethyl)-4-((7-(difluoromethoxy)quinolin-4-yl)amino)benzenesulfonimidamide (22)

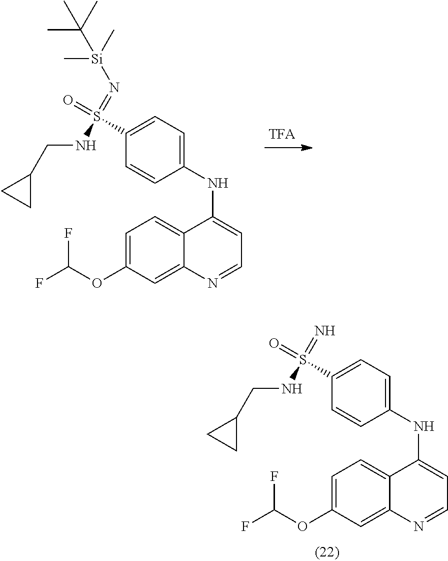 [SMILES: CC(C)(C)[Si](C)(C)N=[S@](=O)(NCC1CC1)c1ccc(Nc2ccnc3cc(OC(F)F)ccc23)cc1 ; N=[S@](=O)(NCC1CC1)c1ccc(Nc2ccnc3cc(OC(F)F)ccc23)cc1]

A crude mixture containing (R)—N′-(tert-butyldimethylsilyl)-N-(cyclopropylmethyl)-4-((7-(difluoromethoxy)quinolin-4-yl)amino)benzenesulfonimidamide was dissolved in DCM (5 mL) and TFA (107 mg, 939 umol). After stirring at 20° C. for 1 hour, the reaction mixture was concentrated and the product was purified by reverse phase HPLC purification (eluent:water/acetonitrile) to give the title compound. LC-MS: m/z [M+H]⁺ 419.1; (DMSO-d6, 400 MHz) δ=8.49 (d, J=5.3 Hz, 1H), 8.34 (d, J=9.3 Hz, 1H), 7.78 (d, J=8.7 Hz, 2H), 7.55-7.50 (m, 1H), 7.37-7.33 (m, 3H), 7.36 (t, 1H), 7.10 (d, J=5.3 Hz, 1H), 2.55 (d, J=6.4 Hz, 2H), 0.73 (br s, 1H), 0.27-0.25 (m, 2H), 0.01-0.00 (m, 2H)

### Example 6. General Scheme—Synthesis of Compound (27 and 28)

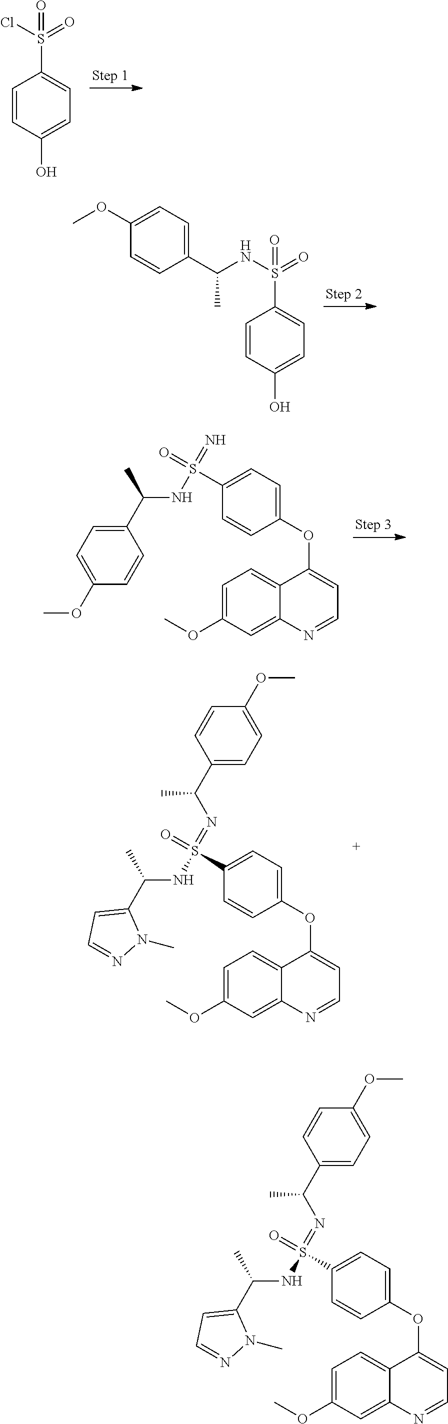

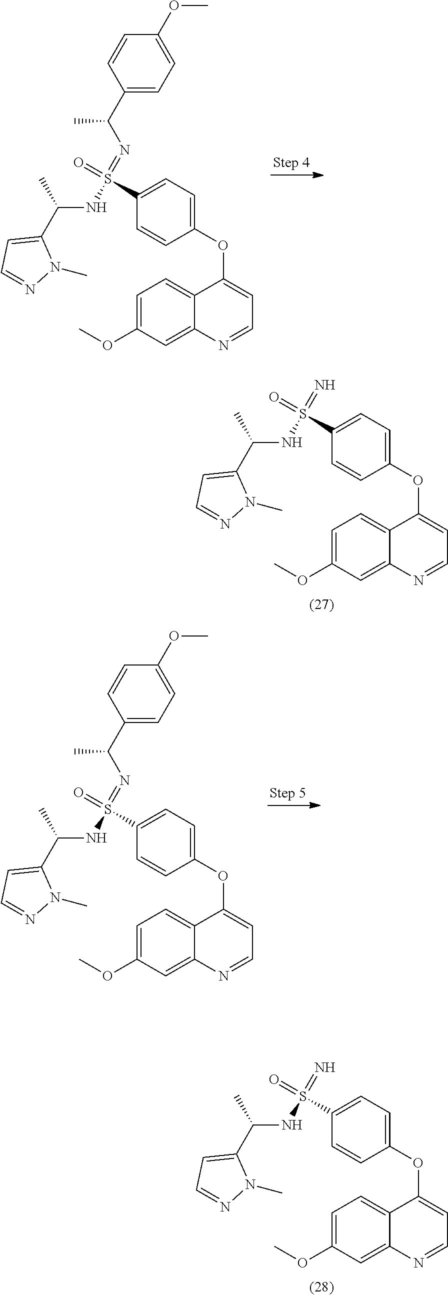 [SMILES: COc1ccc([C@@H](C)N=[S@@](=O)(N[C@@H](C)c2ccnn2C)c2ccc(Oc3ccnc4cc(OC)ccc34)cc2)cc1 ; COc1ccc2c(Oc3ccc([S@](=N)(=O)N[C@@H](C)c4ccnn4C)cc3)ccnc2c1 ; COc1ccc([C@@H](C)N=[S@](=O)(N[C@@H](C)c2ccnn2C)c2ccc(Oc3ccnc4cc(OC)ccc34)cc2)cc1 ; COc1ccc2c(Oc3ccc([S@@](=N)(=O)N[C@@H](C)c4ccnn4C)cc3)ccnc2c1]

### Example 7. Exemplary Scheme—Synthesis of Compounds (27 and 28)

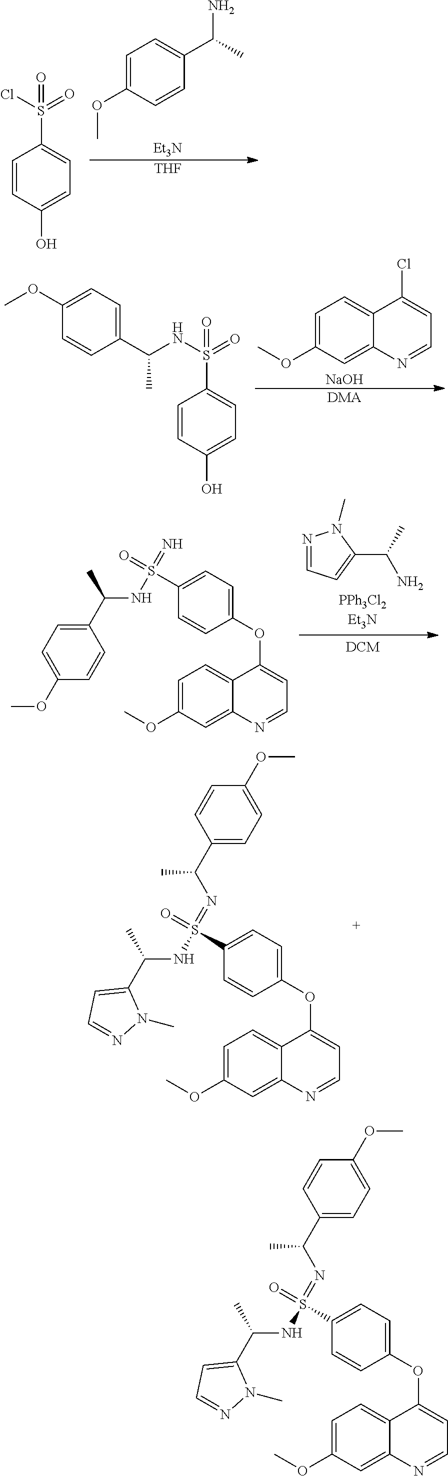

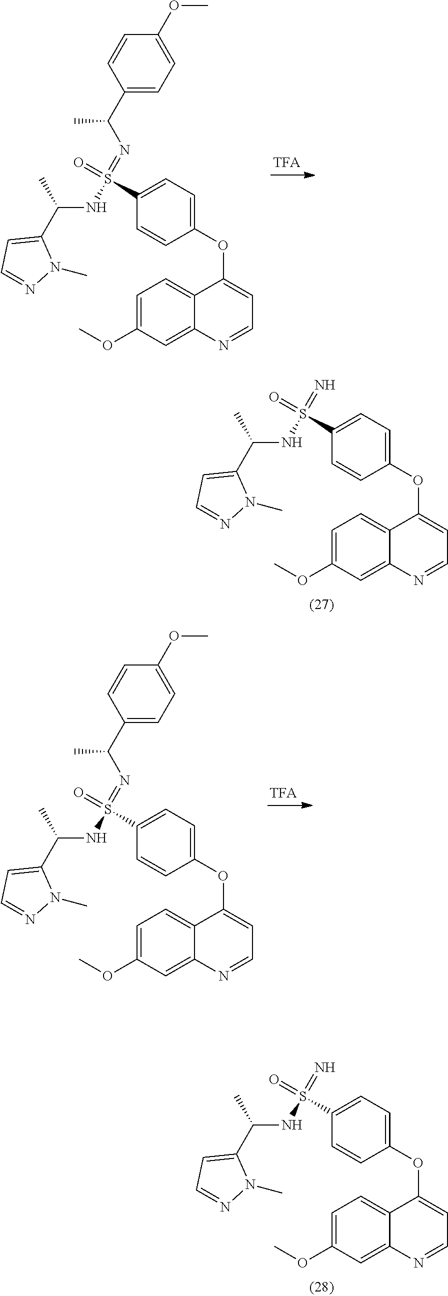 [SMILES: COc1ccc([C@@H](C)N=[S@@](=O)(N[C@@H](C)c2ccnn2C)c2ccc(Oc3ccnc4cc(OC)ccc34)cc2)cc1 ; COc1ccc2c(Oc3ccc([S@](=N)(=O)N[C@@H](C)c4ccnn4C)cc3)ccnc2c1 ; COc1ccc([C@@H](C)N=[S@](=O)(N[C@@H](C)c2ccnn2C)c2ccc(Oc3ccnc4cc(OC)ccc34)cc2)cc1 ; COc1ccc2c(Oc3ccc([S@@](=N)(=O)N[C@@H](C)c4ccnn4C)cc3)ccnc2c1]

### Step 1: Preparation of (R)-4-hydroxy-N-(1-(4-methoxyphenyl)ethyl)benzenesulfonamide

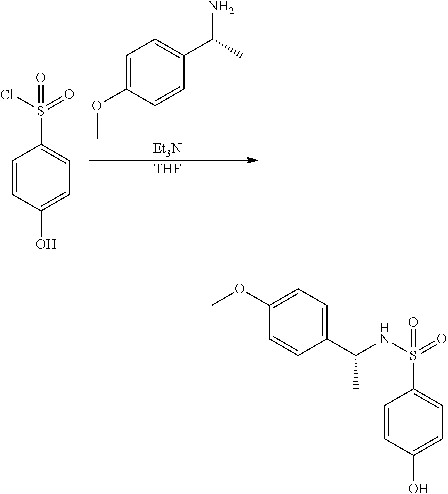

To a solution of (R)-1-(4-methoxyphenyl)ethan-1-amine (8 g, 52.9 mmol) in THF (50 mL) was added triethylamine (9.2 g, 66.1 mmol) followed by 4-hydroxybenzenesulfonyl chloride (10.2 g, 52.9 mmol) at 0° C. After stirring for 3 h, the mixture was diluted with water and extracted with EtOAc (3×20 mL). The combined organic layers were washed with brine (50 mL), dried over anhydrous sodium sulfate, filtered, and concentrated under reduced pressure to give a residue. The residue was purified by silica chromatography to give the title compound. 12.4 g LC-MS: m/z [M+H]⁺ 307.4; (400 MHz, methanol-d4) δ=7.48 (d, 2H), 7.04-6.99 (m, 2H), 6.76-6.68 (m, 4H), 4.30 (q, 1H), 3.73 (s, 3H), 1.31 (d, 3H)

### Step 2: (R)—N-(1-(4-methoxyphenyl)ethyl)-4-((7-methoxyquinolin-4-yl)oxy)benzenesulfonamide

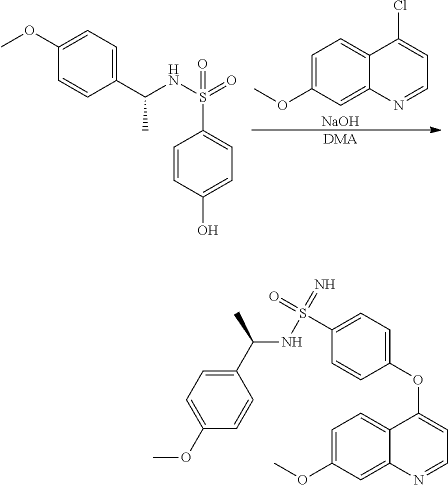

To a solution of (R)-4-hydroxy-N-(1-(4-methoxyphenyl)ethyl)benzenesulfonamide (8.0 g, 26.0 mmol) in methanol (50 ml) was added 10N sodium hydroxide (2.2 mL) and the resulting mixture was concentrated and dried under vacuum. The resulting solid and 4-chloro-7-methoxyquinoline (4.2 g, 21.7 mmol) were dissolved with N,N-dimethylacetamide (21 mL) and the mixture was heated at 140° C. for 10 h. After cooling to room temperature, the mixture was diluted with EtOAc (100 ml) and washed with 2 portions of 5% LiCl in water (100 mL). The organic layer was dried with anhydrous sodium sulfate, filtered, and concentrated in vacuo. The product was purified by silica gel chromatography using ethyl acetate in hexanes (0-100%). LC-MS: m/z [M+H]⁺ 465.6

### Step 3: (R)—N′—((R)-1-(4-methoxyphenyl)ethyl)-4-((7-methoxyquinolin-4-yl)oxy)-N—((S)-1-(1-methyl-1H-pyrazol-5-yl)ethyl)benzenesulfonimidamide and (S)—N′—((R)-1-(4-methoxyphenyl)ethyl)-4-((7-methoxyquinolin-4-yl)oxy)-N—((S)-1-(1-methyl-1H-pyrazol-5-yl)ethyl)benzenesulfonimidamide

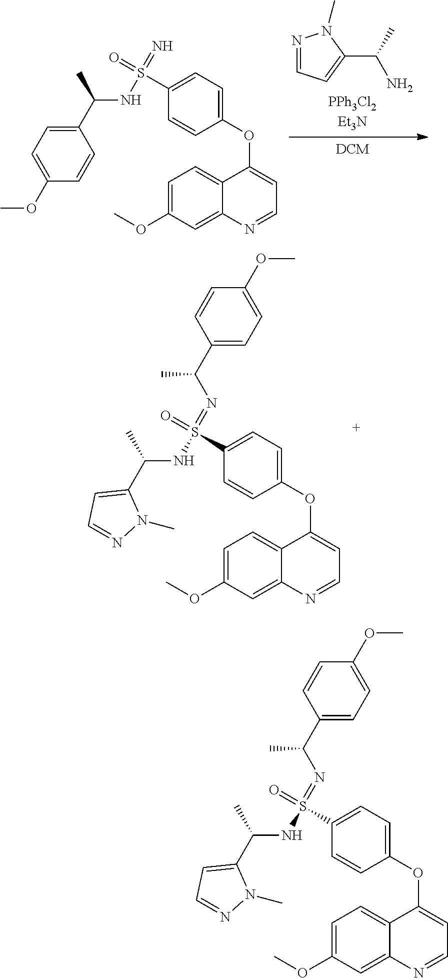

The title compound was synthesized using a similar procedure described in Example 4, step 2 using(S)-1-(1-methyl-1H-pyrazol-5-yl)ethan-1-amine in place of methylamine. The products were purified by reverse phase HPLC purification (eluent:water/acetonitrile w/0.1% TFA) to give the title compound. Peak 1: LC-MS: m/z [M+H]⁺ 572.3; Peak 2: LC-MS: m/z [M+H]⁺ 572.1

### Step 4: ((R)-4-((7-methoxyquinolin-4-yl)oxy)-N—((S)-1-(1-methyl-1H-pyrazol-5-yl)ethyl)benzenesulfonimidamide (27)

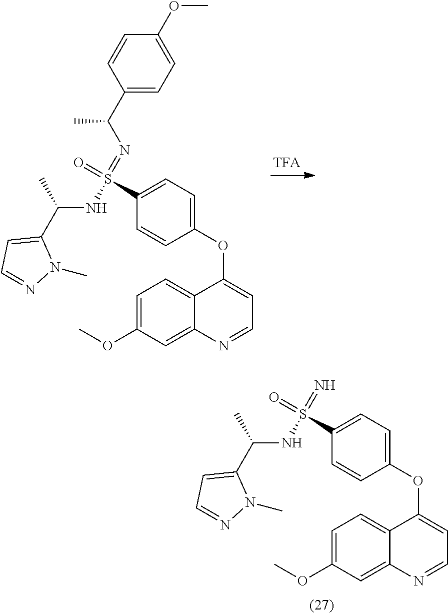 [SMILES: COc1ccc([C@@H](C)N=[S@@](=O)(N[C@@H](C)c2ccnn2C)c2ccc(Oc3ccnc4cc(OC)ccc34)cc2)cc1 ; COc1ccc2c(Oc3ccc([S@](=N)(=O)N[C@@H](C)c4ccnn4C)cc3)ccnc2c1]

A solution of (R)—N′—((R)-1-(4-methoxyphenyl)ethyl)-4-((7-methoxyquinolin-4-yl)oxy)-N—((S)-1-(1-methyl-1H-pyrazol-5-yl)ethyl)benzenesulfonimidamide in trifluoroacetic acid (0.5 mL) and DCM (0.5 mL) was stirred at 50° C. for 0.5 h. The mixture was concentrated under reduced pressure to give a residue. The product was purified by reverse phase HPLC purification (eluent: water/acetonitrile w/0.1% TFA) to give the title compound. LC-MS: m/z [M+H]⁺ 438.1

### Step 5: (S)-4-((7-methoxyquinolin-4-yl)oxy)-N—((S)-1-(1-methyl-1H-pyrazol-5-yl)ethyl)benzenesulfonimidamide (28)

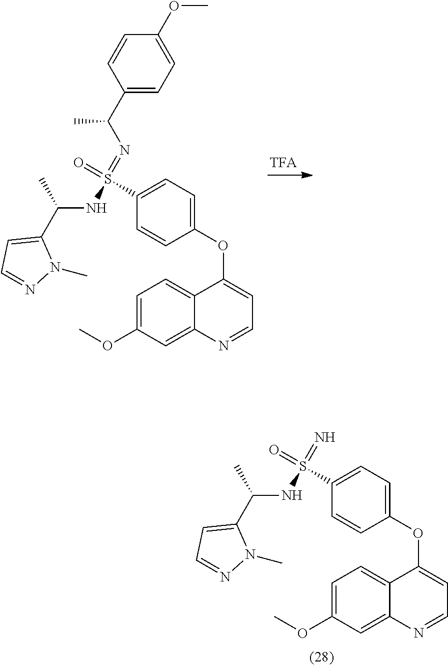 [SMILES: COc1ccc([C@@H](C)N=[S@](=O)(N[C@@H](C)c2ccnn2C)c2ccc(Oc3ccnc4cc(OC)ccc34)cc2)cc1 ; COc1ccc2c(Oc3ccc([S@@](=N)(=O)N[C@@H](C)c4ccnn4C)cc3)ccnc2c1]

A solution of (S)—N′—((R)-1-(4-methoxyphenyl)ethyl)-4-((7-methoxyquinolin-4-yl)oxy)-N—((S)-1-(1-methyl-1H-pyrazol-5-yl)ethyl)benzenesulfonimidamide in trifluoroacetic acid (0.5 mL) and DCM (0.5 mL) was stirred at 50° C. for 0.5 h. The mixture was concentrated under reduced pressure to give a residue. The product was purified by reverse phase HPLC purification (eluent: water/acetonitrile w/0.1% TFA) to give the title compound. LC-MS: m/z [M+H]⁺ 438.1

### Example 8. General Scheme—Synthesis of Compounds (56)

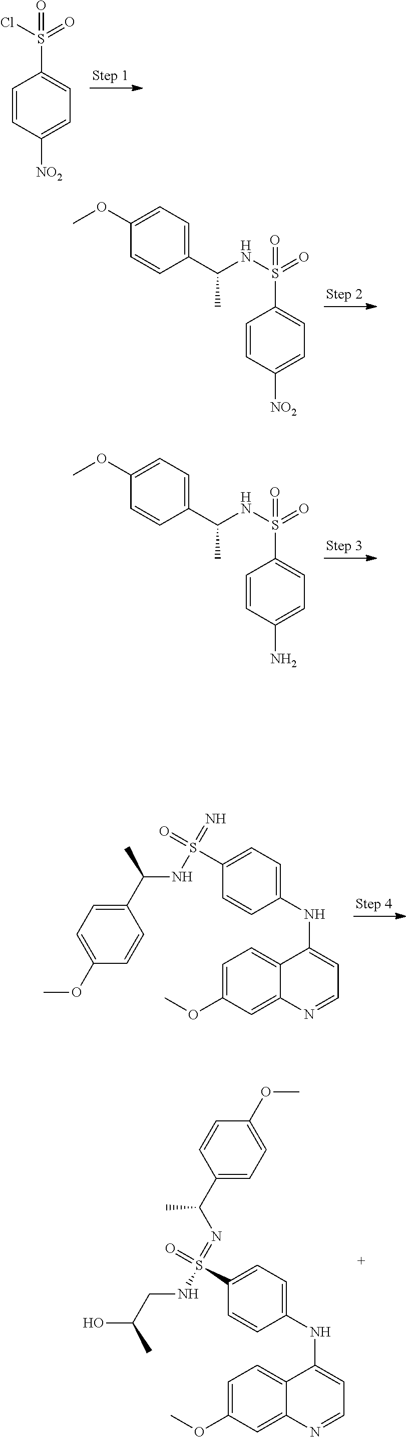 [SMILES: O=[N+]([O-])c1ccc(S(=O)(=O)Cl)cc1 ; COc1ccc([C@@H](C)NS(=O)(=O)c2ccc([N+](=O)[O-])cc2)cc1 ; COc1ccc([C@@H](C)NS(=O)(=O)c2ccc(N)cc2)cc1 ; COc1ccc([C@@H](C)NS(=N)(=O)c2ccc(Nc3ccnc4cc(OC)ccc34)cc2)cc1 ; COc1ccc([C@@H](C)N=[S@@](=O)(NC[C@@H](C)O)c2ccc(Nc3ccnc4cc(OC)ccc34)cc2)cc1]

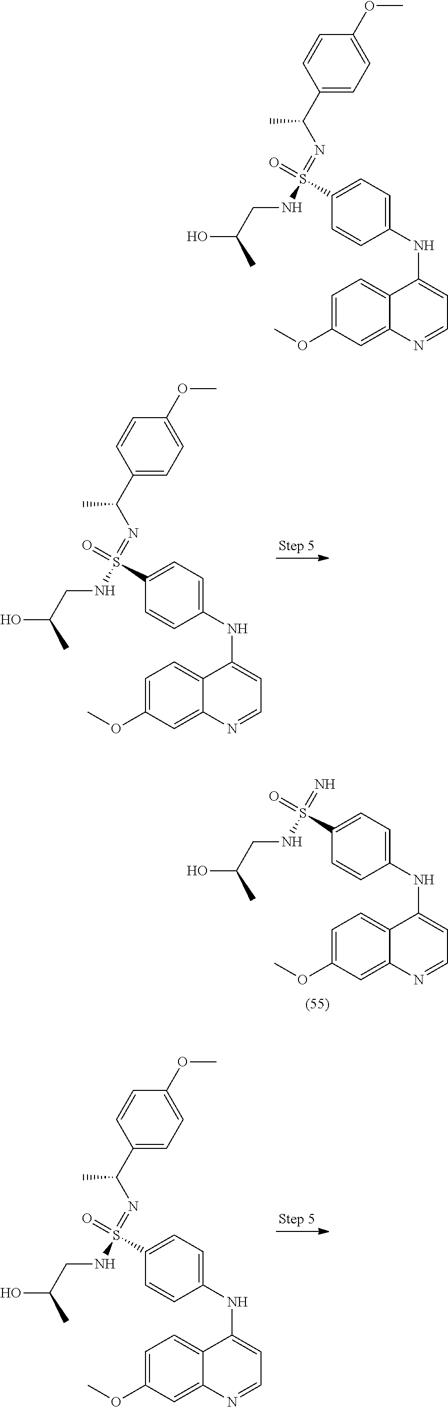 [SMILES: COc1ccc([C@@H](C)N=[S@](=O)(NC[C@@H](C)O)c2ccc(Nc3ccnc4cc(OC)ccc34)cc2)cc1 ; COc1ccc([C@@H](C)N=[S@@](=O)(NC[C@@H](C)O)c2ccc(Nc3ccnc4cc(OC)ccc34)cc2)cc1 ; COc1ccc2c(Nc3ccc([S@](=N)(=O)NC[C@@H](C)O)cc3)ccnc2c1 ; COc1ccc([C@@H](C)N=[S@](=O)(NC[C@@H](C)O)c2ccc(Nc3ccnc4cc(OC)ccc34)cc2)cc1]

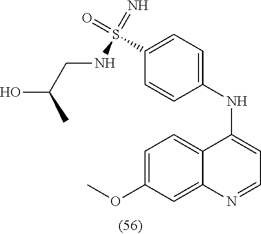 [SMILES: COc1ccc2c(Nc3ccc([S@@](=N)(=O)NC[C@@H](C)O)cc3)ccnc2c1]

### Example 9. Exemplary Scheme—Synthesis of Compound (56)

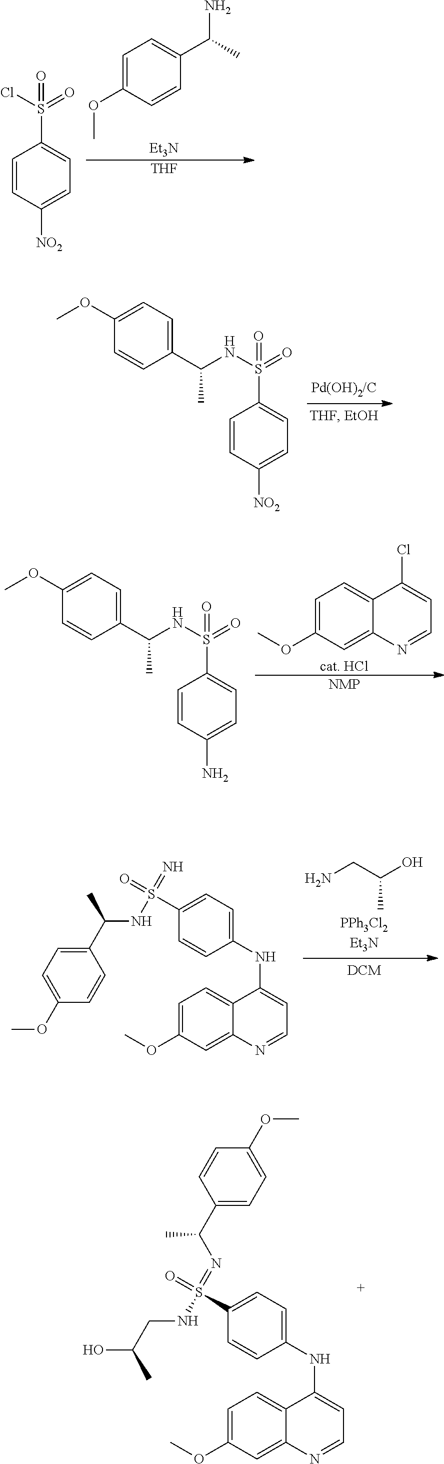

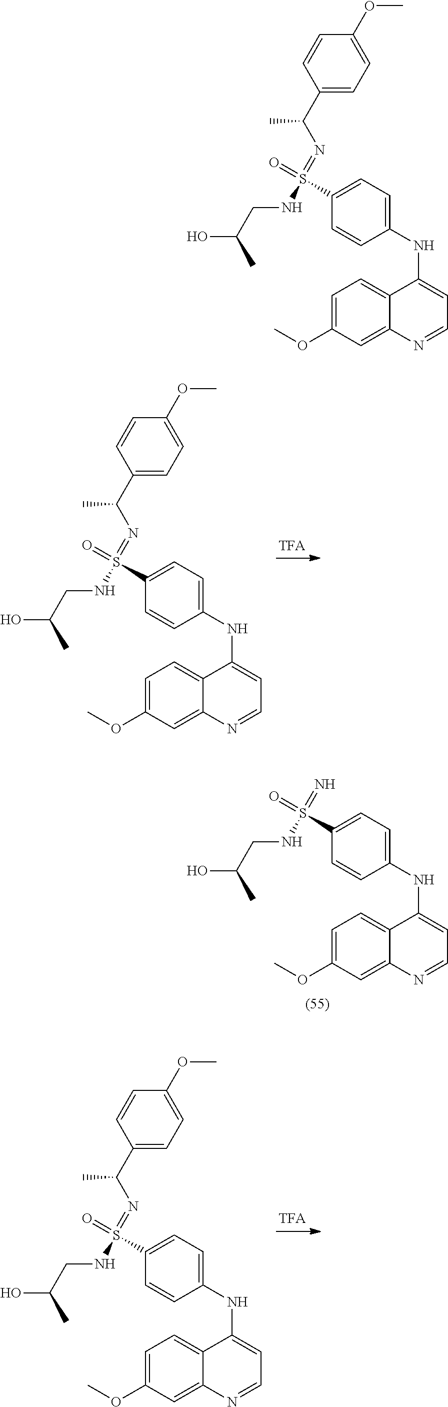 [SMILES: COc1ccc([C@@H](C)N=[S@](=O)(NC[C@@H](C)O)c2ccc(Nc3ccnc4cc(OC)ccc34)cc2)cc1 ; COc1ccc([C@@H](C)N=[S@@](=O)(NC[C@@H](C)O)c2ccc(Nc3ccnc4cc(OC)ccc34)cc2)cc1 ; COc1ccc2c(Nc3ccc([S@](=N)(=O)NC[C@@H](C)O)cc3)ccnc2c1 ; COc1ccc([C@@H](C)N=[S@](=O)(NC[C@@H](C)O)c2ccc(Nc3ccnc4cc(OC)ccc34)cc2)cc1]

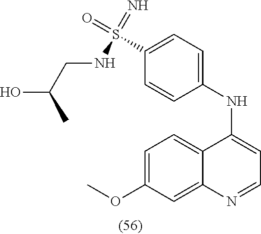 [SMILES: COc1ccc2c(Nc3ccc([S@@](=N)(=O)NC[C@@H](C)O)cc3)ccnc2c1]

### Step 1: Preparation of (R)—N-(1-(4-methoxyphenyl)ethyl)-4-nitrobenzenesulfonamide

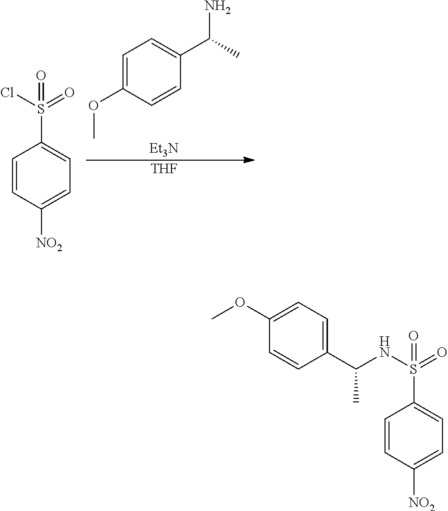

To a solution of (R)-1-(4-methoxyphenyl)ethan-1-amine (19.9 mL, 134 mmol) and triethylamine (19.6 mL, 141 mmol) in THF (120 mL) was added portionwise 4-nitrobenzenesulfonyl chloride (28.4 g, 128 mmol) at −20° C. maintaining an internal temperature below 20° C. After stirring for 30 minutes, the mixture was warmed to room temperature and 0.05N HCl (aq, 600 mL) was added to the stirring solution. The mixture was stirred for 10 minutes, solids were filtered, and washed with water (600 mL). The resulting solid was dried under vacuum at 40° C. LC-MS: m/z [M+H]⁺ 337.36.

### Step 2: Preparation of (R)-4-amino-N-(1-(4-methoxyphenyl)ethyl)benzenesulfonamide

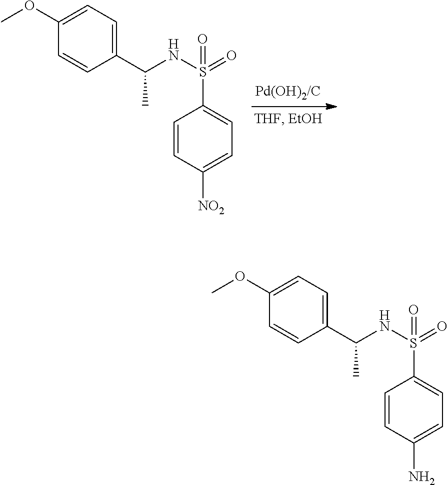 [SMILES: COc1ccc([C@@H](C)NS(=O)(=O)c2ccc([N+](=O)[O-])cc2)cc1 ; COc1ccc([C@@H](C)NS(=O)(=O)c2ccc(N)cc2)cc1]

To a flask purged with inert gas was added 20% Pd(OH)₂ on carbon (2.1 g,) followed by a solution of (R)—N-(1-(4-methoxyphenyl)ethyl)-4-nitrobenzenesulfonamide (40.4 g, 120 mmol) in EtOH (200 mL) and THF (200 mL). The reaction vessel was purged with hydrogen gas and stirred under hydrogen for 18 h. Hydrogen gas was recharged until the reaction was complete. The flask was purged with nitrogen and mixture was filtered through celite. The pad of celite was washed with THF and the filtrate was partially concentrated to ˜100 mL. To the vigorously stirring solution was added ether (200 mL) followed by hexane (300 mL). The resulting solid was dried under vacuum at 40° C. LC-MS: m/z [M+Na]⁺329.15.

### Step 3: Preparation of (R)—N-(1-(4-methoxyphenyl)ethyl)-4-((7-methoxyquinolin-4-yl)amino)benzenesulfonamide

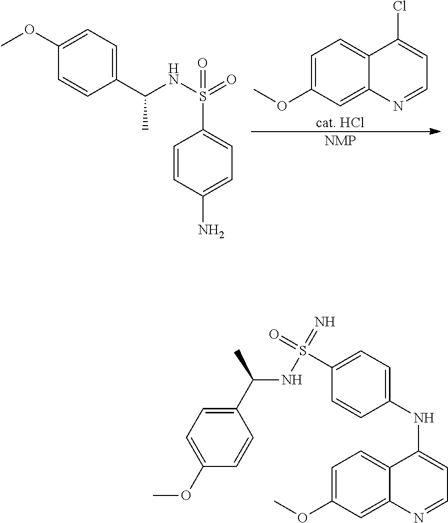

To a solution of 4-chloro-7-methoxyquinoline (9.7 g, 50.1 mmol) in NMP (48.5 mL) was added concentrated HCl (0.417 mL, 5.0 mmol). After stirring the reaction at 80° C. for 1 h, the mixture was cooled to 50° C., basified with 10N sodium hydroxide (5.6 mL), and diluted with water (400 mL). The mixture was cooled to RT and the resulting solids were filtered, washed with water (400 mL), and dried under vacuum at 40° C. to give the title compound. 21.5 g. LC-MS: m/z [M+H]⁺464.2.

### Step 4: Preparation of (R)—N—((R)-2-hydroxypropyl)-N′—((R)-1-(4-methoxyphenyl)ethyl)-4-((7-methoxyquinolin-4-yl)amino)benzenesulfonimidamide and (S)—N—((R)-2-hydroxypropyl)-N′—((R)-1-(4-methoxyphenyl)ethyl)-4-((7-methoxyquinolin-4-yl)amino)benzenesulfonimidamide

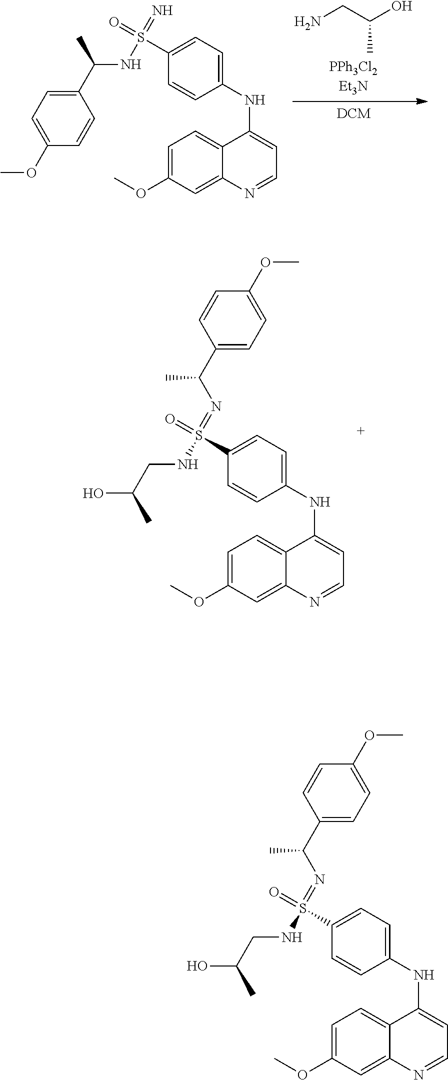

The title compound was synthesized using a similar procedure described in Example 4, step 2 using (R)-1-aminopropan-2-ol in place of methylamine. The products were purified by reverse phase HPLC purification (eluent:water/acetonitrile w/0.1% TFA) to give the title compound. Peak 1: LC-MS: m/z [M+H]⁺ 521.3; Peak 2: LC-MS: m/z [M+H]⁺ 521.3

### Step 4: Preparation of (S)—N—((R)-2-hydroxypropyl)-4-((7-methoxyquinolin-4-yl)amino)benzenesulfonimidamide (55)

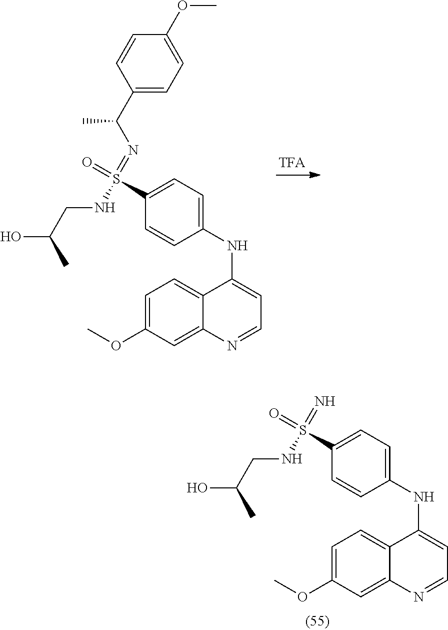 [SMILES: COc1ccc([C@@H](C)N=[S@@](=O)(NC[C@@H](C)O)c2ccc(Nc3ccnc4cc(OC)ccc34)cc2)cc1 ; COc1ccc2c(Nc3ccc([S@](=N)(=O)NC[C@@H](C)O)cc3)ccnc2c1]

The title compound was synthesized using a similar procedure described in Example 7, step 4. The products were purified by reverse phase HPLC purification (eluent:water/acetonitrile w/0.1% TFA) to give the title compound. LC-MS: m/z [M+H]⁺ 387.1; 1H NMR (400 MHZ, DMSO-d6) δ=8.63-8.47 (m, 2H), 8.09 (d, J=8.7 Hz, 2H), 7.74 (d, J=8.7 Hz, 2H), 7.48 (dd, J=2.5, 9.4 Hz, 1H), 7.40 (d, J=2.5 Hz, 1H), 7.05 (d, J=6.9 Hz, 1H), 3.98 (s, 3H), 3.68-3.64 (m, 1H), 2.87 (d, J=5.6 Hz, 2H), 1.04 (d, J=6.3 Hz, 3H)

### Step 5: Preparation of (S)—N—((R)-2-hydroxypropyl)-4-((7-methoxyquinolin-4-yl)amino)benzenesulfonimidamide (56)

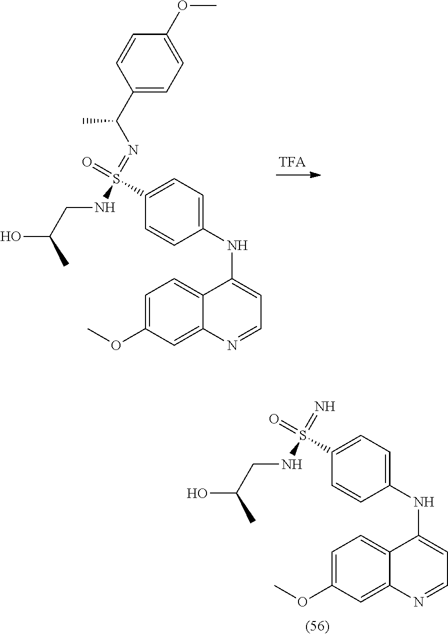 [SMILES: COc1ccc([C@@H](C)N=[S@](=O)(NC[C@@H](C)O)c2ccc(Nc3ccnc4cc(OC)ccc34)cc2)cc1 ; COc1ccc2c(Nc3ccc([S@@](=N)(=O)NC[C@@H](C)O)cc3)ccnc2c1]

The title compound was synthesized using a similar procedure described in Example 7, step 4. The products were purified by reverse phase HPLC purification (eluent:water/acetonitrile w/0.1% TFA) to give the title compound. LC-MS: m/z [M+H]⁺ 387.1; 1H NMR (400 MHZ, DMSO-d6) δ=8.63-8.52 (m, 2H), 8.10 (d, J=8.6 Hz, 2H), 7.76 (d, J=8.8 Hz, 2H), 7.47 (dd, J=2.5, 9.4 Hz, 1H), 7.42 (d, J=2.4 Hz, 1H), 7.06 (d, J=7.1 Hz, 1H), 3.97 (s, 3H), 3.72-3.62 (m, 1H), 2.86 (d, J=5.2 Hz, 2H), 1.03 (d, J=6.4 Hz, 3H)

Compounds 3-14, 25-26, and 31-34 were synthesized according to the general scheme in Example 1 using similar procedures described for compound 1 and 2.

Compounds 17-20 were synthesized according to the general scheme in Example 3 using similar procedures described for compound 15 and 16.

Compound 21 was synthesized using a similar procedure described in Example 5, using similar procedures described for compound 22.

Compound 23-24, 29-30, 43-44 and 65-66 were synthesized according to the general scheme in Example 6 using similar procedures described in compound 27 and 28.

Compound 35-42, 45-54, and 57-64 were synthesized according to the general scheme in Example 8 using similar procedures described for compound 55 and 56.

TABLE 1
Compounds 1-69

| Compound | | | LCMS: |
|---|---|---|---|
| No. | Structure-Compound Name | ¹H NMR | [M + H]⁺ |
| 1 | 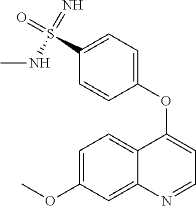 | (CD3OD, 400 MHz) δ = 8.62 (d, J = 5.29 Hz, 1H), 8.23 (d, J = 9.26 Hz, 1H), 8.08-8.05 (m, 2H), 7.42-7.39 (m, 3H), 7.31 (dd, J = 9.26, 2.43 Hz, 1H), 6.68 (d, J = 5.29 Hz, 1H), 3.99 (s, 3H), 2.58 (s, 3H) | 344.1 |
| | (R)-4-((7-methoxyquinolin-4-yl)oxy)-N- | | |
| | methylbenzenesulfonimidamide | | |
| 2 | 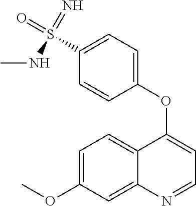 | (CD3OD, 400 MHz) δ = 8.62 (d, J = 5.29 Hz, 1H), 8.23 (d, J = 9.26 Hz, 1H), 8.07 (d, J = 8.82 Hz, 2H), 7.46-7.37 (m, 3H), 7.31 (dd, J = 9.15, 2.32 Hz, 1H), 6.68 (d, J = 5.51 Hz, 1H), 3.99 (s, 3H), 2.58 (s, 3H) | 344.1 |
| | (S)-4-((7-methoxyquinolin-4-yl)oxy)-N- | | |
| | methylbenzenesulfonimidamide | | |
| 3 | 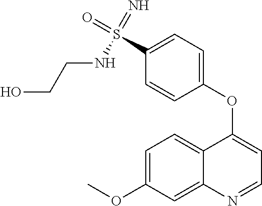 | (400 MHz, DMSO-d6) 1H NMR (400 MHz, DMSO-d6) δ = 8.66 (d, J = 5.1 Hz, 1H), 8.13 (d, J = 9.0 Hz, 1H), 7.96 (d, J = 8.7 Hz, 2H), 7.41 (d, J = 2.3 Hz, 1H), 7.37 (d, J = 8.7 Hz, 2H), 7.30 (dd, J = 2.3, 9.2 Hz, 1H), 6.64 (d, J = 5.1 Hz, 1H), 3.91 (s, 3H), 3.40-3.31 (m, 2H), 2.82-2.75 (m, 2H) | 347.1 |
| | (R)-N-(2-hydroxyethyl)-4-((7-methoxyquinolin-4- | | |
| | yl)oxy)benzenesulfonimidamide | | |
| 4 | 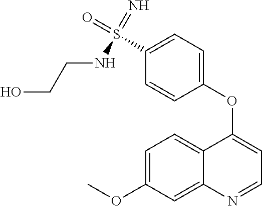 | (400 MHz, DMSO-d6) 1H NMR (400 MHz, DMSO-d6) δ = 8.64 (d, J = 5.1 Hz, 1H), 8.13 (d, J = 9.2 Hz, 1H), 7.95 (d, J = 8.7 Hz, 2H), 7.39 (d, J = 2.2 Hz, 1H), 7.36 (d, J = 8.7 Hz, 2H), 7.30 (dd, J = 2.3, 9.2 Hz, 1H), 6.64 (d, J = 5.1 Hz, 1H), 3.90 (s, 3H), 3.37-3.31 (m, 2H), 2.82-2.79 (m, 2H) | 347.1 |
| | (S)-N-(2-hydroxyethyl)-4-((7-methoxyquinolin-4- | | |
| | yl)oxy)benzenesulfonimidamide | | |
| 5 | 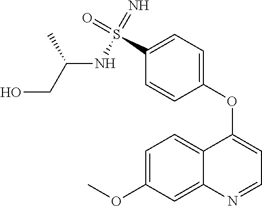 | (400 MHz, DMSO-d6) δ = 8.66 (d, J = 5.3 Hz, 1H), 8.14 (d, J = 9.0 Hz, 1H), 7.98 (d, J = 8.6 Hz, 2H), 7.42 (d, J = 2.4 Hz, 1H), 7.37 (d, J = 8.8 Hz, 2H), 7.30 (dd, J = 2.5, 9.2 Hz, 1H), 6.61 (d, J = 5.1 Hz, 1H), 3.92 (s, 3H), 3.33-3.23 (m, 1H), 3.21 − 2.99 (m, 2H), 0.80 (d, 2H) | 388.1 |
| | (R)-N-((S)-1-hydroxypropan-2-yl)-4-((7- | | |
| | methoxyquinolin-4- | | |
| | yl)oxy)benzenesulfonimidamide | | |
| 6 | 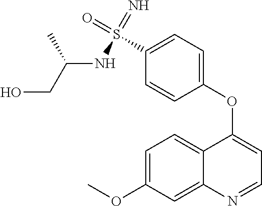 | (400 MHz, DMSO-d6) δ = 8.66 (d, J = 5.3 Hz, 1H), 8.15 (d, J = 9.0 Hz, 1H), 7.99 (d, J = 8.8 Hz, 2H), 7.41 (d, J = 2.4 Hz, 1H), 7.37 (d, J = 8.8 Hz, 2H), 7.30 (dd, J = 2.5, 9.2 Hz, 1H), 6.61 (d, J = 5.1 Hz, 1H), 3.92 (s, 3H), 3.33 − 3.20 (m, 1H), 3.16 − 3.01 (m, 2H), 0.85 (br, 3H) | 388.1 |
| | (S)-N-((S)-1-hydroxypropan-2-yl)-4-((7- | | |
| | methoxyquinolin-4- | | |
| | yl)oxy)benzenesulfonimidamide | | |
| 7 | 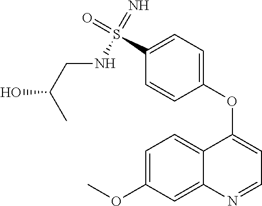 | (400 MHz, DMSO-d6) δ = 8.65 (d, J = 5.1 Hz, 1H), 8.10 (d, J = 9.0 Hz, 1H), 7.93 (d, J = 8.6 Hz, 2H), 7.39 (s, 1H), 7.36 (d, J = 8.6 Hz, 2H), 7.28 (d, J = 8.8 Hz, 1H), 6.61 (d, J = 4.9 Hz, 1H), 3.90 (s, 3H), 3.55-3.54 (m, 1H), 2.63 − 2.54 (m, 2H), 0.96 (d, J = 6.0 Hz, 3H) | 388.1 |
| | (R)-N-((S)-2-hydroxypropyl)-4-((7- | | |
| | methoxyquinolin-4- | | |
| | yl)oxy)benzenesulfonimidamide | | |
| 8 | 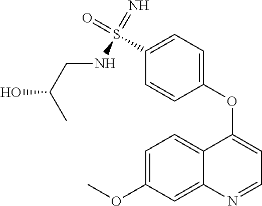 | (400 MHz, DMSO-d6) 1H NMR (400 MHz, DMSO-d6) δ = 8.67 (d, J = 5.5 Hz, 1H), 8.14 (d, J = 9.7 Hz, 1H), 7.95 (d, J = 8.6 Hz, 2H), 7.40 − 7.36 (m, 3H), 7.31-7.28 (m, 1H), 6.64 (d, J = 5.3 Hz, 1H), 3.90 (s, 3H), 3.56-3.54 (m, 1H), 2.63-2.61 (m, 2H), 0.96 (d, J = 6.2 Hz, 3H) | 388.1 |
| | (S)-N-((S)-2-hydroxypropyl)-4-((7- | | |
| | methoxyquinolin-4- | | |
| | yl)oxy)benzenesulfonimidamide | | |
| 9 | 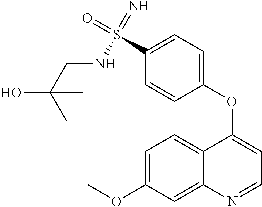 | (400 MHz, DMSO-d6) 1H NMR (400 MHz, DMSO-d6) δ = 8.67 (d, J = 5.1 Hz, 1H), 8.14 (d, J = 9.2 Hz, 1H), 7.97 (br d, J = 8.7 Hz, 2H), 7.42 (d, J = 2.1 Hz, 1H), 7.38 (d, J = 8.7 Hz, 2H), 7.31 (dd, J = 2.1, 9.1 Hz, 1H), 6.64 (d, J = 5.1 Hz, 1H), 3.93 (s, 3H), 2.64 (br s, 2H), 1.05 (s, 6H) | 402.1 |
| | (R)-N-(2-hydroxy-2-methylpropyl)-4-((7- | | |
| | methoxyquinolin-4- | | |
| | yl)oxy)benzenesulfonimidamide | | |
| 10 | 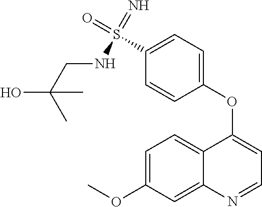 | (400 MHz, DMSO-d6) 1H NMR (400 MHz, DMSO-d6) δ = 8.68 (d, J = 5.1 Hz, 1H), 8.14 (d, J = 9.0 Hz, 1H), 7.97 (d, J = 8.8 Hz, 2H), 7.43 (d, J = 2.4 Hz, 1H), 7.39 (d, J = 8.8 Hz, 2H), 7.30 (dd, J = 2.5, 9.2 Hz, 1H), 6.64 (d, J = 5.1 Hz, 1H), 3.93 (s, 3H), 2.64 (br s, 2H), 1.05 (s, 6H) | 402.1 |
| | (S)-N-(2-hydroxy-2-methylpropyl)-4-((7- | | |
| | methoxyquinolin-4- | | |
| | yl)oxy)benzenesulfonimidamide | | |
| 11 | 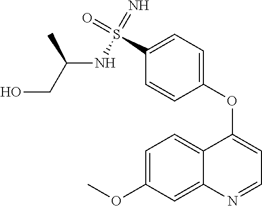 | (400 MHz, DMSO-d6) δ = 8.67 (d, J = 5.1 Hz, 1H), 8.14 (d, J = 9.3 Hz, 1H), 7.98 (d, J = 8.6 Hz, 2H), 7.42 (d, J = 2.2 Hz, 1H), 7.38 (d, J = 8.6 Hz, 2H), 7.30 (dd, J = 2.3, 9.2 Hz, 1H), 6.61 (d, J = 5.3 Hz, 1H), 3.92 (s, 3H), 3.28 (dd, J = 4.9, 10.1 Hz, 1H), 3.08 − 3.04 (m, 2H), 0.88 (d, J = 6.4 Hz, 3H) | 388.1 |
| | (R)-N-((R)-1-hydroxypropan-2-yl)-4-((7- | | |
| | methoxyquinolin-4- | | |
| | yl)oxy)benzenesulfonimidamide | | |
| 12 | 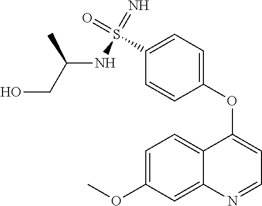 | (400 MHz, DMSO-d6) δ = 8.66 (d, J = 5.3 Hz, 1H), 8.14 (d, J = 9.3 Hz, 1H), 7.99 (d, J = 8.8 Hz, 2H), 7.42 (d, J = 2.4 Hz, 1H), 7.36 (d, J = 8.6 Hz, 2H), 7.30 (dd, J = 2.4, 9.3 Hz, 1H), 6.66 (d, J = 5.1 Hz, 1H), 3.92 (s, 3H), 3.28 − 3.24 (m, 1H), 3.14 − 3.09 (m, 1H), 3.09 − 3.07 (m, 1H), 0.86 (d, J = 6.4 Hz, 3H) | 388.1 |
| | (S)-N-((R)-1-hydroxypropan-2-yl)-4-((7- | | |
| | methoxyquinolin-4- | | |
| | yl)oxy)benzenesulfonimidamide | | |
| 13 | 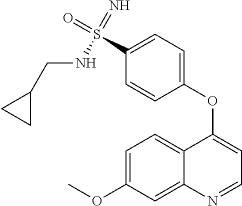 | (400 MHz, METHANOL-d4) δ = 8.62 (d, J = 5.6 Hz, 1H), 8.24 (d, J = 5.2 Hz, 1H), 8.10 (d, J = 6.8 Hz, 2H), 7.42 −7.38 (m, 3H), 7.38 (dd, J = 2.4, 1.2 Hz, 1H), 6.66 (d, J = 5.2 Hz, 1H), 3.99 (s, 3H), 2.81-2.76 (m, 2H), 0.90 − 0.86 (m, 1H), 0.45 − 0.43 (m, 2H), 0.43- 0.12 (m, 2H) | 384.1 |
| | (R)-N-(cyclopropylmethyl)-4-((7- | | |
| | methoxyquinolin-4- | | |
| | yl)oxy)benzenesulfonimidamide | | |
| 14 | 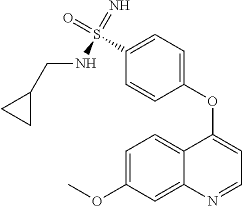 | (400 MHz, METHANOL-d4) δ = 8.61 (d, J = 5.1 Hz, 1H), 8.24 (d, J = 9.3 Hz, 1H), 8.10 (d, J = 8.6 Hz, 2H), 7.40- 7.37 (m, 3H), 7.31 (dd, J = 2.3, 9.2 Hz, 1H), 6.66 (d, J = 5.3 Hz, 1H), 3.98 (s, 3H), 2.81-2.78 (m, 2H), 0.89-0.83 (m, 1H), 0.44-0.42 (m, 2H), 0.13-0.12 (m, 2H) | 384.1 |
| | (S)-N-(cyclopropylmethyl)-4-((7- | | |
| | methoxyquinolin-4- | | |
| | yl)oxy)benzenesulfonimidamide | | |
| 15 | 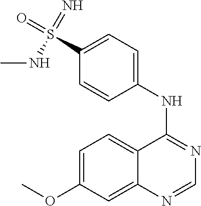 | (400 MHz, DMSO-d6) δ = 8.59 (s, 1H), 8.48 (d, J = 9.0 Hz, 1H), 8.05 (d, J = 8.8 Hz, 2H), 7.83 (d, J = 8.8 Hz, 2H), 7.29 (dd, J = 2.5, 9.2 Hz, 1H), 7.21 (d, J = 2.4 Hz, 1H), 3.92 (s, 3H), 2.36 (s, 3H) | 344.1 |
| | (R)-4-((7-methoxyquinazolin-4- | | |
| | yl)amino)-N- | | |
| | methylbenzenesulfonimidamide | | |
| 16 | 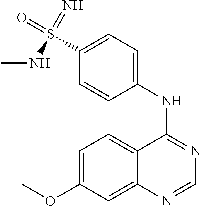 | (400 MHz, DMSO-d6) δ = 8.63 (s, 1H), 8.49 (d, J = 9.3 Hz, 1H), 8.04 (d, J = 8.8 Hz, 2H), 7.85 (d, J = 8.8 Hz, 2H), 7.30 (dd, J = 2.6, 9.3 Hz, 1H), 7.21 (d, J = 2.4 Hz, 1H), 3.92 (s, 3H), 2.83 (s, 3H) | 344.1 |
| | (S)-4-((7-methoxyquinazolin-4- | | |
| | yl)amino)-N- | | |
| | methylbenzenesulfonimidamide | | |
| 17 | 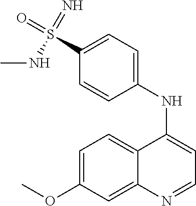 | (400 MHz, DMSO-d6) δ = 8.47 (d, J = 5.1 Hz, 1H), 8.22 (d, J = 9.3 Hz, 1H), 7.82 (d, J = 8.8 Hz, 2H), 7.44 (d, J = 8.8 Hz, 2H), 7.28 (d, J = 2.4 Hz, 1H), 7.21 (dd, J = 2.6, 9.3 Hz, 1H), 7.08 (br d, J = 5.1 Hz, 1H), 3.89 (s, 3H), 2.36 (s, 3H) | 343.1 |
| | (R)-4-((7-methoxyquinolin-4- | | |
| | yl)amino)-N- | | |
| | methylbenzenesulfonimidamide | | |
| 18 | 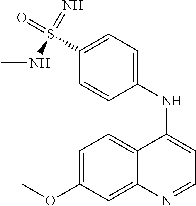 | (400 MHz, DMSO-d6) δ = 8.47 (d, J = 5.3 Hz, 1H), 8.22 (d, J = 9.3 Hz, 1H), 7.80 (d, J = 8.8 Hz, 2H), 7.44 (d, J = 8.6 Hz, 2H), 7.29 (d, J = 2.2 Hz, 1H), 7.20 (dd, J = 2.4, 9.3 Hz, 1H), 7.08 (d, J = 5.3 Hz, 1H), 3.89 (s, 3H), 2.36 (s, 3H) | 343.1 |
| | (S)-4-((7-methoxyquinolin-4- | | |
| | yl)amino)-N- | | |
| | methylbenzenesulfonimidamide | | |
| 19 | 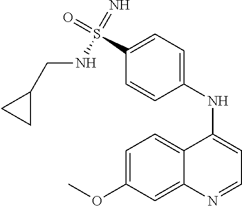 | (DMSO-d6, 400 MHz) δ = 9.18 (s, 1H), 8.49 (d, J = 5.2 Hz, 1H), 8.25 (d, J = 9.2 Hz, 1H), 7.82 (d, J = 8.8 Hz, 2H), 7.42 (d, J = 8.4 Hz, 2H), 7.30 (d, J = 2.4 Hz, 1H), 7.22 (dd, J = 2.8, 9.2 Hz, 1H), 7.06 (d, J = 5.2 Hz, 1H), 6.90 (br s, 1H), 3.91 (s, 3H), 2.65-2.55 (m, 2H), 0.90-0.70 (m, 1H), 0.35-0.30 (m, 2H), 0.15-0.03 (m, 2H) | 381.1 (M − 1) |
| | (R)-N-(cyclopropylmethyl)-4-((7- | | |
| | methoxyquinolin-4- | | |
| | yl)amino)benzenesulfonimidamide | | |
| 20 | 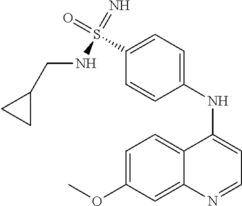 | (DMSO-d6, 400 MHz) δ = 9.18 (s, 1H), 8.49 (d, J = 5.2 Hz, 1H), 8.24 (d, J = 9.2 Hz, 1H), 7.82 (d, J = 8.8 Hz, 2H), 7.42 (d, J = 8.4 Hz, 2H), 7.30 (d, J = 2.4 Hz, 1H), 7.21 (dd, J = 2.8, 9.2 Hz, 1H), 7.06 (d, J = 5.2 Hz, 1H), 6.90 (br s, 1H), 3.99 (br s, 1H), 3.91 (s, 3H), 2.65-2.55 (m, 2H), 0.35- 0.30 (m, 2H), 0.15-0.03 (m, 2H) | 381.1 (M − 1) |
| | (S)-N-(cyclopropylmethyl)-4-((7- | | |
| | methoxyquinolin-4- | | |
| | yl)amino)benzenesulfonimidamide | | |
| 21 | 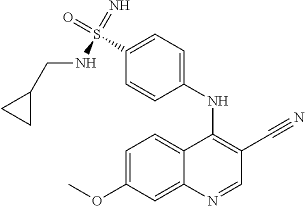 | (CD3OD, 400 MHz) δ = 8.83 (s, 1H), 8.23-8.21 (m, 1H), 8.07-8.05 (m, 2H), 7.45 − 7.42 (m, 3H), 7.39 − 7.36 (m, 1H), 4.02 (s, 1H), 3.04-2.99 (m, 2H), 0.93-0.90 (m, 1H), 0.51-0.47 (m, 2H), 0.22-0.17 (m, 2H), | 408.0 |
| | (S)-4-((3-cyano-7-methoxyquinolin-4- | | |
| | yl)amino)-N- | | |
| | (cyclopropylmethyl)benzenesulfonimidamide | | |
| 22 | 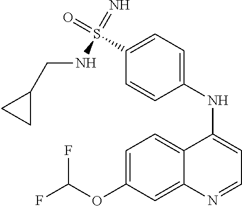 | (DMSO-d6, 400 MHz) δ = 8.49 (d, J = 5.3 Hz, 1H), 8.34 (d, J = 9.3 Hz, 1H), 7.78 (d, J = 8.7 Hz, 2H), 7.55-7.50 (m, 1H), 7.37 − 7.33 (m, 3H), 7.36 (t, 1H), 7.10 (d, J = 5.3 Hz, 1H), 2.55 (d, J = 6.4 Hz, 2H), 0.73 (br s, 1H), 0.27- 0.25 (m, 2H), 0.01-0.00 (m, 2H) | 419.1 |
| | (S)-N-(cyclopropylmethyl)-4-((7- | | |
| | (difluoromethoxy)quinolin-4- | | |
| | yl)amino)benzenesulfonimidamide | | |
| 23 | 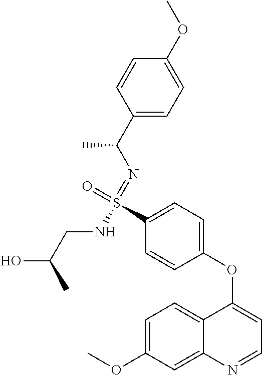 | | 522.4 |
| | (R)-N-((R)-2-hydroxypropyl)-N- | | |
| | ((R)-1-(4-methoxyphenyl)ethyl)-4-((7- | | |
| | methoxyquinolin-4- | | |
| | yl)oxy)benzenesulfonimidamide | | |
| 24 | 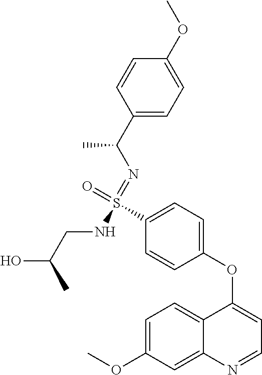 | | 522.4 |
| | (S)-N-((R)-2-hydroxypropyl)-N-((R)-1- | | |
| | (4-methoxyphenyl)ethyl)-4-((7- | | |
| | methoxyquinolin-4- | | |
| | yl)oxy)benzenesulfonimidamide | | |
| 25 | 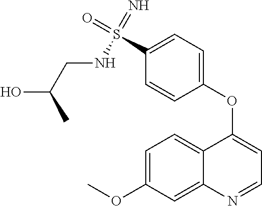 | (400 MHz, DMSO-d6) δ = 8.66 (d, J = 5.1 Hz, 1H), 8.13 (d, J = 9.2 Hz, 1H), 7.96 (d, J = 8.7 Hz, 2H), 7.41 (d, J = 2.2 Hz, 1H), 7.37 (d, J = 8.7 Hz, 2H), 7.30 (dd, J = 2.3, 9.2 Hz, 1H), 6.64 (d, J = 5.1 Hz, 1H), 3.91 (s, 3H), 3.59-3.54 (m, 1H), 2.66 − 2.50 (m, 2H), 1.06-0.98 (d, J = 6.1 Hz, 3H) | 388.1 |
| | (R)-N-((R)-2-hydroxypropyl)-4- | | |
| | ((7-methoxyquinolin-4- | | |
| | yl)oxy)benzenesulfonimidamide | | |
| 26 | 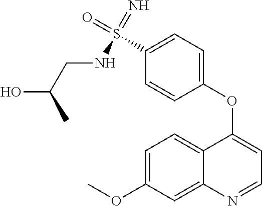 | (400 MHz, DMSO-d6) δ = 8.64 (d, J = 5.1 Hz, 1H), 8.17 (s, 2H), 8.13 (d, J = 9.2 Hz, 1H), 7.95 (d, J = 8.6 Hz, 2H), 7.40 (d, J = 2.1 Hz, 1H), 7.36 (d, J = 8.7 Hz, 2H), 7.30 (dd, J = 2.2, 9.2 Hz, 1H), 6.63 (d, J = 5.1 Hz, 1H), 5.64 (s, 1H), 3.90 (s, 3H), 3.62-3.54 (m, 1H), 2.68-2.60 (m, 2H), 0.98 (d, J = 6.1 Hz, 3H) | 388.1 |
| | (S)-N-((R)-2-hydroxypropyl)-4-((7- | | |
| | methoxyquinolin-4- | | |
| | yl)oxy)benzenesulfonimidamide | | |
| 27 | 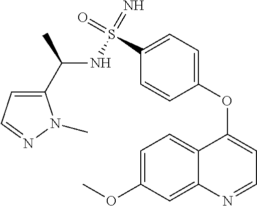 | | 438.1 |
| | (R)-4-((7-methoxyquinolin-4- | | |
| | yl)oxy)-N-((R)-1-(1-methyl-1H- | | |
| | pyrazol-5- | | |
| | yl)ethyl)benzenesulfonimidamide | | |
| 28 | 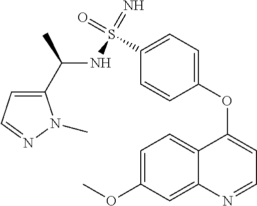 | | 438.1 |
| | (S)-4-((7-methoxyquinolin-4-yl)oxy)-N- | | |
| | ((R)-1-(1-methyl-1 H-pyrazol-5- | | |
| | yl)ethyl)benzenesulfonimidamide | | |
| 29 | 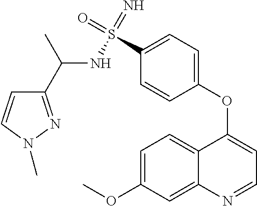 | | 438.1 |
| | (R)-4-((7-methoxyquinolin-4- | | |
| | yl)oxy)-N-(1-(1-methyl-1H- | | |
| | pyrazol-3- | | |
| | yl)ethyl)benzenesulfonimidamide | | |
| 30 | 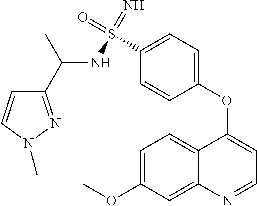 | | 438.1 |
| | (S)-4-((7-methoxyquinolin-4-yl)oxy)-N- | | |
| | (1-(1-methyl-1H-pyrazol-3- | | |
| | yl)ethyl)benzenesulfonimidamide | | |
| 31 | 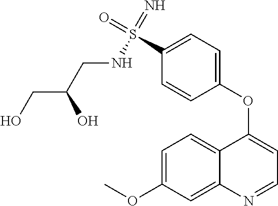 | (CD3OD, 400 MHz) δ = 8.62-8.60 (m, 1H), 8.22-8.20 (d, J = 9.29 Hz, 1H), 8.10-8.08 (m, 2H), 7.44-7.37 (m, 3H), 7.29 (dd, J = 9.17, 2.45 Hz, 1H), 6.67 (d, J = 5.38 Hz, 1H), 3.98 (s, 3H), 3.67-3.53 (m, 1H), 3.53-3.51 (m, 2H), 3.16- 3.02 (m, 1H), 2.95-2.92 (m, 1H) | 404.0 |
| | (R)-N-((R)-2,3-dihydroxypropyl)-4- | | |
| | ((7-methoxyquinolin-4- | | |
| | yl)oxy)benzenesulfonimidamide | | |
| 32 | 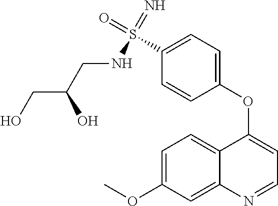 | (CD3OD, 400 MHz) δ = 8.61 (d, J = 5.38 Hz, 1H), 8.22 (d, J = 9.29 Hz, 1H), 8.10-8.07 (m, 2H), 7.39-7.37 (m, 3H), 7.30 (dd, J = 9.17, 2.45 Hz, 1H), 6.67 (d, J = 5.38 Hz, 1H), 3.98 (s, 3H), 3.69-3.66 (m, 1H), 3.52-3.48 (m, 2H), 3.18- 3.03 (m, 1H), 3.07-2.93 (m, 1H | 404.0 |
| | (S)-N-((R)-2,3-dihydroxypropyl)-4- | | |
| | ((7-methoxyquinolin-4- | | |
| | yl)oxy)benzenesulfonimidamide | | |
| 33 | 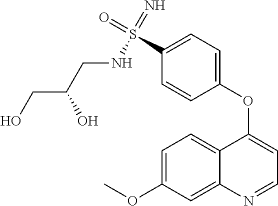 | (CD3OD, 400 MHz) δ = 8.62 (d, J = 5.36 Hz, 1H), 8.24 (d, J = 9.18 Hz, 1H), 8.15-8.03 (m, 2H), 7.47-7.37 (m, 3H), 7.31 (dd, J = 9.24, 2.44 Hz, 1H), 6.69 (d, J = 5.48 Hz, 1H), 4.51 (m, 1H), 3.99 (s, 3H), 3.69-3.66 (m, 1H), 3.52 (dd, J = 5.30, 2.44 Hz, 2H), 3.15- 3.03 (m, 1H), 2.92-2.86 (m, 1H) | 404.0 |
| | (R)-N-((S)-2,3-dihydroxypropyl)-4- | | |
| | ((7-methoxyquinolin-4- | | |
| | yl)oxy)benzenesulfonimidamide | | |
| 34 | 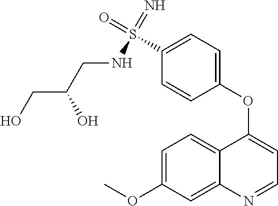 | (CD3OD, 400 MHz) δ = 8.63 (d, J = 5.36 Hz, 1H), 8.25 (d, J = 9.18 Hz, 1H), 8.11-8.09 (m, 2H), 7.42-7.39 (m, 3H), 7.31 (dd, J = 9.24, 2.44 Hz, 1H), 6.70 (d, J = 5.48 Hz, 1H), 3.99 (s, 3H), 3.66-3.54 (m, 1H), 3.53 (dd, J = 5.30, 2.44 Hz, 2H), 3.14-3.04 (m, 1H), 3.00-2.85 (m, 1H), | 404.0 |
| | (S)-N-((S)-2,3-dihydroxypropyl)-4- | | |
| | ((7-methoxyquinolin-4- | | |
| | yl)oxy)benzenesulfonimidamide | | |
| 35 | 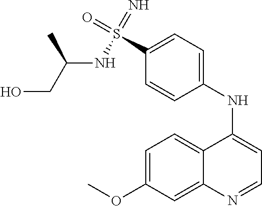 | (400 MHz, METHANOL-d4) δ = 8.43 (d, J = 6.2 Hz, 1H), 8.23 (d, J = 9.0 Hz, 1H), 8.00 (d, J = 8.6 Hz, 2H), 7.51 (d, J = 8.8 Hz, 2H), 7.25 (d, J = 2.8 2H), 7.23 (d, J = 6.2 Hz, 1H), 7.13 (d, J = 5.6 Hz, 1H), 3.99 (s, 3H), 3.48 − 3.46 (m, 2H), 3.36-3.35 (m, 2H), 0.97 (d , 3H) | 387.0 |
| | (R)-N-((R)-1-hydroxypropan-2-yl)-4- | | |
| | ((7-methoxyquinolin-4- | | |
| | yl)amino)benzenesulfonimidamide | | |
| 36 | 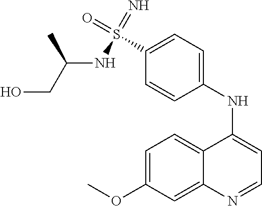 | (400 MHz, METHANOL-d4) δ = 8.41 (d, J = 9.4 Hz, 1H), 8.36 (d, J = 7.0 Hz, 1H), 8.12 (d, J = 8.8 Hz, 2H), 7.62 (d, J = 8.8 Hz, 2H), 7.41 (dd, J = 2.4, 9.4 Hz, 1H), 7.27 (d, J = 2.4 Hz, 1H), 7.02 (d, J = 6.8 Hz, 1H), 4.02 (s, 3H), 3.39 − 3.38 (m, 2H), 3.34 − 3.32 (m, 1H), 1.04 − 1.02 (d, J = 6.4 Hz, 3H) | 387.0 |
| | (S)-N-((R)-1-hydroxypropan-2-yl)-4- | | |
| | ((7-methoxyquinolin-4- | | |
| | yl)amino)benzenesulfonimidamide | | |
| 37 | 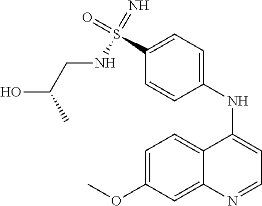 | (400 MHz, DMSO-d6) δ = 14.11 (br s, 1H), 10.79 (s, 1H), 8.61 − 8.54 (m, 2H), 8.05 (d, J = 8.3 Hz, 2H), 7.68 (d, J = 8.6 Hz, 2H), 7.52 (dd, J = 2.4, 9.4 Hz, 1H), 7.37 (s, 1H), 6.95 (d, J = 7.2 Hz, 1H), 3.99 (s, 3H), 3.66-3.58 (m, 1H), 2.79 − 2.72 (m, 2H), 2.07 (s, 1H), 1.02 − 0.98 (d, J = 6.4 Hz, 3H). | 387.1 |
| | (R)-N-((S)-2-hydroxypropyl)-4-((7- | | |
| | methoxyquinolin-4- | | |
| | yl)amino)benzenesulfonimidamide | | |
| 38 | 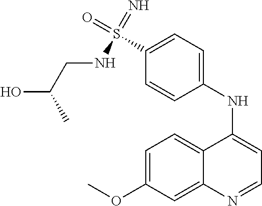 | (400 MHz, DMSO-d6) δ = 14.07(br s, 1H), 10.79 (s, 1H), 8.62 − 8.59 (m, 1H), 8.55 − 8.54 (m, 1H), 8.05 (d, J = 8.6 Hz, 2H), 7.67 (d, J = 8.8 Hz, 2H), 7.52 (dd, J = 2.4, 9.4 Hz, 1H), 7.37 (d, J = 2.4 Hz, 1H), 6.95 (d, J = 7.0 Hz, 1H), 3.99 (s, 3H), 3.66-3.60 (m, 1H), 2.80 − 2.69 (m, 2H), 1.02 − 1.01 (m, 3H) | 387.1 |
| | (S)-N-((S)-2-hydroxypropyl)-4-((7- | | |
| | methoxyquinolin-4- | | |
| | yl)amino)benzenesulfonimidamide | | |
| 39 | 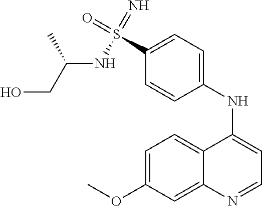 | (400 MHz, DMSO-d6) δ = 8.66 − 8.49 (m, 2H), 8.09 (d, J = 8.8 Hz, 2H), 7.71 (d, J = 8.8 Hz, 2H), 7.50 (dd, J = 2.5, 9.4 Hz, 1H), 7.37 (d, J = 2.4 Hz, 1H), 6.99 (d, J = 7.1 Hz, 1H), 3.99 (s, 3H), 3.37 − 3.23 (m, 2H), 3.23 − 3.12 (m, 1H), 1.00 (d, J = 6.2 Hz, 3H) | 387.1 |
| | (R)-N-((S)-1-hydroxypropan-2-yl)-4- | | |
| | ((7-methoxyquinolin-4- | | |
| | yl)amino)benzenesulfonimidamide | | |
| 40 | 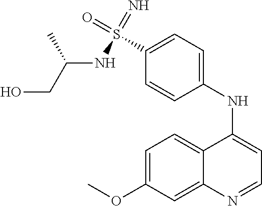 | (400 MHz, DMSO-d6) δ = 8.69 − 8.55 (m, 2H), 8.10 (d, J = 8.6 Hz, 2H), 7.73 (d, J = 8.8 Hz, 2H), 7.51 (dd, J = 2.5, 9.4 Hz, 1H), 7.39 (d, J = 2.4 Hz, 1H), 7.01 (d, J = 7.1 Hz, 1H), 3.98 (s, 3H), 3.41 − 3.32 (m, 2H), 3.27 − 3.18 (m, 1H), 0.96 (d, J = 6.4 Hz, 3H) | 387.1 |
| | (S)-N-((S)-1-hydroxypropan-2-yl)-4- | | |
| | ((7-methoxyquinolin-4- | | |
| | yl)amino)benzenesulfonimidamide | | |
| 41 | 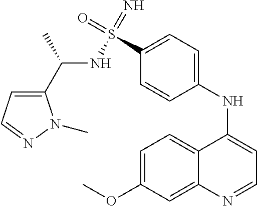 | | 438.1 |
| | (R)-4-((7-methoxyquinolin-4- | | |
| | yl)amino)-N-((S)-1-(1-methyl-1H- | | |
| | pyrazol-5- | | |
| | yl)ethyl)benzenesulfonimidamide | | |
| 42 | 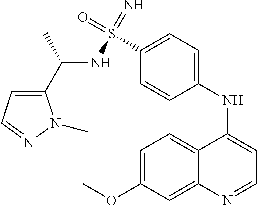 | | 438.1 |
| | (S)-4-((7-methoxyquinolin-4-yl)amino)- | | |
| | N-((S)-1-(1-methyl-1 H-pyrazol-5- | | |
| | yl)ethyl)benzenesulfonimidamide | | |
| 43 | 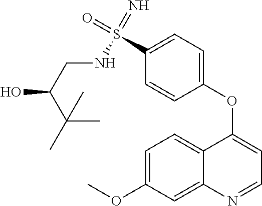 | (400 MHz, DMSO-d6) δ = 8.70 − 8.45 (m, 2H), 8.07 (d, J = 8.6 Hz, 2H), 7.69 (d, J = 8.6 Hz, 2H), 7.50 (dd, J = 2.3, 9.5 Hz, 1H), 7.38 (d, J = 2.3 Hz, 1H), 6.97 (d, J = 7.0 Hz, 1H), 3.99 (s, 3H), 3.46 − 3.01 (m, 2H), 2.67 (d, J = 2.5 Hz, 1H), 0.79 (s, 9H) | 429.1 |
| | (R)-N-((R)-2-hydroxy-3,3- | | |
| | dimethylbutyl)-4-((7- | | |
| | methoxyquinolin-4- | | |
| | yl)oxy)benzenesulfonimidamide | | |
| 44 | 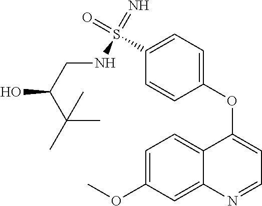 | (400 MHz, DMSO-d6) δ = 8.69 − 8.48 (m, 2H), 8.07 (d, J = 8.8 Hz, 2H), 7.72 (d, J = 8.8 Hz, 2H), 7.49 (dd, J = 2.4, 9.3 Hz, 1H), 7.38 (d, J = 2.2 Hz, 1H), 7.02 (d, J = 6.8 Hz, 1H), 3.98 (s, 3H), 3.20 − 3.05 (m, 2H), 2.79 − 2.62 (m, 1H), 0.79 (s, 9H) | 429.1 |
| | (S)-N-((R)-2-hydroxy-3,3- | | |
| | dimethylbutyl)-4-((7-methoxyquinolin-4- | | |
| | yl)oxy)benzenesulfonimidamide | | |
| 45 | 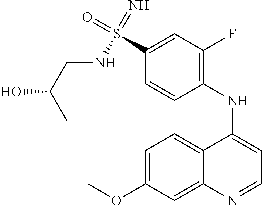 | | 405.1 |
| | (R)-3-fluoro-N-((S)-2-hydroxypropyl)-4-((7- | | |
| | methoxyquinolin-4- | | |
| | yl)amino)benzenesulfonimidamide | | |
| 46 | 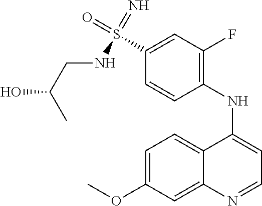 | | 405.1 |
| | (S)-3-fluoro-N-((S)-2-hydroxypropyl)-4-((7- | | |
| | methoxyquinolin-4- | | |
| | yl)amino)benzenesulfonimidamide | | |
| 47 | 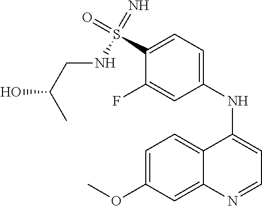 | (400 MHz, DMSO-d6) δ = 8.69 − 8.44 (m, 1H), 8.17 (d, J = 9.4 Hz, 1H), 7.77 (t, J = 7.9 Hz, 1H), 7.31 (s, 1H), 7.27 − 7.11 (m, 4H), 3.90 (s, 3H), 3.80 − 3.55 (m, 1H), 2.81 − 2.68 (m, 2H), 1.00 (d, J = 4.9 Hz, 3H) | 405.1 |
| | (R)-2-fluoro-N-((S)-2-hydroxypropyl)-4-((7- | | |
| | methoxyquinolin-4- | | |
| | yl)amino)benzenesulfonimidamide | | |
| 48 | 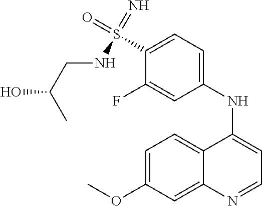 | (400 MHz, DMSO-d6) δ = 8.69 − 8.44 (m, 1H), 8.16 (d, J = 9.3 Hz, 1H), 7.75 (t, J = 8.7 Hz, 1H), 7.31 (d, J = 2.4 Hz, 1H), 7.27 − 7.11 (m, 4H), 3.89 (s, 3H), 3.64 − 3.55 (m, 1H), 2.83 − 2.59 (m, 2H), 0.99 (d, J = 6.2 Hz, 3H) | 405.1 |
| | (S)-2-fluoro-N-((S)-2-hydroxypropyl)-4-((7- | | |
| | methoxyquinolin-4- | | |
| | yl)amino)benzenesulfonimidamide | | |
| 49 | 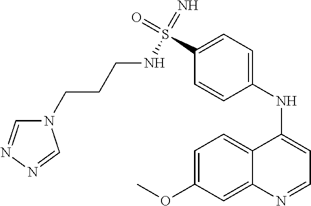 | (400 MHz, METHANOL-d4) δ = 8.50 (s, 2H), 8.41 (d, J = 6.5 Hz, 1H), 8.36 (d, J = 9.4 Hz, 1H), 8.00 − 7.92 (m, 2H), 7.60 − 7.49 (m, 2H), 7.36 (dd, J = 2.5, 9.4 Hz, 1H), 7.29 (d, J = 2.4 Hz, 1H), 7.10 (d, J = 6.5 Hz, 1H), 4.32 − 4.20 (m, 2H), 4.02 (s, 3H), 3.37 − 3.34 (m, 2H) | 424.0 |
| | (R)-N-(3-(4H-1,2,4-triazol-4-yl)propyl)-4-((7- | | |
| | methoxyquinolin-4- | | |
| | yl)amino)benzenesulfonimidamide | | |
| 50 | 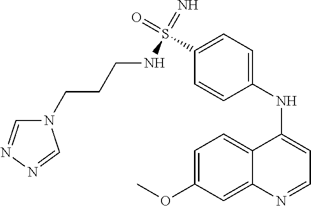 | (400 MHz, METHANOL-d4) δ = 8.51 (s, 2H), 8.45 − 8.38 (m, 2H), 7.98 (d, J = 8.6 Hz, 2H), 7.57 (d, J = 8.6 Hz, 2H), 7.37 (dd, J = 2.3, 9.3 Hz, 1H), 7.31 (d, J = 2.2 Hz, 1H), 7.08 (d, J = 6.7 Hz, 1H), 4.32 − 4.20 (m, 2H), 4.02 (s, 3H), 3.38 − 3.33 (m, 2H) | 424.1 |
| | (S)-N-(3-(4H-1,2,4-triazol-4-yl)propyl)-4-((7- | | |
| | methoxyquinolin-4- | | |
| | yl)amino)benzenesulfonimidamide | | |
| 51 | 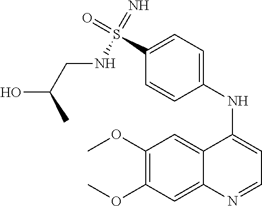 | (400 MHz, METHANOL-d4) δ = 8.43 (s, 1H), 8.32 (d, J = 6.4 Hz, 1H), 8.07 (d, J = 8.6 Hz, 2H), 7.80 (s, 1H), 7.60 (d, J = 8.6 Hz, 2H), 7.30 (s, 1H), 7.11 (d, J = 6.3 Hz, 1H), 4.05 (s, 6H), 3.88 − 3.69 (m, 1H), 2.90 (d, J = 5.9 Hz, 2H), 1.15 (d, J = 6.3 Hz, 3H) | 417.0 |
| | (R)-4-((6,7-dimethoxyquinolin-4- | | |
| | yl)amino)-N-((R)-2- | | |
| | hydroxypropyl)benzenesulfonimidamide | | |
| 52 | 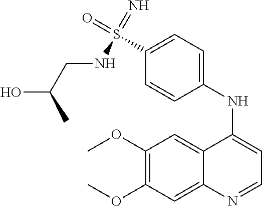 | (400 MHz, METHANOL-d4) δ = 8.44 (s, 1H), 8.32 (d, J = 6.1 Hz, 1H), 8.06 (d, J = 8.6 Hz, 2H), 7.79 (s, 1H), 7.59 (d, J = 8.8 Hz, 2H), 7.30 (s, 1H), 7.11 (d, J = 6.4 Hz, 1H), 4.05 (s, 6H), 3.87 − 3.71 (m, 1H), 2.99 − 2.76 (m, 2H), 1.13 (d, J = 6.3 Hz, 3H) | 417.0 |
| | (S)-4-((6,7-dimethoxyquinolin-4- | | |
| | yl)amino)-N-((R)-2- | | |
| | hydroxypropyl)benzenesulfonimidamide | | |
| 53 | 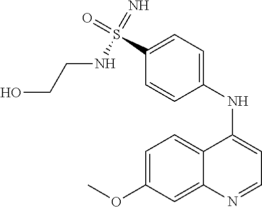 | | 373.2 |
| | (R)-N-(2-hydroxyethyl)-4-((7- | | |
| | methoxyquinolin-4- | | |
| | yl)amino)benzenesulfonimidamide | | |
| 54 | 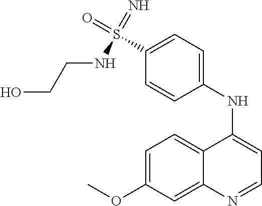 | | 373.2 |
| | (S)-N-(2-hydroxyethyl)-4-((7- | | |
| | methoxyquinolin-4- | | |
| | yl)amino)benzenesulfonimidamide | | |
| 55 | 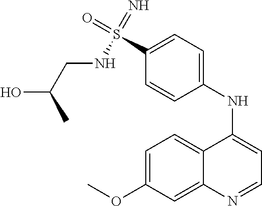 | 1H NMR (400 MHz, DMSO- d6) δ = 8.63 − 8.47 (m, 2H), 8.09 (d, J = 8.7 Hz, 2H), 7.74 (d, J = 8.7 Hz, 2H), 7.48 (dd, J = 2.5, 9.4 Hz, 1H), 7.40 (d, J = 2.5 Hz, 1H), 7.05 (d, J = 6.9 Hz, 1H), 3.98 (s, 3H), 3.68 − 3.64 (m, 1H), 2.87 (d, J = 5.6 Hz, 2H), 1.04 (d, J = 6.3 Hz, 3H) | 387.1 |
| | (R)-N-((R)-2-hydroxypropyl)-4-((7- | | |
| | methoxyquinolin-4- | | |
| | yl)amino)benzenesulfonimidamide | | |
| 56 | 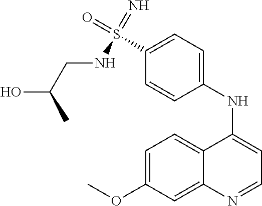 | 1H NMR (400 MHz, DMSO- d6) δ = 8.63 − 8.52 (m, 2H), 8.10 (d, J = 8.6 Hz, 2H), 7.76 (d, J = 8.8 Hz, 2H), 7.47 (dd, J = 2.5, 9.4 Hz, 1H), 7.42 (d, J = 2.4 Hz, 1H), 7.06 (d, J = 7.1 Hz, 1H), 3.97 (s, 3H), 3.72 − 3.62 (m, 1H), 2.86 (d, J = 5.2 Hz, 2H), 1.03 (d, J = 6.4 Hz, 3H) | 387.1 |
| | (S)-N-((R)-2-hydroxypropyl)-4-((7- | | |
| | methoxyquinolin-4- | | |
| | yl)amino)benzenesulfonimidamide | | |
| 57 | 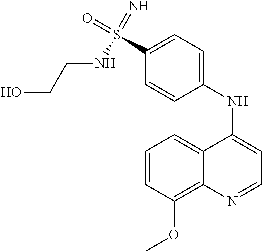 | | 373.2 |
| | (R)-N-(2-hydroxyethyl)-4-((8- | | |
| | methoxyquinolin-4- | | |
| | yl)amino)benzenesulfonimidamide | | |
| 58 | 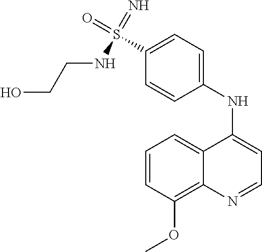 | | 373.2 |
| | (S)-N-(2-hydroxyethyl)-4-((8- | | |
| | methoxyquinolin-4- | | |
| | yl)amino)benzenesulfonimidamide | | |
| 59 | 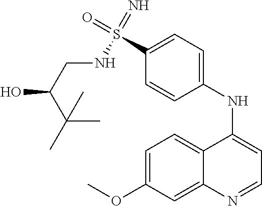 | (400 MHz, DMSO-d6) δ = 8.70 − 8.45 (m, 2H), 8.07 (br d, J = 8.6 Hz, 2H), 7.69 (br d, J = 8.6 Hz, 2H), 7.50 (dd, J = 2.3, 9.5 Hz, 1H), 7.38 (d, J = 2.3 Hz, 1H), 6.97 (d, J = 7.0 Hz, 1H), 3.99 (s, 3H), 3.22 − 3.01 (m, 3H), 2.67 (br d, J = 2.5 Hz, 1H), 0.79 (s, 9H) | 429.1 |
| | (R)-N-((R)-2-hydroxy-3,3- | | |
| | dimethylbutyl)-4-((7-methoxyquinolin-4- | | |
| | yl)amino)benzenesulfonimidamide | | |
| 60 | 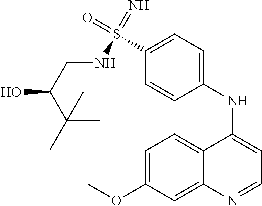 | (400 MHz, DMSO-d6) δ = 8.69 − 8.48 (m, 2H), 8.07 (d, J = 8.8 Hz, 2H), 7.72 (d, J = 8.8 Hz, 2H), 7.49 (dd, J = 2.4, 9.3 Hz, 1H), 7.38 (d, J = 2.2 Hz, 1H), 7.02 (d, J = 6.8 Hz, 1H), 3.98 (s, 3H), 3.20 − 3.05 (m, 2H), 2.79 − 2.62 (m, 1H), 0.79 (s, 9H) | 429.1 |
| | (S)-N-((R)-2-hydroxy-3,3- | | |
| | dimethylbutyl)-4-((7-methoxyquinolin-4- | | |
| | yl)amino)benzenesulfonimidamide | | |
| 61 | 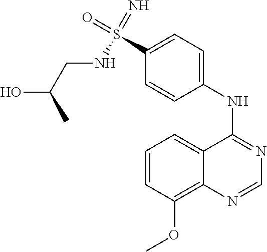 | (400 MHz, DMSO-d6) δ = 8.85 (s, 1H), 8.25 (d, J = 8.1 Hz, 1H), 8.12 − 8.06 (m, 4H), 7.82 (t, J = 8.3 Hz, 1H), 7.68 (d, J = 8.1 Hz, 1H), 4.06 (s, 3H), 3.68 − 3.61 (m, 1H), 2.91 (d, J = 5.9 Hz, 2H), 1.03 (d, J = 6.4 Hz, 3H) | 388.1 |
| | (R)-N-((R)-2-hydroxypropyl)-4-((8- | | |
| | methoxyquinazolin-4- | | |
| | yl)amino)benzenesulfonimidamide | | |
| 62 | 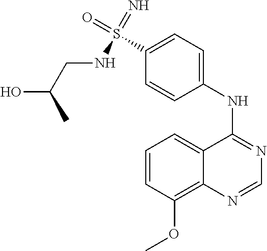 | (400 MHz, DMSO-d6) δ = 8.82 (s, 1H), 8.22 (d, J = 8.4 Hz, 1H), 8.07 − 8.03 (m, 4H), 7.81 (t, J = 8.3 Hz, 1H), 7.67 (d, J = 8.2 Hz, 1H), 4.05 (s, 3H), 3.68 − 3.63 (m, 1H), 2.87 (dd, J = 3.9, 5.8 Hz, 2H), 1.02 (d, J = 6.4 Hz, 3H) | 388.1 |
| | (S)-N-((R)-2-hydroxypropyl)-4-((8- | | |
| | methoxyquinazolin-4- | | |
| | yl)amino)benzenesulfonimidamide | | |
| 63 | 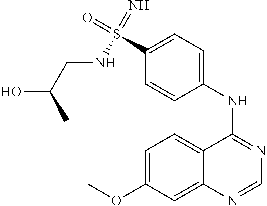 | (400 MHz, DMSO-d6) δ = 10.94 (br s, 1H), 8.88 (s, 1H), 8.63 (d, J = 9.3 Hz, 1H), 8.16-8.06 (m, 2H), 8.06 − 7.99 (m, 2H), 7.48 (dd, J = 2.4, 9.3 Hz, 1H), 7.30 (d, J = 2.4 Hz, 1H), 3.99 (s, 3H), 3.80- 3.60 (m, 1H), 2.92 − 2.81 (m, 2H), 2.07 (s, 1H), 1.07 − 0.98 (m, 3H) | 388.0 |
| | (R)-N-((R)-2-hydroxypropyl)-4-((7- | | |
| | methoxyquinazolin-4- | | |
| | yl)amino)benzenesulfonimidamide | | |
| 64 | 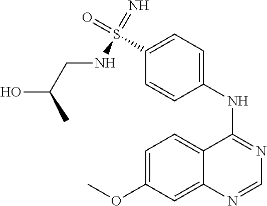 | (400 MHz, DMSO-d6) δ = 10.94 (br s, 1H), 8.88 (s, 1H), 8.63 (d, J = 9.3 Hz, 1H), 8.16-8.06 (m, 2H), 8.06 − 7.99 (m, 2H), 7.48 (dd, J = 2.4, 9.3 Hz, 1H), 7.30 (d, J = 2.4 Hz, 1H), 3.99 (s, 3H), 3.80- 3.60 (m, 1H), 2.92 − 2.81 (m, 2H), 2.07 (s, 1H), 1.07 − 0.98 (m, 3H) | 388.0 |
| | (S)-N-((R)-2-hydroxypropyl)-4-((7- | | |
| | methoxyquinazolin-4- | | |
| | yl)amino)benzenesulfonimidamide | | |
| 65 | 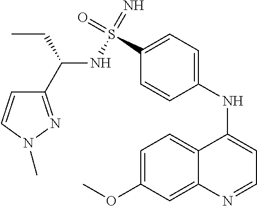 | | 452.1 |
| | (R)-4-((7-methoxyquinolin-4-yl)oxy)-N- | | |
| | ((S)-1-(1-methyl-1H-pyrazol-3- | | |
| | yl)propyl)benzenesulfonimidamide | | |
| 66 | 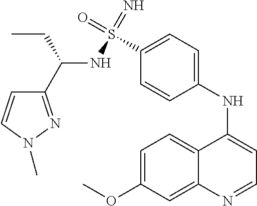 | | 452.0 |
| | (S)-4-((7-methoxyquinolin-4-yl)oxy)-N- | | |
| | ((S)-1-(1-methyl-1 H-pyrazol-3- | | |
| | yl)propyl)benzenesulfonimidamide | | |
| 67 | 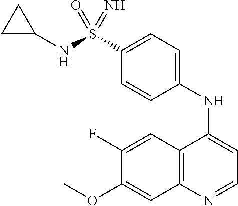 | (400 MHz, DMSO-d6) δ 9.09 (s, 1H), 8.50 (d, J = 5.3 Hz, 1H), 8.17 (d, J = 13.2 Hz, 1H), 7.86 (d, J = 8.7 Hz, 2H), 7.50 (d, J = 8.7 Hz, 1H), 7.44 (d, J = 8.7 Hz, 2H), 7.12 (d, J = 5.3 Hz, 1H), 7.06 (br. s, 1H), 4.12 (submerged br. s, 1H), 4.00 (s, 3H), 2.13 (br. s, 1H), 0.45 − 0.38 (m, 2H), 0.38 − 0.29 (m, 2H) | 387.3 |
| | (S)-N-cyclopropyl-4-((6-fluoro-7-methoxyquinolin-4- | | |
| | yl)amino)benzenesulfonimidamide | | |
| 68 | 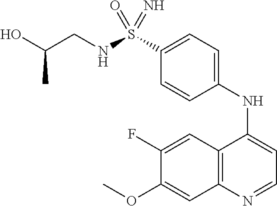 | (400 MHz, DMSO-d6) δ 9.08 (s, 1H), 8.50 (d, J = 5.3 Hz, 1H), 8.16 (d, J = 13.2 Hz, 1H), 7.86 − 7.80 (m, 2H), 7.50 (d, J = 8.7 Hz, 1H), 7.46 − 7.40 (m, 2H), 7.12 (d, J = 5.3 Hz, 1H), 6.69 (br. s, 1H), 4.62 (br. s, 1H), 4.05 (submerged br. s, 1H), 4.00 (s, 3H), 3.65 − 3.54 (m, 1H), 2.74 − 2.54 (m, 2H), 0.99 (d, J = 6.2 Hz, 3H) | 405.3 |
| | (S)-4-((6-fluoro-7-methoxyquinolin-4-yl)amino)-N- | | |
| | ((R)-2-hydroxypropyl)benzenesulfonimidamide | | |
| 69 | 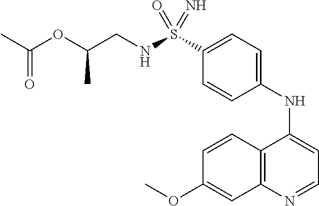 | (400 MHz, DMSO-d6) δ 9.19 (brs, 1H), 8.49 (d, J = 5.3 Hz, 1H), 8.24 (d, J = 9.2 Hz, 1H), 8.20 (s, 1H), 7.82 (d, J = 8.8 Hz, 1H), 7.42 (d, J = 8.8 Hz, 1H), 7.30 (d, J = 2.6 Hz, 1H), 7.21 (dd, J = 9.2, 2.6 Hz, 1H), 7.07 (d, J = 5.2 Hz, 1H), 4.74 (h, J = 6.2 Hz, 1H), 3.91 (s, 3H), 2.87 (d, J = 5.8 Hz, 2H), 1.95 (s, 3H), 1.11 (d, J = 6.3 Hz, 3H) | 429.3 |
| | (R)-1-(((S)-4-((7-methoxyquinolin-4- | | |
| | yl)amino)phenyl)sulfonoamidimidamido) | | |
| | propan-2-yl acetate | | |

### Example 10—ENPP1 Enzymatic Inhibition Assay

The inhibition of ENPP1's phosphodiesterase activity were tested in a purified enzymatic assay using 2′3′-cyclic GAMP as a substrate which allowed for the release of cleaved AMP to be monitored using a luminescent assay. Inhibition of ENPP1 activity by small molecules resulted in a dose-dependent reduction in luminescence.

Putative inhibitors were diluted in assay buffer (50 mM Tris pH 8.0, 250 mM NaCl, 0.5 mM CaCl₂), 1 uM ZnCl₂, 1% DMSO) and pre-incubated for 15 minutes at 37° C. with recombinant protein containing the human ENPP1 enzymatic domain (Viva Biotech). The enzymatic reaction was initiated upon addition of the 2′3′-cGAMP substrate. The final reaction concentrations were 1 nM ENPP1 and 20 uM 2′3′-cGAMP substrate in a 25 uL volume. Inhibitor concentrations ranged from 10 uM to 0.056 nM. The reaction was incubated for 30 minutes at 37° C.

After reacting for 30 minutes, the amount of AMP generated from the cleavage of 2′3′-cGAMP was determined using the Promega AMP-Glo method according to the manufacturer's protocol. First, AMP-Glo reagent 1 was added and incubated with the reaction mix for 1 hour at room temperature. Second, AMP Detection solution was added and the mixture was incubated for an additional hour. The luminescence of the mixture was read on a Perkin Elmer Ensight plate reader.

The maximum and minimum levels of ENPP1 activity were established using no inhibitor and no enzyme controls, respectively. The activity observed using ENPP1 inhibitors was quantified as the percent of activity relative to these controls. IC₅₀ values were calculated using CDD Vault by fitting a sigmoidal variable slope nonlinear regression model to the data.

Table 2 includes IC₅₀ values for ENPP1 inhibition of selected compounds; with compounds having an IC₅₀ of less than 10 nM as A, 10 nM≤B≤100 nM as B, and greater than 100 nM as C.

TABLE 2
EPPN1 IC₅₀ values for selected compounds

| Compound No. | ENPP1 (cGAMP) Inhibition |
|---|---|
| s1 | — |
| 2 | A |
| 3 | B |
| 4 | A |
| 5 | B |
| 6 | A |
| 7 | B |
| 8 | A |
| 9 | B |
| 10 | A |
| 11 | B |
| 12 | A |
| 13 | C |
| 14 | A |
| 15 | B |
| 16 | A |
| 17 | A |
| 18 | A |
| 19 | A |
| 20 | A |
| 21 | B |
| 22 | A |
| 23 | B |
| 24 | A |
| 25 | A |
| 26 | A |
| 27 | B |
| 28 | A |
| 29 | A |
| 30 | A |
| 31 | B |
| 32 | A |
| 33 | B |
| 34 | A |
| 35 | A |
| 36 | A |
| 37 | A |
| 38 | A |
| 39 | A |
| 40 | B |
| 41 | B |
| 42 | A |
| 43 | A |
| 44 | B |
| 45 | B |
| 46 | A |
| 47 | A |
| 48 | A |
| 49 | A |
| 50 | A |
| 51 | B |
| 52 | A |
| 53 | A |
| 54 | A |
| 55 | A |
| 56 | A |
| 57 | C |
| 58 | A |
| 59 | A |
| 60 | A |
| 61 | C |
| 62 | B |
| 63 | B |
| 64 | A |
| 65 | A |
| 66 | A |
| 67 | A |
| 68 | A |
| 69 | A |

### Example 11—Treatment of Mice Bearing MC38 Tumor Xenografts (Study 1)

Ten week old female C57BL/6 mice were implanted subcutaneously in the flank with 5×10⁵ MC38 cells. Tumors were measured every 2-3 days using a caliper. Body weight was measured every 2-3 days. Dosing initiated on Day (when the average tumor volume reached 50 mm³. Five animals were assigned to each treatment group. Animals were dosed orally by gavage with Compound 26 for 10 days and were monitored through Day 32 or until tumors reached 2.000 mm³ in volume.

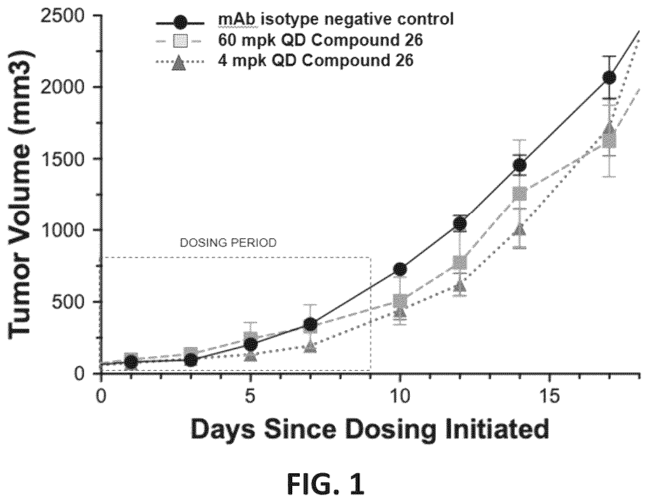

FIG. 1 provides a graph of mean tumor volumes for this set of mice bearing MC38 tumor xenografts. Mice were divided into three groups: (1) control group, who received no treatment (black circles, solid line); (2) mice who received Compound 26 dosed at 4 mg/kg PO QD (grey triangles, dashed lines); and (3) mice who received Compound 26 dosed at 60 mg/kg PO QD (grey squares, dotted line). In each treatment group, compound 26 was administered once a day for 10 consecutive days, at which time, treatment was ceased. FIG. 1 demonstrates that mean tumor volumes were lower for both groups of mice treated with Compound 26 compared to the control group.

### Example 12—Treatment of Mice Bearing MC38 Tumor Xenografts (Study 2)

In this study, 6-8 week old female C57BL/6 mice were implanted subcutaneously in the flank with 5×10⁵ MC38 cells. Tumors were measured every 2-3 days using a caliper. Body weight was measured every 2-3 days. Dosing initiated on Day (when the average tumor volume reached 50 mm³ with 15 animals assigned to each treatment group. Animals were dosed orally by gavage with Compound 26 (4 mg/kg PO QD) or Compound 56 (either 1 mg/kg PO QD or 12 mg/kg PO QD).

A subset of 5 animals from each group were collected on day 10 after the initiation of dosing. Tumors were harvested and lysed via sonication. Supernatants were collected, adjusted for total protein, and assayed using a Luminex cytokine and chemokine panel (Invitrogen) and an IFN-β ELISA. The remaining 10 animals in each group were dosed and monitored through Day 27 or until tumors reached 2 cm in size.

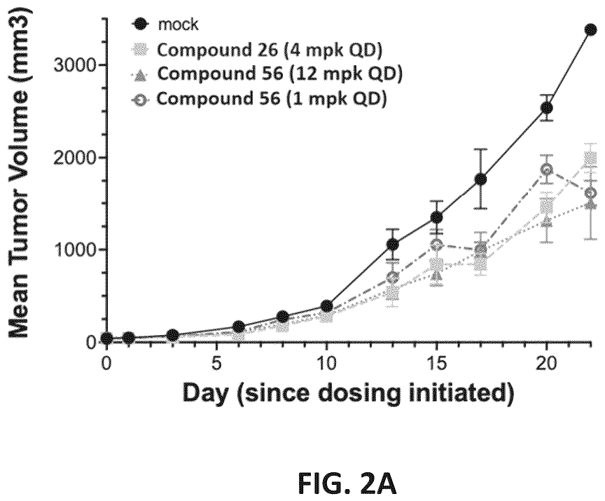

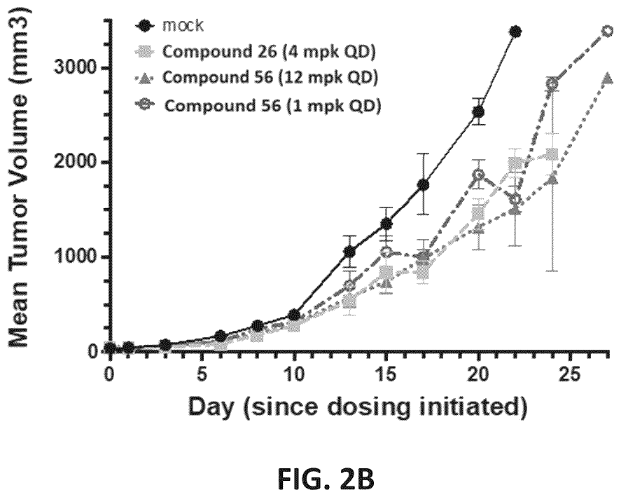

FIG. 2A provides a graph of mean tumor volumes for this set of mice bearing MC38 tumor xenografts. Mice were divided into four groups: (1) control group, who received no treatment (black circles, solid line); (2) mice who received Compound 26 dosed at 4 mg/kg PO QD (grey squares, dashed line); (3) mice who received Compound 56 dosed at 12 mg/kg PO QD (grey triangles, dotted line); and (4) mice who received Compound 56 dosed at 1 mg/kg PO QD (empty circles, dotted dashed line). Mean tumor volumes for all three treated groups were smaller than the mean tumor volume for the untreated group, demonstrating the efficacy of the compounds of the present disclosure. Moreover, the greatest reduction in tumor volume was achieved in mice treated with 12 mg/kg Compound 56, demonstrating a dose-dependent response to treatment. FIG. 2B shows the full data set from FIG. 2A out to 27 days. As seen in FIG. 2B, treatment with compounds of the present disclosure continued to slow tumor growth relative to the control group.

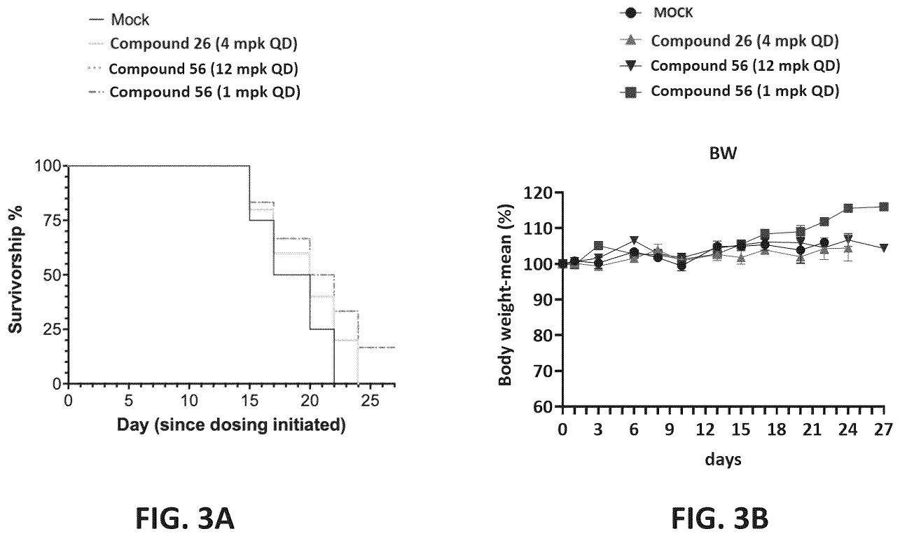

FIG. 3A provides a Kaplan-Meier survival curve for the mice described in FIG. 2, showing survival benefit associated with compounds of the present disclosure. FIG. 3A shows that all untreated mice were deceased by day 22 (solid black lines). In contrast, some mice treated with 4 mg/kg Compound 26 survived out to day 24 (solid grey lines), and at least one mouse from both the 1 mg/kg Compound 56 and 12 mg/kg Compound 56 groups survived beyond day 27 (dotted lines and dashed lines, respectively, overlap on day 27). Furthermore. FIG. 3A demonstrates an increased probability of survival for all treatment groups compared to control from day 15 onward. Thus. FIG. 3A demonstrates that compounds of the present disclosure are effective for treating cancer and increasing survival outcomes.

FIG. 3B provides a graph of body weight for the mice described in FIG. 2. As shown in FIG. 3B, the body weight for all mice (treated and untreated) remained steady throughout the course of the experiment. The results in FIG. 3B therefore demonstrates that that compounds of the present disclosure are well tolerated at therapeutically effective doses.

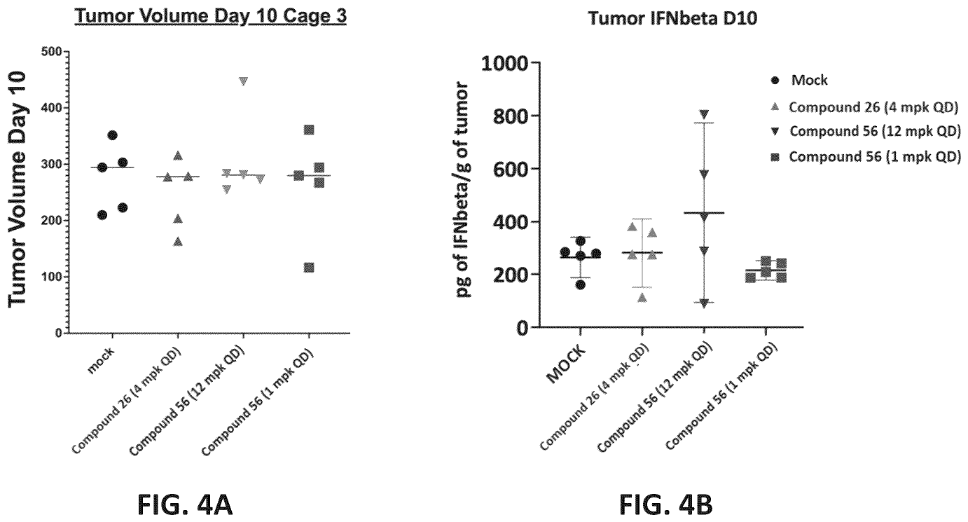

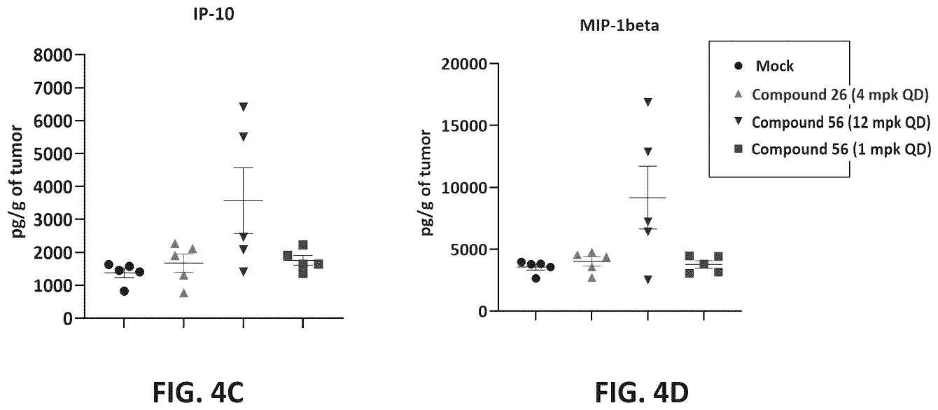

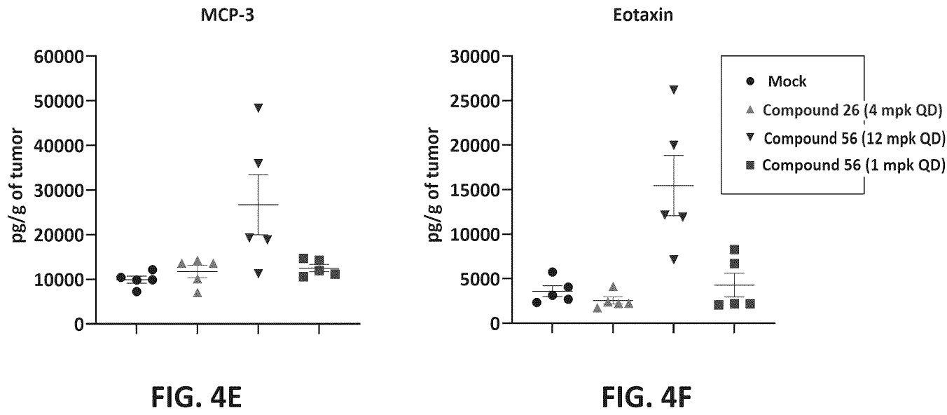

FIG. 4A provides a graph of tumor volume at day 10 for a selected group of mice bearing MC38 tumor xenografts for the following: (1) untreated control group (circles); (2) mice treated with 4 mg/kg QD Compound 26 (triangles); (3) mice treated with 12 mg/kg QD Compound 56 (inverted triangles); and (4) mice treated with 12 mg/kg QD Compound 56 (squares). FIG. 4A shows that the median tumor volume at day 10 was similar across all cohorts. However, closer analysis of the tumor tissue identified marked changes in tumors treatment with compounds of the present disclosure.

FIGS. 4B-F provide graphs demonstrating the intratumoral concentration of each of the following cytokines in tumor tissue derived from mice bearing MC38 tumor xenografts: interferon beta (FIG. 4B), interferon γ-induced protein (FIG. 4C), macrophage inflammatory protein-1-beta (FIG. 4D), monocyte-chemotactic protein 3 (FIG. 4E), and eotaxan (FIG. 4F). Intratumoral concentrations for each of these inflammatory cytokines are provided in picogram of cytokine per gram of tumor tissue. FIGS. 4B-F each provide data for each of the following: (1) untreated control group (circles); (2) mice treated with 4 mg/kg QD Compound 26 (triangles); (3) mice treated with 12 mg/kg QD Compound 56 (inverted triangles); and (4) mice treated with 12 mg/kg QD Compound 56 (squares).

FIG. 4B demonstrates that the median intratumoral concentration of interferon beta (IFN beta) is higher in mice treated with 12 mg/kg (mpk) Compound 56 (>400 pg IFNbeta/g of tumor) compared the control group (“Mock”) and the other treated groups (4 mpk Compound 26 and 1 mpk Compound 56), each of which possessed intratumoral IFNbeta levels of ˜200-300 pg IFNbeta/g of tumor.

FIG. 4C demonstrates that the median intratumoral concentration of interferon γ-induced protein 10 kDa (IP-10) is higher in mice treated with 12 mg/kg (mpk) Compound 56 (3000-4000 pg IP-10/g of tumor) compared the control group (“Mock”) and the other treated groups (4 mpk Compound 26 and 1 mpk Compound 56), each of which possessed intratumoral IP-10 levels of ˜1000-2000 pg IP-10/g of tumor.

FIG. 4D demonstrates that the median intratumoral concentration of macrophage inflammatory protein-1-beta (MIP-1beta) is higher in mice treated with 12 mg/kg (mpk) Compound 56 (˜9000 pg MIP-1 beta/g of tumor) compared the control group (“Mock”) and the other treated groups (4 mpk Compound 26 and 1 mpk Compound 56), each of which possessed intratumoral MIP-1beta levels of <5000 pg MIP-1 beta/g of tumor.

FIG. 4E demonstrates that the median intratumoral concentration of monocyte-chemotactic protein 3 (MCP-3) is higher in mice treated with 12 mg/kg (mpk) Compound 56 (20000-30000 pg MCP-3/g of tumor) compared the control group (“Mock”) and the other treated groups (4 mpk Compound 26 and 1 mpk Compound 56), each of which possessed intratumoral MCP-3 levels of ˜10000-12000 pg MCP-3/g of tumor.

FIG. 4F demonstrates that the median intratumoral concentration of eotaxin is higher in mice treated with 12 mg/kg (mpk) Compound 56 (˜15000 pg eotaxin/g of tumor) compared the control group (“Mock”) and the other treated groups (4 mpk Compound 26 and 1 mpk Compound 56), each of which possessed intratumoral eotaxin levels of <5000 pg MIP-1beta/g of tumor.

The increased cytokine levels observed after treatment with Compound 56 demonstrates that compounds of the present disclosure effectively modulate the immune system in vivo, which is consistent with ENPP1 inhibition.

### Example 13—Treatment of Mice Bearing MC38 Tumor Xenografts (Study 3)

In this study, 6-8 week old female C57BL/6 mice were implanted subcutaneously in the flank with 5×10⁵ MC38 cells. Tumors were measured every 2-3 days using a caliper. Body weight was measured every 2-3 days. Dosing initiated when the average tumor volume reached 50 mm³ with 7 animals assigned to each treatment group. Animals were dosed orally once daily through Day 27 or until tumors reached 2 cm in size.

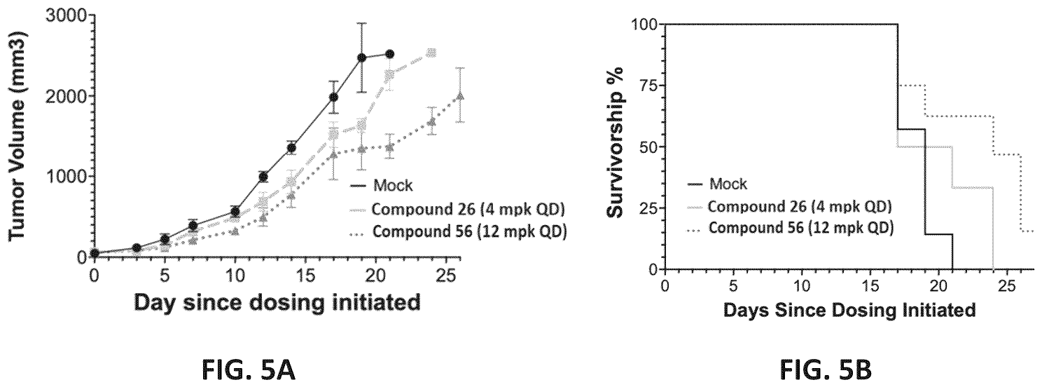

FIGS. 5A-5B provide data for a fourth set of mice bearing MC38 tumor xenografts. FIG. 5A provides a graph of median tumor volumes for this set of mice. Mice were divided into three groups: (1) control group, who received no treatment (circles); (2) mice who received Compound 26 dosed at 4 mg/kg PO QD (squares); and (3) mice who received Compound 56 dosed at 12 mg/kg PO QD (triangles). As shown in FIG. 5A, tumor volumes in mice who received treatment with compounds of the present disclosure was significantly lower than those from the untreated group (compare triangles and squares to circles). For example, at day 20, tumor volumes for the untreated mice were between 2000 and 3000 mm³, while for treated mice had tumor volumes between 1000 and 2000 mm³. FIG. 5A therefore demonstrates that compounds of the present disclosure are effective at significantly slowing tumor growth.

FIG. 5B provides a Kaplan-Meier survival curve for the mice described in FIG. 5A, showing survival benefit associated with compounds of the present disclosure. For example, all untreated mice were deceased by day 21. In contrast, some mice treated with Compound 26 survived out to day 24, while some mice treated with Compound 56 were still alive out through day 27. Thus, FIG. 5B demonstrates that compounds of the present disclosure are effective for treating cancer and increasing survival outcomes.

1. A compound represented by the structure of Formula (I): 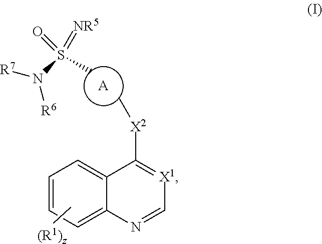
or a pharmaceutically acceptable salt thereof, wherein:
X¹ is selected from N and C(R²);
X² is selected from O, S, C(R³)₂, and N(R⁴);
each R¹ is independently selected from:
halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, —CN; and
C₁₋₆ alkyl, C₂₋₆ alkenyl, and C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle, and 3- to 6-membered heterocycle;
R² is selected from:
hydrogen, halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, —CN; and
C₁₋₆ alkyl, C₂₋₆ alkenyl, and C₂₋₆ alkynyl, each of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —S(O)₂N(R¹²)₂, —N(R¹²)S(O)₂R¹², —S(O)(NR¹²)R¹², —S(NR¹²)₂R¹², —NO₂, ═O, ═S, ═N(R¹²), —CN, C₃₋₆ carbocycle, and 3- to 6-membered heterocycle;
each R³ is independently selected from:
hydrogen, halogen, —OR¹³, —SR¹³, —N(R¹³)₂, —NO₂, and —CN; and
C₁₋₆ alkyl optionally substituted with one or more substituents independently selected from halogen, —OR¹³, —SR¹³, —N(R¹³)₂, —NO₂, and —CN;
R⁴ is selected from hydrogen, C₁₋₆ alkyl, and C₃₋₆ carbocycle wherein the C₁₋₆ alkyl and C₃₋₆ carbocycle are each optionally substituted with one or more substituents independently selected from: halogen, —OR¹⁴, —SR¹⁴, —N(R¹⁴)₂, —NO₂, and —CN;
Ring A is selected from an optionally substituted C₃₋₁₀ carbocycle and an optionally substituted 3- to 10-membered heterocycle wherein substituents on Ring A are independently selected at each occurrence from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, —CN, and C₁₋₆ haloalkyl; and
C₁₋₆ alkyl, C₂₋₆ alkenyl, and C₂₋₆ alkynyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, and —CN;
R⁵, R⁶, and R⁷ are each independently selected from hydrogen, optionally substituted C₁₋₆ alkyl, optionally substituted C₃₋₁₂ carbocycle, and optionally substituted 3- to 12-membered heterocycle, wherein the substituents on R⁵, R⁶, and R⁷ are independently selected at each occurrence from:
halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂ R¹⁶, —S(O)(NR¹⁶)R¹⁶, —S(NR¹⁶)₂R¹⁶, —NO₂, ═O, ═S, ═N(R¹⁶), —CN;
C₁₋₆ alkyl, C₂₋₆ alkenyl, and C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₃₋₁₂ carbocycle, and 3- to 12-membered heterocycle; and
C₃₋₁₂ carbocycle and 3- to 12-membered heterocycle each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)OR¹⁶, —C(O)OR¹⁶, —OC(O)R¹⁶, —S(O)R¹⁶, —S(O)₂R¹⁶, —S(O)₂N(R¹⁶)₂, —N(R¹⁶)S(O)₂R¹⁶, —S(O)(NR¹⁶)R¹⁶, —S(NR¹⁶)₂R¹⁶, —NO₂, ═O, ═S, ═N(R¹⁶), —CN, C₁₋₆ alkyl, C₁₋₆ haloalkyl, C₂₋₆ alkenyl, C₂₋₆ alkynyl, C₃₋₁₂ carbocycle, and 3- to 12-membered heterocycle;
each R¹¹, R¹², R¹³, R¹⁴, R¹⁵, and R¹⁶ is independently selected from:
hydrogen; and
C₁₋₆ alkyl, C₂₋₆ alkenyl, C₂₋₆ alkynyl, C₃₋₈ carbocycle, and 3- to 8-membered heterocycle, each of which is optionally substituted with one or more substituents independently selected from halogen, —OH, —CN, —NO₂, —NH₂, ═O, ═S, —C₁₋₆ haloalkyl, and —O—C₁₋₆ alkyl; and
z is selected from 0-3.

2. The compound or salt of claim 1, wherein z is selected from 1 and 2.

3. The compound or salt of claim 1, wherein X¹ is selected from N, C(H), and C(CN).

4. The compound or salt of claim 1, wherein X² is selected from O and N(H).

5. The compound or salt of claim 1, wherein R¹ is selected from: halogen, —OR¹¹, —N(R¹¹)₂, —C(O)R¹¹, —NO₂, —CN, and C₁₋₆ alkyl optionally substituted with one or more substituents independently selected from halogen, —OR¹¹, —N(R¹¹)₂, —C(O)R¹¹, —NO₂, and —CN.

6. The compound or salt of claim 5, wherein R¹ is selected from —OR¹¹ and R¹¹ is selected from C₁₋₆ alkyl and C₁₋₆ haloalkyl.

7. The compound or salt of claim 6, wherein R¹ is selected from methoxy and —OC(H)F₂.

8. The compound or salt of claim 1, wherein Ring A is selected from C₃₋₆ carbocycle optionally substituted with one or more substituents independently selected from: halogen, —OR¹⁵, —N(R¹⁵)₂, —C(O)R¹⁵, —NO₂, ═O, —CN, and C₁₋₆ haloalkyl.

9. The compound or salt of claim 8, wherein Ring A is phenyl optionally substituted with one or more substituents independently selected from: fluorine, chlorine, CN, and C₁₋₆ haloalkyl.

10. (canceled)

11. The compound or salt of claim 1, wherein two of R⁵, R⁶, and R⁷ are selected from hydrogen and the other of R⁵, R⁶, and R⁷ is selected from C₁₋₆ alkyl optionally substituted with one or more substituents independently selected from:
—OR¹⁶, —N(R¹⁶)₂, —C(O)R¹⁶, —NO₂, ═O, —CN; and
C₃₋₆ carbocycle and 5- to 6-membered heterocycle optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

12. (canceled)

13. The compound or salt of claim 11, wherein R⁷ is C₁₋₆ alkyl optionally substituted with one or more substituents independently selected from: —OR¹⁶, —N(R¹⁶)₂, —C(O)R¹⁶, —NO₂, ═O, and —CN.

14. The compound or salt of claim 13, wherein R⁷ is selected from: methyl,

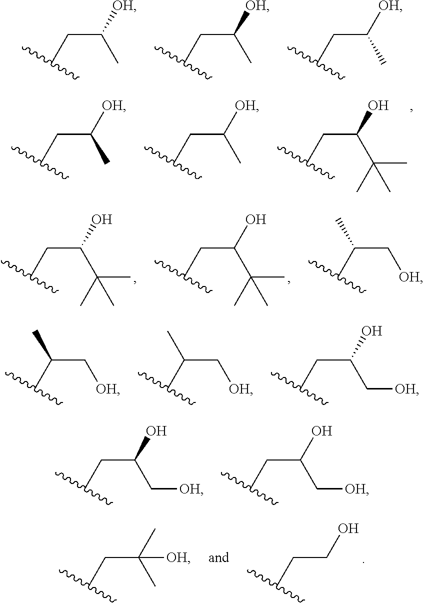

15. The compound or salt of claim 11, wherein R⁷ is C₁₋₆ alkyl optionally substituted with one or more substituents independently selected from saturated C₃₋₆ carbocycle and 5-membered heteroaryl, each of which is optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

16. The compound or salt of claim 15, wherein R⁷ is C₁₋₆ alkyl optionally substituted with one or more substituents independently selected from cyclopropyl, pyrazole and triazole, each of which is optionally substituted with one or more substituents independently selected from —OR¹⁶, —N(R¹⁶)₂, —C(O)R¹⁶, —NO₂, —CN, and C₁₋₆ alkyl optionally substituted with —OR¹⁶, —N(R¹⁶)₂, —C(O)R¹⁶, —NO₂, ═O, and —CN.

17. The compound or salt of claim 16, wherein R⁷ is selected from:

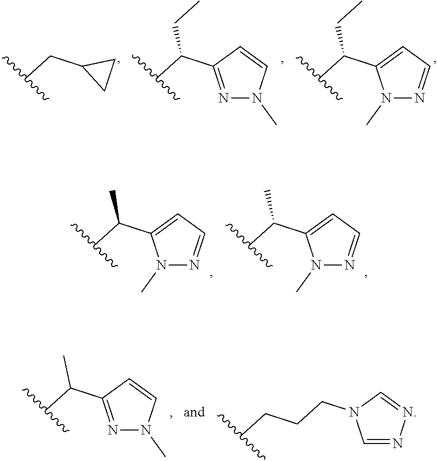

18. The compound or salt of claim 1, wherein the compound of Formula (I) is selected from: 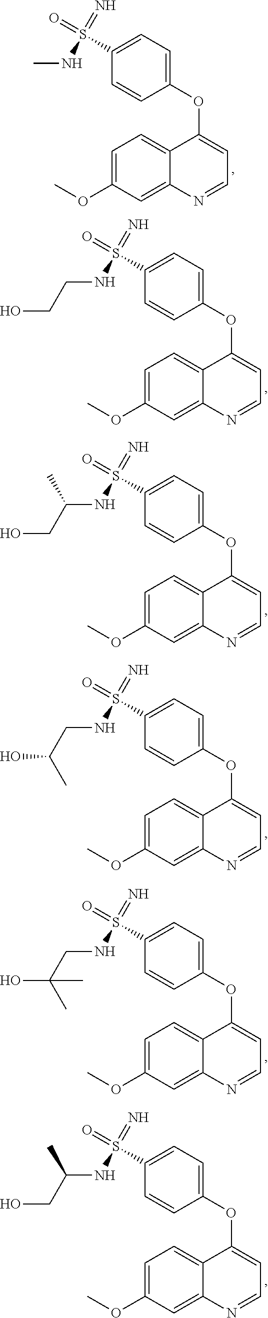 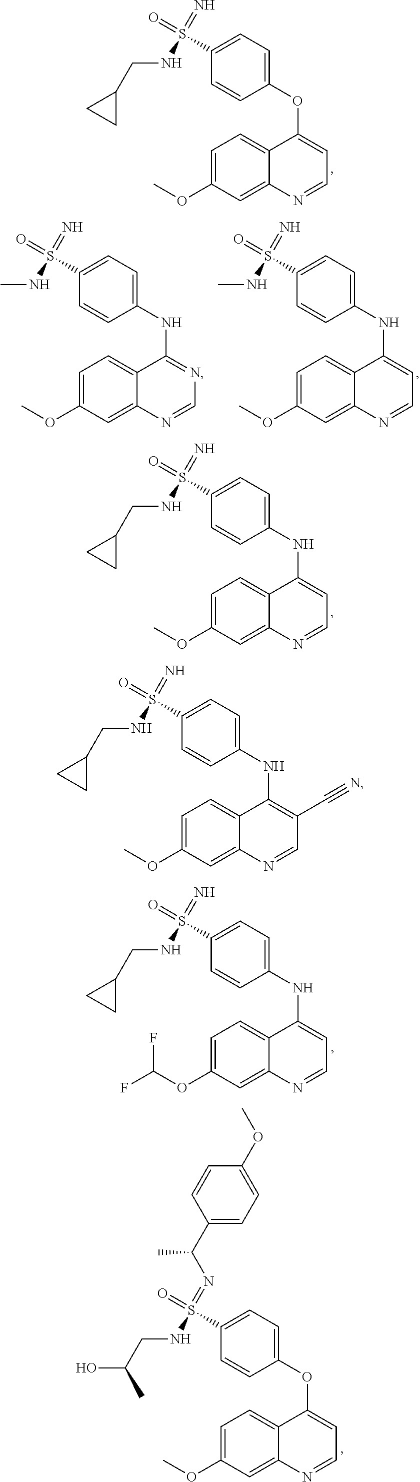 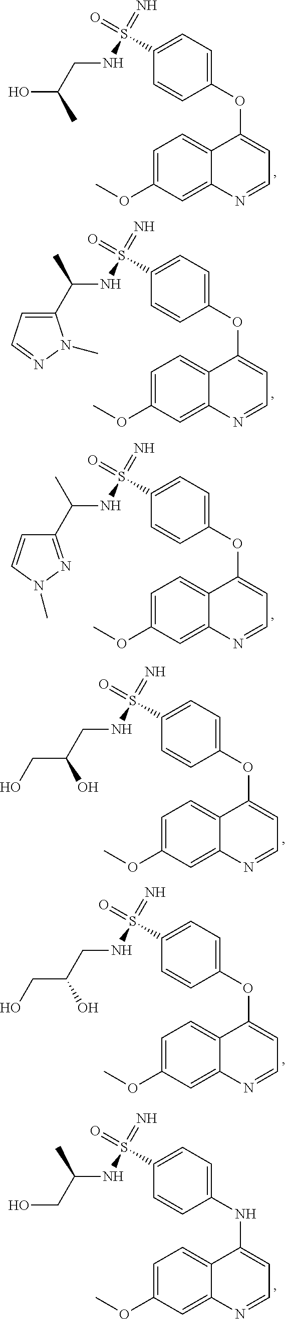 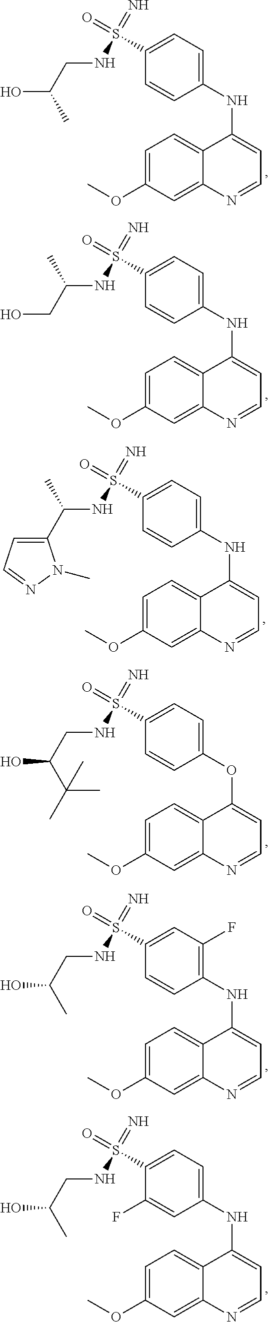 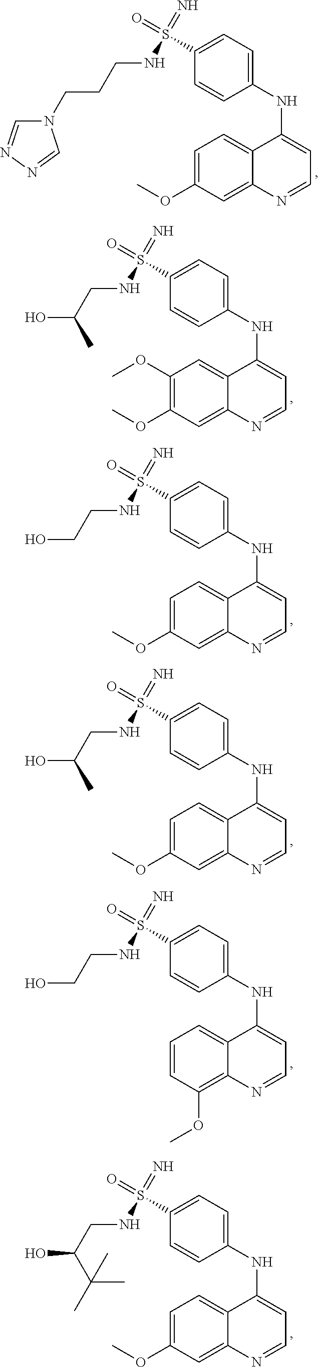 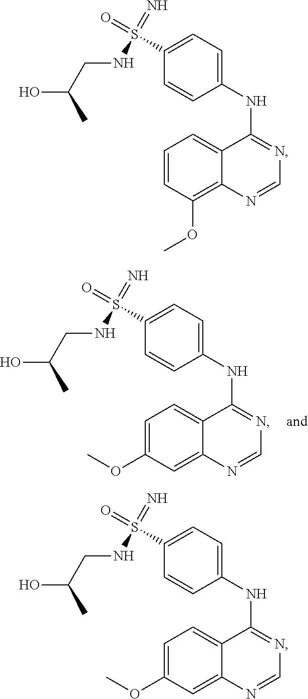
or a pharmaceutically acceptable salt thereof.

19.-38. (canceled)

39. A pharmaceutical composition comprising a compound or salt of claim 1 and a pharmaceutically acceptable excipient.

40. (canceled)

41. A method treating cancer in a subject in need thereof, comprising administrating to the subject a compound or salt of claim 1.

42. The method of claim 41, wherein the cancer comprises a solid tumor.

43. (canceled)

44. The method of claim 41, wherein the cancer is selected from breast cancer, colon cancer, bladder cancer, prostate cancer, ovarian cancer, glioblastoma, and lung cancer.

45.-52. (canceled)
